(19) United States
(12) Patent Application Publication
(10) Pub. No.: US 2025/0277061 A1
(43) Pub. Date: Sep. 4, 2025

(54) MOLECULAR INDEXING OF PROTEINS BY SELF ASSEMBLY (MIPSA) FOR EFFICIENT PROTEOMIC INVESTIGATIONS

(71) Applicant: The Johns Hopkins University, Baltimore, MD (US)

(72) Inventors: Harry B. Larman, Baltimore, MD (US); Joel Credle, Baltimore, MD (US); Jonathan Gunn, Baltimore, MD (US); Puwanat Sankapreecha, Baltimore, MD (US)

(21) Appl. No.: 18/280,166

(22) PCT Filed: Mar. 1, 2022
(86) PCT No.: PCT/US2022/018386

§ 371 Date: Sep. 1, 2023

(51) Int. Cl. C07K 19/00; A61K 38/21; C12N 15/10; C12N 15/62; G01N 33/541; G01N 33/68

(52) U.S. Cl. CPC C07K 19/00; A61K 38/21; C12N 15/10; C12N 15/62; G01N 33/541; G01N 33/68; C07K 2319/735; G01N 2500/02; G01N 2800/52

(57) ABSTRACT

The present disclosure relates to the field of proteomics. More specifically, the present disclosure provides compositions and methods for molecular indexing of proteins by self-assembly. In one aspect, the present disclosure provides a library of self-assembled protein-DNA conjugates. In particular embodiments, each protein-DNA conjugate comprises (a) a cDNA comprising a barcode, wherein the cDNA is conjugated with a ligand that specifically binds a polypeptide tag; and (b) a fusion protein comprising the polypeptide tag and a protein of interest, wherein the ligand is covalently bound to the polypeptide tag.

### CROSS-REFERENCE TO RELATED APPLICATIONS

This application is a 35 U.S.C. § 371 national phase entry of International Patent Application No. PCT/US22/18386 filed Mar. 1, 2022, which claims the benefit of priority under 35 U.S.C. 119 from U.S. provisional application No. 63/155,086, filed Mar. 1, 2021 the disclosures of which are incorporated herein by reference in their entireties.

### GOVERNMENT SUPPORT CLAUSE

This invention was made with government support under grant no. GM127353, awarded by the National Institutes of Health. The government has certain rights in the invention.

### FIELD

The present disclosure relates to the field of proteomics. More specifically, the present disclosure provides compositions and methods for molecular indexing of proteins by self-assembly.

### INCORPORATION-BY-REFERENCE OF MATERIAL SUBMITTED ELECTRONICALLY

The contents of the sequence listing text file named “048317_660001WO_SL.txt”, which was created on Mar. 1, 2022 and is 10,243 bytes in size, are incorporated herein by reference in its entirety.

### BACKGROUND

Unbiased analysis of antibody binding specificities can provide important insights into health and disease states. The present inventors and others have utilized programmable phage display libraries to identify novel autoantibodies, characterize anti-viral immunity and profile allergenic antibodies.(1-4) While phage display has been useful for these and many other applications, most protein-protein, protein-antibody and protein-small molecule interactions require a degree of conformational structure that is not captured using programmable phage display. Profiling conformational protein interactions at proteome scale has traditionally relied on protein microarray technologies. Protein microarrays, however, tend to suffer from high per-assay cost, and a myriad of technical artifacts, including those associated with the high throughput expression and purification of proteins, the spotting of proteins onto a solid support, the drying and rehydration of arrayed proteins, and the slide-scanning fluorescence imaging-based readout.(5, 6) Alternative approaches to protein microarray production and storage have been developed (e.g., Nucleic Acid-Programmable Protein Array, NAPPA(7) or SIMPLEX(8)), but a robust, scalable and cost-effective technology has been lacking.

To overcome the limitations associated with array-based profiling of full-length proteins, the present inventors previously established a methodology called ParalleL Analysis of Translated Open reading frames (PLATO), which utilizes ribosome display of ORFeome libraries.(9) Ribosome display relies on in vitro translation of mRNAs that lack stop codons, stalling ribosomes at the ends of mRNA molecules in a complex with the nascent proteins they encode. PLATO suffers from several key limitations that have limited its utility. An ideal alternative is the covalent conjugation of proteins to short, amplifiable DNA barcodes. Indeed, individually prepared DNA-barcoded antibodies and proteins have been employed in a myriad of applications, as reviewed recently by Liszczak and Muir.(10) One particularly attractive protein-DNA conjugation method involves the HaloTag system, which adapts a bacterial enzyme that forms an irreversible covalent bond with halogen-terminated alkane moieties.(11) Individual DNA-barcoded HaloTag fusion proteins have been shown to greatly enhance sensitivity and dynamic range of autoantibody detection, compared with traditional ELISA.(12) Scaling individual protein barcoding to entire ORFeome libraries would be immensely valuable, but formidable due to high cost and low throughput. Therefore, a self-assembly approach could provide a much more efficient path to library production.

### SUMMARY

The present disclosure is based, at least in part, on the development of a novel molecular display technology, Molecular Indexing of Proteins by Self Assembly (MIPSA), which overcomes key disadvantages of PLATO and other full-length protein array technologies. In particular embodiments, MIPSA produces libraries of soluble full-length proteins, each uniquely identifiable via covalent conjugation to a DNA barcode, flanked by universal PCR primer binding sequences (FIGS. 1A-1C). Barcodes are introduced near the 5′ end of transcribed mRNA sequences, upstream of the ribosome binding site (RBS). Reverse transcription (RT) of the 5′ end of in vitro transcribed mRNA creates a cDNA barcode, which in some embodiments is linked to a haloalkane-labeled RT primer. An N-terminal HaloTag fusion protein is encoded downstream of the RBS, such that in vitro translation results in the intra-complex, covalent coupling of the cDNA barcode to the HaloTag and its downstream open reading frame (ORF) encoded protein product. The resulting library of uniquely indexed full-length proteins can be used for inexpensive proteome-wide interaction studies, such as unbiased autoantibody profiling. As described below, in one embodiment, the present inventors demonstrate the utility of the platform by uncovering known and novel autoantibodies in the plasma of patients with severe COVID-19.

In one aspect, the present disclosure provides methods for efficient proteomic investigation via molecular indexing of proteins by self-assembly (MIPSA). In one embodiment, a method comprises the steps of (a) transcribing a vector library into messenger ribonucleic acid (mRNA), wherein the vector library encodes a plurality of proteins, and wherein each vector of the vector library comprises in the 5′ to 3′ direction: (i) a polymerase transcriptional start site; (ii) a barcode; (iii) a reverse transcription primer binding site; (iv) a ribosome binding site (RBS); and (v) a nucleotide sequence encoding a fusion protein comprising (1) a polypeptide tag and (2) a protein, wherein the polypeptide tag specifically binds a ligand; (b) reverse transcribing the 5′ end of the mRNA using a primer that binds upstream of the RBS, wherein the primer is conjugated with the ligand that specifically binds the polypeptide tag of the fusion protein, and wherein a complementary deoxyribonucleic acid (cDNA) is formed comprising the ligand, primer and barcode; and (c) translating the mRNA, wherein the ligand of the cDNA binds the polypeptide tag of the fusion protein. In a specific embodiment, the vector library is nicked prior to step (a). In another specific embodiment, the vector further comprises (vi) an endonuclease site for vector linearization and the vector library is linearized prior to step (a).

In another aspect, the present disclosure provides a self-assembled protein-DNA conjugate composition. In specific embodiments, the present disclosure provides a library of self-assembled protein-DNA conjugates. In particular embodiments, each protein-DNA conjugate comprises (a) a cDNA comprising a barcode, wherein the cDNA is conjugated with a ligand that specifically binds a polypeptide tag; and (b) a fusion protein comprising the polypeptide tag and a protein of interest, wherein the ligand is covalently bound to the polypeptide tag.

In certain embodiments, the polypeptide tag comprises haloalkane dehalogenase or O⁶-alkylguanine-DNA-alkyltransferase. In a specific embodiment, the polypeptide tag comprises a HALO-tag and the ligand comprises a HALO-ligand. In a more specific embodiment, the HALO-tag comprises the amino acid sequence set forth in SEQ ID NO: 22. In other embodiments, the HALO-ligand comprises one of:

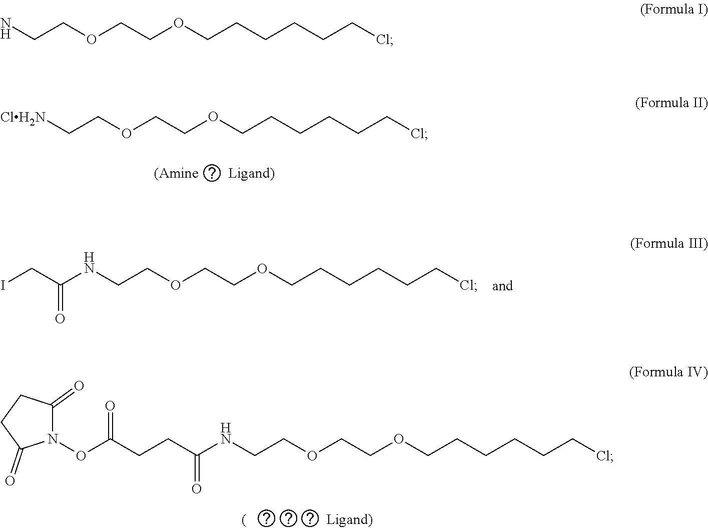

In an alternative embodiment, the polypeptide tag comprises a SNAP-tag and the ligand comprises a SNAP-ligand. In a more specific embodiment, the SNAP-tag comprises the amino acid sequence set forth in SEQ ID NO:23. In other embodiments, the SNAP-ligand comprises benzylguanine or a derivative thereof.

In a further embodiment, the polypeptide tag comprises a CLIP-tag and the ligand comprises a CLIP-ligand. In a more specific embodiment, the CLIP-tag comprises the amino acid sequence set forth in SEQ ID NO:24. In other embodiments, the CLIP-ligand comprises benzylcytosine or a derivative thereof.

The present disclosure also provides methods for using the library of self-assembled protein-DNA conjugates. In one embodiment, a method for studying protein-protein interactions comprises the step of performing a pull-down assay of the library of protein-DNA conjugates with a protein of interest. In another embodiment, a method for studying protein-small molecule interactions comprises the step of performing a pull-down assay of the library of protein-DNA conjugates with a small molecule. In yet another embodiment, a method comprises the step of performing an immunoprecipitation of the library of protein-DNA conjugates with antibodies obtained from a biological sample. In a further embodiment, a method for identifying the target of a first small molecule comprises the steps of (a) incubating the library of protein-DNA conjugates with the first small molecule that binds its target(s) and (b) performing a pull-down assay of the library of step (a) with a second small molecule, wherein the first small molecule bound to its target(s) blocks the binding of the second small molecule. In a more specific embodiment, more than one small molecule is used in the pull-down assay of step (b).

In yet another aspect, the present disclosure provides vectors and self-assembled protein display libraries comprising of plurality of vectors, wherein each vector comprises a nucleic acid sequence that encodes a protein of interest. In one embodiment, a vector comprises along the 5′ to 3′ direction (a) a polymerase transcriptional start site; (b) a barcode; (c) a reverse transcription primer binding site; (d) a RBS; and (e) a nucleotide sequence encoding a fusion protein comprising (i) a polypeptide tag and (ii) a protein of interest, wherein the polypeptide tag specifically binds a ligand.

In particular embodiments, the vector further comprises an endonuclease site for vector linearization. In other embodiments, the vector further comprises (vii) a stop codon.

In a specific embodiment, the barcode is flanked by binding sites for polymerase chain reaction (PCR) primers. In an alternative embodiment, the barcode comprises binding sites for PCR primers.

In another embodiment, the RBS comprises an internal ribosome entry site. In particular embodiments, the polypeptide tag is fused to the N-terminal end of the protein of interest. In other embodiments, the polypeptide tag is fused to the C-terminal end of the protein of interest.

The present disclosure also provides methods for using the self-assembled protein display libraries. In certain embodiments, a method comprises the steps of (a) transcribing a linearized or nicked plurality of vectors comprising a self-assembled protein display library to produce mRNA; (b) reverse transcribing the 5′ end of the mRNA to produce cDNA comprising the barcodes using a primer conjugated to the ligand; and (c) translating the mRNA, wherein the polypeptide tag of the fusion protein covalently binds the ligand conjugated to the cDNA comprising the barcode.

In another aspect, the present disclosure provides methods for treating COVID-19. In one embodiment, a method for treating a patient having severe COVID-19 comprises the step of administering to the patient an effective amount of interferon therapy, wherein autoantibodies that neutralize IFN-λ3 are detected in a biological sample obtained from the patient. In another embodiment, a method for treating a patient having severe COVID-19 comprises the steps of (a) detecting autoantibodies that neutralize IFN-λ3 in a biological sample obtained from the patient; and (b) treating the patient with an effective amount of interferon therapy. In a further embodiment, a method for identifying a COVID-19 patient who would benefit from interferon therapy comprises the step of detecting autoantibodies that neutralize IFN-λ3 in a biological sample obtained from the patient. In particular embodiments, the interferon therapy comprises interferon lambda (IFN-λ) or interferon beta (IFN-β). In specific embodiments, interferon lambda (IFN-λ) or interferon beta (IFN-β) is pegylated.

### BRIEF DESCRIPTION OF THE DRAWINGS

FIGS. 1A-1G demonstrate the MIPSA method. FIG. 1A: Schematic of the recombined pDEST-MIPSA vector with key components highlighted: unique clonal identifier (UCI, blue), ribosome binding site (RBS, yellow), N-terminal HaloTag (purple), FLAG epitope (orange), open reading frame (ORF, green), and the I-SceI restriction endonuclease site (black) for vector linearization. FIG. 1B: Schematic showing in vitro transcribed (IVT) RNA from the vector template shown in (FIG. 1A). Isothermal base-balanced UCI sequence: (SW)₁₈-AGGGA-(SW)₁₈. FIG. 1C: Cell-free translation of the RNA-cDNA shown in (FIG. 1B). HaloTag protein forms a covalent bond with the HaloLigand-conjugated UCI-containing cDNA in cis during translation. FIG. 1D: RT primer positions tested for impact on translation. FIG. 1E: α-FLAG western blot analysis of translation in presence of RT primers depicted in (FIG. 1D) (NC, negative control, no RT primer). FIG. 1F: Western blot analysis of TRIM21 protein translated from RNA carrying the UCI-cDNA primed from the −32 position, either conjugated (+) or not (−) with the HaloLigand. Sjögren's Syndrome, SS; Healthy Control, HC. FIG. 1G: qPCR analysis of the IPed TRIM21 UCI. Fold-difference is by comparison with the HaloLigand (−) HC IP.

FIGS. 2A-2D demonstrate the Cis-versus trans-UCI conjugation. FIG. 2A: IVT-RNA encoding TRIM21 or GAPDH with their distinct UCI barcodes were translated before or after mixing at a 1:1 ratio. qPCR analysis of the IPs using UCI-specific primers, reported as fold-change versus IP with HC plasma, when the IVT-RNA was mixed post-translation. FIG. 2B: IVT-RNA encoding TRIM21 (black UCI) and GAPDH (gray UCI) were mixed 1:1 into a background of 100-fold excess GAPDH (white UCI) and then translated as a mock library. Sequencing analysis of the IPs, reported as fold-change versus the HC IP of the 100×GAPDH. FIG. 2C: hORFeome MIPSA library containing spiked-in TRIM21, IPed with SS plasma and compared to average of 8 mock IPs (no plasma input). The TRIM21 UCI is shown in red. FIG. 2D: Relative fold difference of TRIM21 UCI in SS versus HC IPs, determined by sequencing.

FIGS. 3A-3D demonstrate the construction of the UCI-ORF dictionary. FIG. 3A: (i) Tagmentation randomly inserts adapters into the MIPSA vector library. (ii) Utilizing a PCR1 forward primer and the reverse primer of the tagmentation-inserted adapter, DNA fragments are amplified and size selected to be ˜1.5 kb, which captures the 5′ terminus of the ORF. (iii) These fragments are amplified with a P5-containing PCR2 forward primer and a P7 reverse primer. (iv) Illumina sequencing is used to read the UCI and the ORF from the same fragment, thus enabling their association in the dictionary. FIG. 3B: The number of monospecific UCIs is shown for each member of pDEST-MIPSA hORFeome library, superimposed on the length of the ORFs. FIG. 3C: Histogram of ORF representations in the library according to their aggregated UCI-associated read counts. Vertical red lines show +/−10× the median UCI-associated read count. FIG. 3D: IP of hORFeome MIPSA library using Sjögren's Syndrome (SS) plasma is compared to the average of 8 mock IPs. Sequencing read count for each UCI are plotted. UCIs associated with the two GAPDH isoforms (filled black) and spiked-in TRIM21 (red) are indicated.

FIGS. 4A-4C demonstrate the MIPSA analysis of autoantibodies in severe COVID-19. FIG. 4A: Boxplots showing total numbers of autoreactive proteins in plasma from healthy controls, mild-moderate COVID-19 patients, or severe COVID-19 patients. * indicates p<0.05 from a one-tailed t-test to compare means. FIG. 4B: Hierarchal cluster map of all proteins represented by at least 2 reactive UCIs in at least 1 severe COVID-19 plasma, but not more than 1 control (healthy or mild-moderate COVID-19 plasma). FIG. 4C: MIPSA analysis of autoantibodies in 10 inclusion body myositis (IBM) patients and 10 healthy controls (HCs), using the hORFeome library. Fold change of IPed 5′-nucleotidase, cytosolic 1A (NT5C1A), measured both as UCI-qPCR fold change (relative to average of 10 HCs) and as sequencing fold change (relative to mock IPs).

FIGS. 5A-5H demonstrate that MIPSA detects known and novel neutralizing interferon autoantibodies. FIGS. 5A-5C: Scatterplots highlighting reactive interferon UCIs for three severe COVID-19 patients. FIG. 5D: Summary of interferon reactivity detected in 5 of 55 individuals with severe COVID-19. Hits fold-change values (color of cell) and the number of reactive UCIs (number in cell) are provided. FIGS. 5E-5F: Recombinant interferon alpha 2 (IFN-α2) or interferon lambda 3 (IFN-λ3) neutralizing activity of the same patients shown in FIG. 5D. Plasma were pre-incubated with 100 U/ml of IFN-α2 or 1 ng/ml of IFN-λ3 prior to incubation with A549 cells. Fold changes of the interferon stimulated gene, MX1, were calculated by RT-qPCR relative to unstimulated cells. GAPDH was used as a housekeeping control gene for normalization. Red bars indicate which samples are predicted by MIPSA to have neutralizing activity for each interferon. FIG. 5G: PhIP-Seq analysis of interferon autoantibodies in the 5 patients of FIG. 5D (row and column orders maintained). Hits fold-change values (color of cell) and the number of reactive peptides (number in cell) are provided. FIG. 5H: Epitopefindr analysis of the PhIP-Seq reactive type I interferon 90-aa peptides.

FIGS. 6A-6C demonstrate the HaloLigand conjugation to the reverse transcription primer. FIG. 6A: On the top is the oligonucleotide reverse transcription (RT) primer sequence modified with a 5′ primary amine. Below is the HaloLigand with a reactive succinimidyl ester group, separated by one ethylene glycol moiety (02). The succinimidyl ester reacts with the primary amine to form an amide-bond between the RT primer and the HaloLigand, resulting in the HaloLigand-conjugated RT primer. FIG. 6B: HPLC chromatogram of the RT primer without the HaloLigand modification. FIG. 6C: HPLC chromatogram of the RT primer with the HaloLigand modification after purification. The conjugated product elutes later due to increased hydrophobicity conferred by the modification.

FIGS. 7A-7C demonstrate the cis versus trans UCI-ORF associations. Schematic of cis FIG. 7A, versus trans FIG. 7B, UCI-ORF conjugation during translation of a MIPSA IVT-RNA library. FIG. 7C: Left panel: 50% cis conjugates (“C”) composed of the correct protein-UCI associations (e.g. blue UCI with blue protein). Middle panel: unconjugated proteins then randomly associates with unconjugated UCIs in trans (“T”). Right panel: the ratio of correctly to incorrectly IPed UCIs in this two-species experiment is 3:1 (75%: 25%), similar to experimental observations (FIG. 2A).

FIG. 8 shows the two-plex translation and IP of TRIM21 and GAPDH. TRIM21 (T) and GAPDH (G) IVT-RNA-cDNA were translated either separately or together and then subjected to IP with healthy control (HC) or Sjogren's Syndrome (SS) plasma. Analysis was by immunoblotting with the M2 antibody that recognizes the common FLAG epitope tag that links the HaloTag to the protein.

FIG. 9 demonstrates the sequence homology of interferons. Pairwise blastp alignment bitscore matrix for all interferon (IFN) proteins shown in FIG. 5D.

FIGS. 10A-10C demonstrate the reproducibility and linearity of MIPSA detection of patient P2's autoantibodies. FIG. 10A: Mean and standard deviation of the 100 ORF fold changes for all consistently reactive monospecific UCIs (fold change >3 in all 3 replicates). The values to the right of the error bars are the coefficients of variation. FIG. 10B: Numbers of overlapping reactive monospecific UCIs over three independent MIPSA analyses of P2 plasma. Areas are proportional to numbers of hits. FIG. 10C: Mean ORF fold changes for P2 plasma, compared to P2 plasma diluted 10-fold into a background of a healthy control plasma. Dot sizes depict the numbers of reactive UCIs corresponding to each ORF.

FIGS. 11A-11B demonstrate the titration-based estimate of patient P2's interferon autoantibody levels. Mouse monoclonal blocking antibodies were used at different concentrations in the cell-based IFN neutralization assay: FIG. 11A: IFN-α2 and FIG. 11B IFN-λ3. Neutralization curves were fit and used to estimate patient P2's corresponding interferon autoantibody levels. The plasma dilutions shown were selected to be within the dynamic range of the assay; neutralizing activity of P2 plasma at the dilution shown was assayed in triplicate.

FIGS. 12A-12C demonstrate the MIPSA analysis of interferon antibodies in serial dilution. Summary of interferon reactivity detected by MIPSA in serially diluted P2 plasma (FIG. 12A), IFN-α2 mAb (FIG. 12B), and IFN-λ3 mAb (FIG. 12C). Hits fold-change values (color of cell) and the number of reactive UCIs (number in cell) are provided as in FIG. 5D.

FIG. 13 demonstrates that IFN-λ3 autoantibodies do not efficiently neutralize IFN-λ1. The IFN-λ3 neutralizing activity of patient P2's plasma was compared to its IFN-λ1 neutralizing activity. Neutralization of IFN-λ3 was complete and partial at 1:10 and 1:100 dilutions, respectively. Neutralization of IFN-λ1 was partial and not detected (ND) at 1:10 and 1:100 dilutions, respectively.

### DETAILED DESCRIPTION

It is understood that the present disclosure is not limited to the particular methods and components, etc., described herein, as these may vary. It is also to be understood that the terminology used herein is used for the purpose of describing particular embodiments only, and is not intended to limit the scope of the present disclosure. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this disclosure belongs. Specific methods, devices, and materials are described, although any methods and materials similar or equivalent to those described herein can be used in the practice or testing of the present disclosure.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present disclosure.

The present inventors herein describe a novel molecular display technology for full length proteins, which provides key advantages over protein microarrays, PLATO, and alternative techniques. In particular embodiments, MIPSA utilizes self-assembly to produce a library of proteins, linked to relatively short (e.g., 158 nt) single stranded DNA barcodes via, for example, the 25 kDa HaloTag domain. This compact barcoding approach is likely to find numerous applications not accessible to alternative display formats with bulky linkage cargos (e.g., yeast, phage, ribosomes, mRNAs). Indeed, individually conjugating minimal DNA barcodes to proteins, especially antibodies and antigens, has already proven useful in several contexts, including CITE-Seq,(25) LIBRA-seq,(26) and related methodologies.(22, 27) At proteome scale, MIPSA enables unbiased analyses of protein-antibody, protein-protein, and protein-small molecule interactions, as well as studies of post-translational modification, such as hapten modification studies or protease activity profiling, for example. Key advantages of MIPSA include its high throughput, low cost, simple sequencing library preparation, and stability of the protein-DNA complexes (important for both manipulation and storage of display libraries). Importantly, MIPSA can be immediately adopted by low-complexity laboratories, since it does not require specialized training or instrumentation, simply access to a high throughput DNA sequencing instrument or facility.

Complementarity of MIPSA and PhIP-Seq. Display technologies frequently complement one another, but may not be amenable to routine use in concert. MIPSA is more likely than PhIP-Seq to detect antibodies directed at conformational epitopes on proteins expressed well in vitro. This was exemplified by the robust detection of interferon alpha autoantibodies via MIPSA, described below, which were not detected via PhIP-Seq. PhIP-Seq, on the other hand, is more likely to detect antibodies directed at less conformational epitopes contained within proteins that are either absent from an ORFeome library or cannot be expressed well in bacterial lysate. Because MIPSA and PhIP-Seq naturally complement one another in these ways, the present inventors designed the MIPSA UCI amplification primers to be the same as those the present inventors have used for PhIP-Seq. Since the UCI-protein complex is stable-even in bacterial phage lysate-MIPSA and PhIP-Seq can readily be performed together in a single reaction, using a single set of amplification and sequencing primers. The natural compatibility of these two display modalities will therefore lower the barrier to leveraging their synergy.

Variations of the MIPSA system. A key aspect of MIPSA involves the bonding of a protein to its associated UCI in cis, compared to another library member's UCI in trans. Here, the present inventors have utilized covalent bonding via the HaloTag/HaloLigand system, but there are others that could work as well. For instance, the SNAP-tag (a 20 kDa mutant of the DNA repair protein O6-alkylguanine-DNA alkyltransferase) forms a covalent bond with benzylguanine (BG) derivatives.(28) BG could thus be used to label the RT primer in place of the HaloLigand. A mutant derivative of the SNAP-tag, the CLIP-tag, binds O2-benzylcytosine (BC) derivatives, which could also be adapted to MIPSA.(29)

The rate of fusion tag maturation and ligand binding is important to the relative yield of cis versus trans bonds. A study by Samelson et al. determined that the rate of Halo Tag protein production is about four-fold higher than the rate of HaloTag functional maturation.(30) Considering a typical protein size is <1,000 amino acids in the ORFeome library, these data predict that most proteins would be released from the ribosome before HaloTag maturation and thus before cis HaloLigand binding could occur, thereby favoring unwanted trans barcoding. During optimization experiments, the present inventors found the rate of cis barcoding to be slightly improved by excluding release factors from the translation mix, which stalls ribosomes on their native ORF stop codons. HaloTag maturation thus continues while remaining in proximity to the cis HaloLigand-conjugated primer. Alternative approaches to promote controlled ribosomal stalling could also include stop codon removal/suppression or use of a dominant negative release factor. Ribosome release could then be accomplished via addition of the chain terminator puromycin.

Because UCIs are formed on the 5′ UTR of the mRNA, eukaryotic ribosomes would be unable to scan from the 5′ cap to the initiating Kozak sequence. In cases in which cap-dependent translation is required, two alternative methods could be employed. First, the current 5′ UCI system could be used if an internal ribosome entry site (IRES) were to be placed between the RT primer and the Kozak sequence. Second, the UCI could instead be situated at the 3′ end of the mRNA, provided that the RT was prevented from extending into the ORF. Beyond cell-free translation, if either of these approaches were developed, mRNA-cDNA hybrids could be transfected into living cells or tissues, where UCI-protein formation could take place in situ.

The ORF-associated UCIs can be embodied in a variety of ways. In particular embodiments, and as described in the Examples section, the present inventors have stochastically assigned indexes to the human ORFeome at ˜10× representation. This approach has two main benefits, first being the low cost of the synthetic oligonucleotide library (a single degenerate oligonucleotide pool), and second being the multiple, independent pieces of evidence reported by the set of UCIs associated with each ORF. In certain embodiments, the library of stochastic barcodes is designed to feature sequences of uniform melting temperature, and thus uniform PCR amplification efficiency. For simplicity, the present inventors have opted not to incorporate unique molecular identifiers (UMIs) into the primer, but this approach is compatible with MIPSA UCIs, and may potentially enhance quantitation. One disadvantage of stochastic indexing is the potential for ORF dropout, and thus the need for relatively high UCI representation; this increases the depth of sequencing required to quantify each UCI, and thus the overall per-sample cost. A second disadvantage is the requirement to construct a UCI-ORFeome matching dictionary. With short-read sequencing, the present inventors were unable to disambiguate a fraction of the library, comprised mostly of alternative isoforms. Using a long-read sequencing technology, such as PacBio or Oxford Nanopore Technologies, instead of, or in addition to short read technology could surmount incomplete disambiguation during UCI-ORF matching. As opposed to stochastic barcoding, individual ORF-UCI cloning is possible but costly and cumbersome. However, a smaller UCI set would provide the advantage of lower per-assay sequencing cost. The present inventors have previously developed a methodology to clone ORFeomes using Long Adapter Single Stranded Oligonucleotide (LASSO) probes.(31) Incorporating target-specific indexes into the capture probe library would result in uniquely indexed ORFs, without dramatically increasing the cost of the LASSO probe library. LASSO cloning of ORFeome libraries may therefore synergize with MIPSA-based applications.

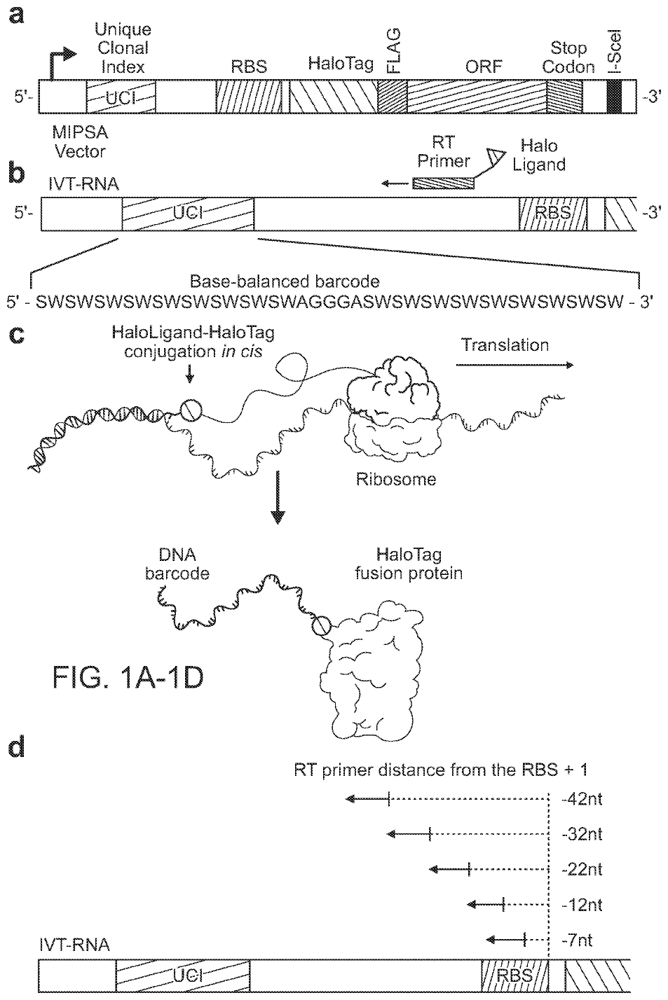

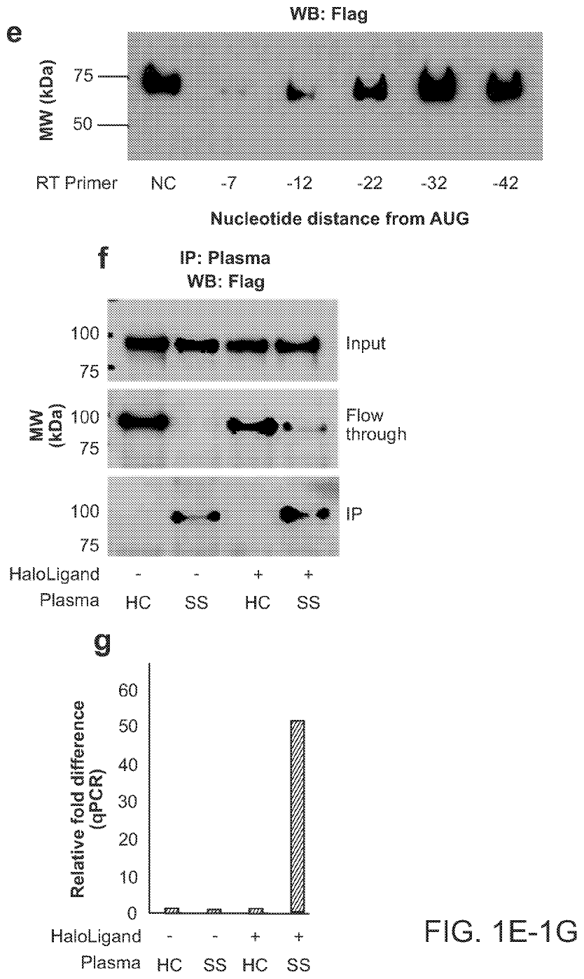

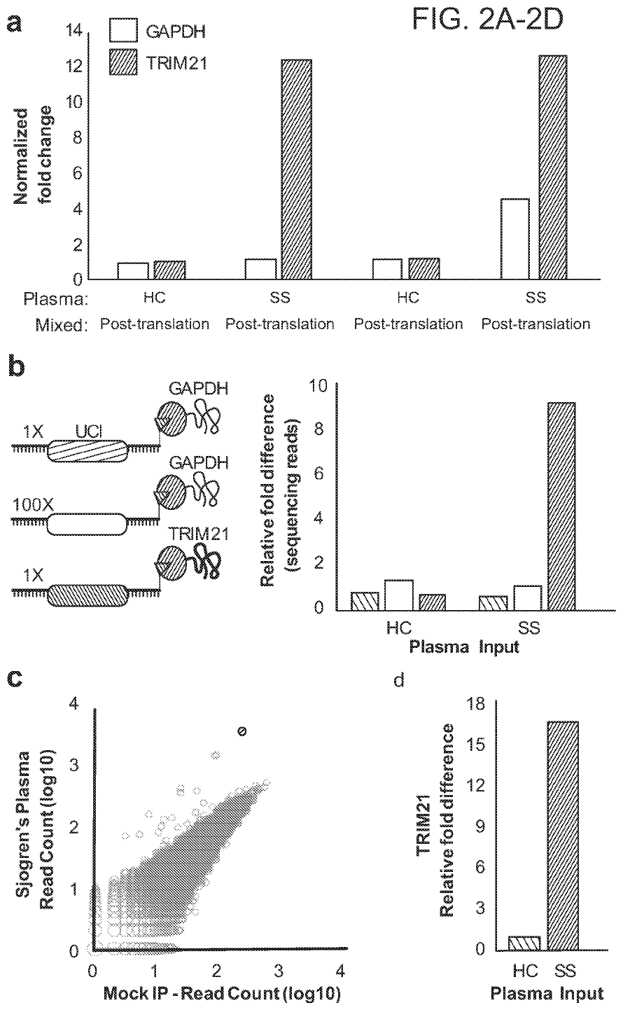

MIPSA readout via qPCR. A useful feature of appropriately designed UCIs is that they can also serve as qPCR readout probes. The degenerate UCIs that the present inventors have designed and used here (FIG. 1B) also comprise 18 nt Tm balanced forward and reverse primer binding sites. The low cost and rapid turnaround time of a qPCR assay can thus be leveraged in combination with MIPSA. For example, incorporating assay quality control measures, such as the TRIM21 IP, can be used to qualify a set of samples prior to a more costly sequencing run. Troubleshooting and optimization can similarly be expedited by employing qPCR as a readout, rather than NGS. qPCR testing of specific UCIs may theoretically also provide enhanced sensitivity compared to sequencing, and may be more amenable to analysis in a clinical setting.

### I. Definitions

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, β-amino acids, homo-amino acids, proline and pyruvic acid derivatives, 3-substituted alanine derivatives, glycine derivatives, ring-substituted phenylalanine and tyrosine derivatives, linear core amino acids, N-methyl amino acids.

As used herein, the term “polypeptide” encompasses peptides and proteins, and refers to a molecule comprising a chain of two or more amino acids joined by peptide bonds. In some embodiments, a polypeptide comprises 2 to 50 amino acids, e.g., having more than 20-30 amino acids. In some embodiments, a peptide does not comprise a secondary, territory, or higher structure. In some embodiments, a protein comprises 30 or more amino acids, e.g. having more than 50 amino acids. In some embodiments, in addition to a primary structure, a protein comprises a secondary, territory, or higher structure. The amino acids of the polypeptide are most typically L-amino acids, but may also be D-amino acids, unnatural amino acids, modified amino acids, amino acid analogs, amino acid mimetics, or any combination thereof. Polypeptides may be naturally occurring, synthetically produced, or recombinantly expressed. Polypeptide may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “proteome” can include the entire set of proteins, polypeptides, or peptides (including conjugates or complexes thereof) expressed by a target, e.g., a genome, cell, tissue, or organism at a certain time, of any organism. In one aspect, it is the set of expressed proteins in a given type of cell or organism, at a given time, under defined conditions. Proteomics is the study of a proteome. For example, a “cellular proteome” may include the collection of proteins found in a particular cell type under a particular set of environmental conditions, such as exposure to hormone stimulation. An organism's complete proteome may include the complete set of proteins from all of the various cellular proteomes. A proteome may also include the collection of proteins in certain sub-cellular biological systems. For example, all of the proteins in a virus can be called a viral proteome. As used herein, the term “proteome” include subsets of a proteome, including but not limited to a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any combination thereof.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide.

As used herein, the term “barcode” refers to a nucleic acid molecule of about 2 to about 10 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 bases) providing a unique identifier tag or origin information for a macromolecule, each macromolecule in a library of macromolecules, and the like. A barcode can be an artificial sequence or a naturally occurring sequence. The concept of the barcode is that prior to any amplification, each original target molecule is “tagged” by a unique barcode sequence. In some embodiments, the DNA sequence must be long enough to provide sufficient permutations to assign each founder molecule a unique barcode.

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. The term “forward” when used in context with a “universal priming site” or “universal primer” may also be referred to as “5′” or “sense.” The term “reverse” when used in context with a “universal priming site” or “universal primer” may also be referred to as “3” or “antisense.”

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays.

The terms “specifically binds to,” “specific for,” and related grammatical variants refer to that binding which occurs between such paired species as ligand/tag, antibody/antigen, aptamer/target, enzyme/substrate, receptor/agonist and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, in certain embodiments, “specific binding” occurs between a paired species where there is interaction between the two which produces a bound complex having the characteristics of, for example, an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M. In certain embodiments, the term refers to a molecule (e.g., an aptamer) that binds to a target (e.g., a protein) with at least five-fold greater affinity as compared to any non-targets, e.g., at least 10-, 20-, 50-, or 100-fold greater affinity. In particular embodiments, a polypeptide tag specifically binds to its ligand. In specific embodiments, a polypeptide tag covalently binds to a ligand.

A “biological sample,” as used herein, is generally a sample from an individual or subject. Non-limiting examples of biological samples include blood, serum, plasma, or cerebrospinal fluid. Additionally, solid tissues, for example, spinal cord or brain biopsies may be used.

### II. Vectors, Libraries Thereof and Methods of Using the Same

The present disclosure provides vectors and self-assembled protein display libraries comprising of plurality of vectors. In specific embodiments, a vector comprises a nucleic acid sequence that encodes a protein of interest. In one embodiment, a vector comprises along the 5′ to 3′ direction (a) a polymerase transcriptional start site; (b) a barcode; (c) a reverse transcription primer binding site; (d) a RBS; and (e) a nucleotide sequence encoding a fusion protein comprising (i) a polypeptide tag and (ii) a protein of interest, wherein the polypeptide tag specifically binds a ligand.

In particular embodiments, the vector further comprises an endonuclease site for vector linearization. In other embodiments, the vector further comprises (vii) a stop codon.

In a specific embodiment, the barcode is flanked by binding sites for polymerase chain reaction (PCR) primers. In an alternative embodiment, the barcode comprises binding sites for PCR primers.

In another embodiment, the RBS comprises an internal ribosome entry site.

In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes are different.

A population of barcodes may be randomly generated or non-randomly generated. In some embodiments, a barcode contains randomized nucleotides and is incorporated into a nucleic acid. For example, a 12-base random sequence provides 412 or 16,777,216 UMI's for each target molecule in the sample.

In particular embodiments, barcodes can be used to computationally deconvolute multiplexed sequencing data and identify sequence derived from an individual macromolecule, sample, library, etc.

The present disclosure also provides methods for using the self-assembled protein display libraries. In certain embodiments, a method comprises the steps of (a) transcribing a linearized or nicked plurality of vectors comprising a self-assembled protein display library to produce mRNA; (b) reverse transcribing the 5′ end of the mRNA to produce cDNA comprising the barcodes using a primer conjugated to the ligand; and (c) translating the mRNA, wherein the polypeptide tag of the fusion protein covalently binds the ligand conjugated to the cDNA comprising the barcode.

In a more specific embodiment, a method comprises the steps of (a) transcribing a vector library into messenger ribonucleic acid (mRNA), wherein the vector library encodes a plurality of proteins, and wherein each vector of the vector library comprises in the 5′ to 3′ direction: (i) a polymerase transcriptional start site; (ii) a barcode; (iii) a reverse transcription primer binding site; (iv) a ribosome binding site (RBS); and (v) a nucleotide sequence encoding a fusion protein comprising (1) a polypeptide tag and (2) a protein, wherein the polypeptide tag specifically binds a ligand; (b) reverse transcribing the 5′ end of the mRNA using a primer that binds upstream of the RBS, wherein the primer is conjugated with the ligand that specifically binds the polypeptide tag of the fusion protein, and wherein a complementary deoxyribonucleic acid (cDNA) is formed comprising the ligand, primer and barcode; and (c) translating the mRNA, wherein the ligand of the cDNA binds the polypeptide tag of the fusion protein. In a specific embodiment, the vector library is nicked prior to step (a). In another specific embodiment, the vector further comprises (vi) an endonuclease site for vector linearization and the vector library is linearized prior to step (a).

### III. Self-Assembled Protein-DNA Conjugates, Libraries Thereof and Methods of Using the Same

The present disclosure also provides a self-assembled protein-DNA conjugate composition and libraries comprising the same. In particular embodiments, each protein-DNA conjugate comprises (a) a cDNA comprising a barcode, wherein the cDNA is conjugated with a ligand that specifically binds a polypeptide tag; and (b) a fusion protein comprising the polypeptide tag and a protein of interest, wherein the ligand is covalently bound to the polypeptide tag.

In certain embodiments, more than one copy of a protein of interest can be present as a protein-DNA conjugate in a library of protein-DNA conjugates and each copy of the protein of interest can comprise a unique barcode.

In particular embodiments, the polypeptide tag is fused to the N-terminal end of the protein of interest. In other embodiments, the polypeptide tag is fused to the C-terminal end of the protein of interest.

In certain embodiments, the polypeptide tag comprises haloalkane dehalogenase or O⁶-alkylguanine-DNA-alkyltransferase. In a specific embodiment, the polypeptide tag comprises a HALO-tag and the ligand comprises a HALO-ligand. In a more specific embodiment, the HALO-tag comprises the amino acid sequence set forth in SEQ ID NO:22. In other embodiments, the HALO-ligand comprises one of:

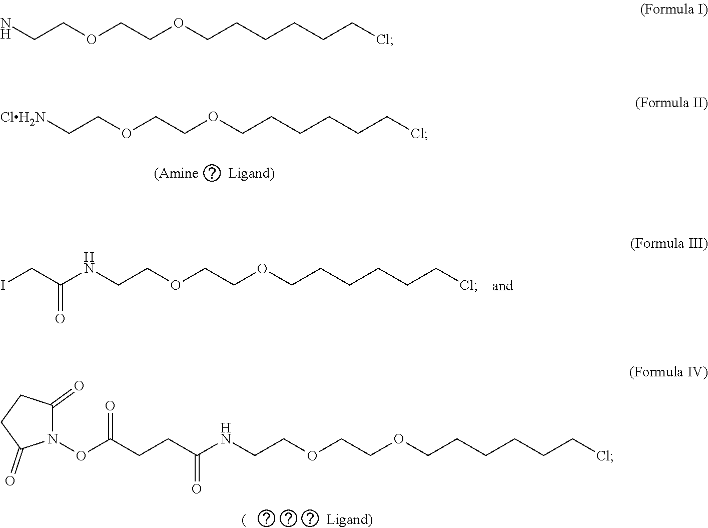

HALOTAG® tags and ligands are available commercially from Promega (Madison, Wis.) and are conjugated with nucleic acids according to the manufacturer's instructions. In a specific embodiment, to conjugate a HALOTAG® ligand to a DNA sequence (e.g., a reverse transcription primer), the DNA sequence is modified with an alkyne group. The azido halo ligand is then reacted with the alkyne terminated DNA sequence using the Cu-catalyzed cycloaddition (“click” chemistry). See, e.g., Duckworth et al. 46 ANGEW CHEM. INT. 8819-22 (2007).

Alternatively, other polypeptide tag-ligand capture moiety systems can be used. For example, O6-alkylguanine-DNA alkyltransferase, reacts specifically and rapidly with benzylguanine (BG) and derivatives thereof. In a specific embodiment, the polypeptide tag comprises SNAP-TAG® (New England Biolabs (Ipwich, MA)). SNAP-TAG® is a self-labeling protein derived from human O⁶-alkylguanine-DNA-alkyltransferase. SNAP-TAG® reacts with covalently with O⁶-benzylguanine derivatives. In one embodiment, the polypeptide tag comprises the amino acid sequence set forth in SEQ ID NO:23. In another specific embodiment, the polypeptide tag comprises CLIP-TAG (New England Biolabs), which is a modified version of SNAP-TAG®. It is also a self-labeling protein derived from human O⁶-alkylguanine-DNA-alkyltransferase. Instead of benzylguanine derivatives, CLIP tag is engineered to react with benzylcytosine derivatives. In a specific embodiment, the polypeptide tag comprises the amino acid sequence set forth in SEQ ID NO:24. See Keppler et al. 1 NAT BIOTECHNOL. 86-99 (2003); and Gautier et al. 15 (2) CHEM. BIOL. 128-36 (2008).

The present disclosure also provides methods for using the library of self-assembled protein-DNA conjugates. In one embodiment, a method for studying protein-protein interactions comprises the step of performing a pull-down assay of the library of protein-DNA conjugates with a protein of interest. In another embodiment, a method for studying protein-small molecule interactions comprises the step of performing a pull-down assay of the library of protein-DNA conjugates with a small molecule. In yet another embodiment, a method comprises the step of performing an immunoprecipitation of the library of protein-DNA conjugates with antibodies obtained from a biological sample. In a further embodiment, a method for identifying the target of a first small molecule comprises the steps of (a) incubating the library of protein-DNA conjugates with the first small molecule that binds its target(s) and (b) performing a pull-down assay of the library of step (a) with a second small molecule, wherein the first small molecule bound to its target(s) blocks the binding of the second small molecule. In a more specific embodiment, more than one small molecule is used in the pull-down assay of step (b).

### IV. Treatment of COVID-19

The present disclosure also provides methods for treating COVID-19. In one embodiment, a method for treating a patient having severe COVID-19 comprises the step of administering to the patient an effective amount of interferon therapy, wherein autoantibodies that neutralize IFN-λ3 are detected in a biological sample obtained from the patient. In another embodiment, a method for treating a patient having severe COVID-19 comprises the steps of (a) detecting autoantibodies that neutralize IFN-λ3 in a biological sample obtained from the patient; and (b) treating the patient with an effective amount of interferon therapy. In a further embodiment, a method for identifying a COVID-19 patient who would benefit from interferon therapy comprises the step of detecting autoantibodies that neutralize IFN-λ3 in a biological sample obtained from the patient. In particular embodiments, the interferon therapy comprises interferon lambda (IFN-λ) or interferon beta (IFN-β). In specific embodiments, interferon lambda (IFN-λ) or interferon beta (IFN-β) is pegylated. In a further embodiment, the interferon therapy comprises interferon omega (IFN-ω).

The terms “interferon”, “IFN” and “interferon molecule” are used herein interchangeably. They refer to any interferon or interferon derivative (e.g., pegylated interferon) that can be used in the treatment of COVID-19.

Interferons are a family of cytokines produced by eukaryotic cells in response to viral infection and other antigenic stimuli, which display broad-spectrum antiviral, antiproliferative and immunomodulatory effects. Recombinant forms of interferons have been widely applied in the treatment of various conditions and diseases, such as viral infections (e.g., HCV, HBV and HIV), inflammatory disorders and diseases (e.g., multiple sclerosis, arthritis, cystic fibrosis), and tumors (e.g., liver cancer, lymphomas, myelomas, etc.).

Interferons are classified as Type I, Type II and Type III, depending on the cell receptor to which they bind. Type I interferons bind to a specific cell surface receptor complex known as the IFN-alpha (IFN-α) receptor (IFNAR) that consists of two chains (IFNAR1 and IFNAR2). The type I interferons present in humans are interferon-alpha (IFN-α), interferon-beta (IFN-β) and interferon-omega (IFN-ω).

Type III interferons signal through a receptor complex consisting of the interferon-lambda receptor (IFNLR1 or CRF2-12) and the interleukin 10 receptor 2 (IL10R2 or CRF2-4). In humans, type III interferons include three interferon lambda (IFN-λ) proteins referred to as IFN-λ1, IFN-λ2 and IFN-λ3 also known as interleukin 29 (IL-29), interleukin 28A (IL-28A) and interleukin 28B (IL-28B), respectively.

Therefore, in certain embodiments, interferon therapy comprises one or more of IFN-α, IFN-β, IFN-ω, IFN-γ, IFN-λ, analogs thereof and derivatives thereof. In certain embodiments, interferon therapy comprises IFN-λ, analogs thereof and derivatives thereof. In other embodiments, interferon therapy comprises IFN-β, analogs thereof and derivatives thereof.

As used herein, the terms “interferon”, “IFN and “IFN molecule” more specifically refer to a peptide or protein having an amino acid substantially identical (e.g., et least 70%, 75%, 80%, 85%, 90%, 95%, 96%, 97%, 98%, 99% or even 100% identical) to all or a portion of the sequence of an interferon (e.g., a human interferon), such as IFN-α, IFN-β, IFN-ω, IFN-γ, and IFN-λ that are known in the art. Interferons suitable for use in the present disclosure include, but are not limited to, natural human interferons produced using human cells, recombinant human interferons produced from mammalian cells, E-coli-produced recombinant human interferons, synthetic versions of human interferons and equivalents thereof. Other suitable interferons include consensus interferons which are a type of synthetic interferons having an amino acid sequence that is a rough average of the sequence of all the known human IFN subtypes (for example, all the known IFN-β subtypes, or all the known IFN-λ subtypes.

The terms “interferon”, “IFN”, and “IFN molecule” also include interferon derivatives, i.e., molecules of interferon (as described above) that have been modified or transformed. A suitable transformation may be any modification that imparts a desirable property to the interferon molecule. Examples of desirable properties include, but are not limited to, prolongation of in vivo half-life, improvement of therapeutic efficacy, decrease of dosing frequency, increase of solubility/water solubility, increase of resistance against proteolysis, facilitation of controlled release, and the like. As mentioned above, pegylated interferons have been produced (e.g., pegylated IFN-λ) and are currently used to treat hepatitis. Pegylated interferons exhibit longer half-lives, which allows for less frequent administration of the drug. Pegylating an interferon molecule involves covalently binding the interferon to polyethylene glycol (PEG), an inert, non-toxic and biodegradable organic polymer. Therefore, in certain embodiments, interferon therapy comprises a pegylated interferon. Interferons have also been produced as fusion proteins with human albumin (e.g., albumin-IFN-λ). The albumin-fusion platform takes advantage of the long half-life of human albumin to provide a treatment that allows the dosing frequency of IFN to be reduced. Therefore, in certain embodiments, interferon therapy comprises an albumin-interferon fusion protein.

The present disclosure provides methods for detecting autoantibodies to IFN-λ3. In more specific embodiments, autoantibodies that neutralize IFN-λ3 are detected. The presence of autoantibodies that neutralize IFN-λ3 can be used to identify COVID-19 patients who would benefit from interferon therapy. In particular embodiments, the patient has severe COVID-10. Inteferon therapy can be administered to COVID-19 patients wherein autoantibodies that neutralize IFN-λ3 have been detected in biological sample obtained from the patient.

IFN-λ3 polypeptides can be used in an immunoassay to detect IFN-λ3-specific autoantibodies in a biological sample. IFNλ-3 polypeptides used in an immunoassay can be in a cell lysate (e.g., a whole cell lysate or a cell fraction), or purified IFNλ-3 polypeptides or fragments thereof can be used provided at least one antigenic site recognized by IFNλ-3-specific autoantibodies remains available for binding. Depending on the nature of the sample, either or both immunoassays and immunocytochemical staining techniques may be used. Enzyme-linked immunosorbent assays (ELISA), Western blot, and radioimmunoassays can be used as described herein to detect the presence of IFNλ-3-specific autoantibodies in a biological sample.

IFNλ-3 polypeptides or fragments thereof may be used with or without modification for the detection of IFNλ-3-specific autoantibodies. Polypeptides can be labeled by either covalently or non-covalently combining the polypeptide with a second substance that provides for detectable signal. A wide variety of labels and conjugation techniques can be used. Some examples of labels that can be used include radioisotopes, enzymes, substrates, cofactors, inhibitors, fluorescers, chemiluminescers, magnetic particles, and the like

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their disclosure. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Molecular Indexing of Proteins by Self Assembly (MIPSA) for Efficient Proteomic Investigations

### Materials and Methods

MIPSA destination vector construction and UCI barcode library construction. The MIPSA vector was constructed using the pDEST15 vector as a backbone. A gBlock fragment (Integrated DNA Technologies) encoding the RBS, Kozak sequence, N-terminal HaloTag fusion protein, FLAG tag, and attR1 sequence was cloned into the parent plasmid. A 150 bp poly(A) sequence was also added after attR2 and stop codon. A 41 nt barcode oligo was generated within a gBlock Gene Fragment (Integrated DNA Technologies) with alternating mixed bases (S: G/C; W: A/T) to produce the following sequence: (SW)₁₈-AGGGA-(SW)₁₈. The sequences flanking the degenerate barcode incorporated the standard PhIP-Seq PCR1 and PCR2 primer binding sites. (43) 18 ng of the starting UCI library was used to run 40 cycles of PCR to amplify the library and incorporate BglII and PspxI restriction sites. The MIPSA vector and amplified UCI library were then digested with the restriction enzymes overnight, column purified, and ligated at 1:5 vector-to-insert ratio. The ligated MIPSA vector was used to transform electrocompetent One Shot ccdB 2 T1^{R} cells (Thermo Fisher Scientific). 6 transformation reactions yielded ˜800,000 colonies to produce the pDEST-MIPSA UCI library.

Human ORFeome recombination into barcoded MIPSA vector. 150 ng of the pENTR-hORFeome-(L1-L5) vector was combined with 150 ng of the pDEST-MIPSA vector and 2 μL of Gateway LR Clonase II mix (Life Technologies) for a total reaction volume of 10 μL. The reaction was incubated overnight at 25° C. The entire reaction was transformed into 50 μL of One Shot OmniMAX 2 T1^{R} chemical competent E. coli (Life Technologies). Transformation yielded ˜120,000 colonies, which is ˜10× of each human subpool library. Colonies were collected and pooled by scraping, followed by purification of the barcoded-pDEST-MIPSA-hsORFeome plasmid DNA (human ORFeome MIPSA library) using the Qiagen Plasmid Midi Kit (Qiagen).

HaloLigand conjugation to RT oligo and HPLC purification. 100 μg of a 5′ amine modified oligo (Table 1) was incubated with 75 μL (17.85 μg/μL) of the Succinimidyl Ester (O2) HaloLigand (Promega Corporation) in 0.1 M sodium borate buffer for 6 hours at room temperature following Gu et al.(14) Three M NaCl and ice-cold ethanol was added at 10% (v/v) and 250% (v/v), respectively, to the labeling reaction and incubate overnight at −80° C. The reaction was centrifuged for 30 minutes at 12,000×g. The pellet was rinsed once in ice-cold 70% ethanol and air-dried for 10 minutes.

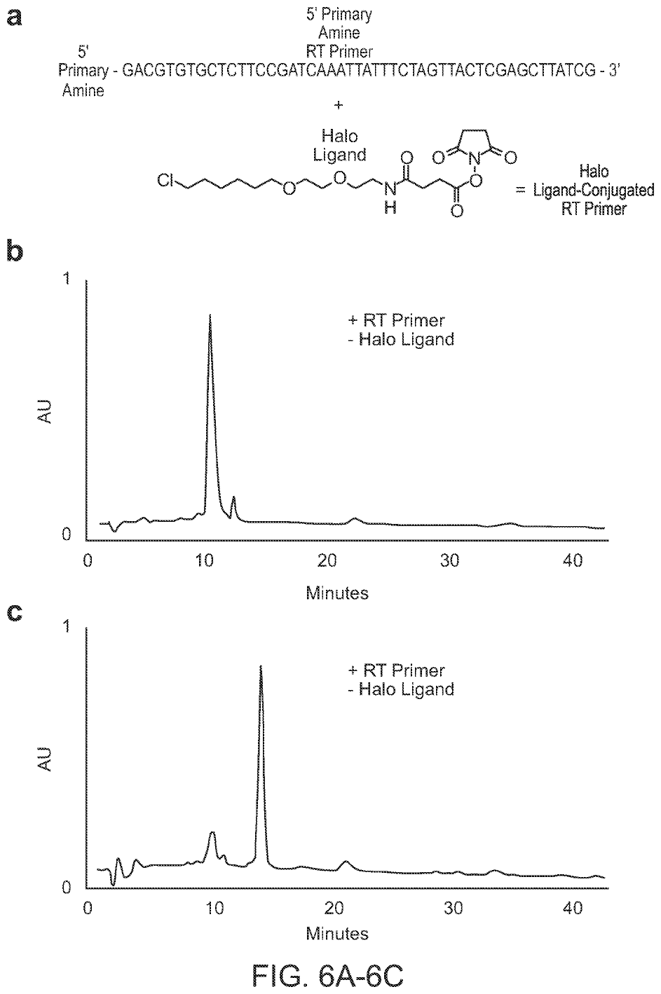

HaloLigand-conjugated RT primer was HPLC purified using a Brownlee Aquapore RP-300 7u, 100×4.6 mm column (Perkin Elmer) using a two-buffer gradient of 0-70% CH3CN/MeCN (100 mM triethylamine acetate to acetonitrile) over 70 minutes. Fractions corresponding to labeled oligo were collected and lyophilized (FIG. 6). Oligos were resuspended at 1 μM and stored at −80° C.

MIPSA RNA library preparation. The pDEST-MIPSA vector containing the human ORFeome library (4 μg) was linearized with the I-SceI restriction endonuclease (New England Biolabs) overnight. The product was column-purified with the NucleoSpin Gel and PCR Clean Up kit (Macherey-Nagel GmbH & Co. KG). A 40 μL HiScribe T7 High Yield RNA Synthesis Kit (New England Biolabs) was utilized to transcribe 1 μg of the purified, linearized product. The product was diluted with 60 μL molecular biology grade water, and 1 μL of DNAse I was added. The reaction was incubated for another 15 minutes at 37° C. Then 50 μL of 1 M LiCl was added to the solution and incubated at −80° C. overnight. A centrifuge was cooled to 4° C., and the RNA was spun at max speed for 30 minutes. The supernatant was removed, and the RNA pellet washed with 70% ethanol. The sample was spun down at 4° C. for another 10 minutes, and the 70% ethanol removed. The pellet was dried at room temperature for 15 minutes, and subsequently resuspended in 100 μL water. To preserve the sample, 1 μL of 40 U/μL RNAseOUT Recombinant Ribonuclease Inhibitor (Life Technologies, Carlsbad CA) was added.

MIPSA RNA library reverse transcription and translation. A reverse transcription reaction was prepared using SuperScript IV First-Strand Synthesis System (Life Technologies). First, 1 μL of 10 mM dNTPs, 1 μL of RNAseOUT (40 U/μL), 4.17 μL of the RNA library (1.5 μM), and 7.83 μL of the HaloLigand-conjugated RT primer (1 μM, Table 1) was combined for a single 14 μL reaction and incubated at 65° C. for 5 minutes followed by a 2-minute incubation on ice. 4 μL of 5×RT buffer, 1 μL of 0.1 M DTT, and 1 μL of SuperScript IV RT Enzyme (200 U/μL) was added to the 14 μL reaction on ice and incubated for 20 minutes at 42° C. A single 20 μL RT reaction received 36 μL of RNAClean XP beads (Beckman Coulter), and was incubated at room temperature for 10 minutes. The beads were collected by magnet and washed five times with 70% ethanol. The beads were air-dried for 10 minutes at room temperature and resuspended in 7 μL of 5 mM Tris-HCl, pH 8.5. The product (2 μL) was analyzed with spectrophotometry to measure the RNA yield. A translation reaction was set up on ice using the PURExpress Δ Ribosome Kit (New England Biolabs).(44) The reaction was modified such that the final concentration of ribosomes was 0.3 μM. 4.57 μL of the RT reaction was added to 4 μL Solution A, 1.2 μL Factor Mix, and 0.23 μL ribosomes (13.3 μM). This reaction was incubated at 37° C. for two hours, diluted to a total volume of 45 μL with 35 μL 1×PBS, and used immediately or stored at −80° C. after addition of 25% glycerol. In optimization experiments utilizing the PURExpress Δ RF123 Kit (New England Biolabs), Solution B was substituted with NEB custom-made Factor Mix (-RF123, -ribosomes). Following the incubation step at 37° C. for two hours, either RNase A was added, or release factors 1, 2, and 3 were added, and the reaction proceeded on ice for 30 minutes.

Immunoprecipitation using MIPSA library. 5 μL of serum is mixed with the 45 μL of diluted MIPSA library (see above) and incubated overnight at 4° C. with gentle agitation. For each IP, a mixture of 5 μL of Protein A Dynabeads and 5 μL of Protein G Dynabeads (Life Technologies) was washed 3 times in 2× their original volume with 1×PBS. The beads were then resuspended in 1×PBS at their original volume, and added to each IP. The binding proceeded for 4 hours at 4° C. The beads were collected on a magnet and the beads were washed 3 times in 1×PBS, changing tubes or plates between washes. The beads were then collected and resuspended in a 20 μL PCR master mix containing the T7-Pep 2 PCR1 F forward and the T7-Peps PCR1 R+ad min reverse primers (Table 1) and Herculase-II (Agilent). PCR cycling was as follows: an initial denaturing step at 95° C. for 2 min, followed by 30 cycles of: 95° C. for 20 s, 58° C. for 30 s, 72° C. for 30 s, with a final extension of 72° C. for 3 min. Two microliters of the amplification product were used as input to a 20 μL dual-indexing PCR reaction for 10 cycles with the PhIP PCR2 F forward and the Ad min BCX P7 reverse primers. PCR cycling was as follows: an initial denaturing step at 95° C. for 2 min, followed by 10 cycles of: 95° C. for 20 s, 58° C. for 30 s, 72° C. for 30 s, with a final extension of 72° C. for 3 min. i5/i7 indexed libraries were pooled and column purified. Libraries were sequenced on an Illumina NextSeq 500 using a 1×75 nt protocol. Plato2_i5_NextSeq_SP and Standard_i7_SP primers were used for i5/i7 identification (Table 1). The output was demultiplexed using i5 and i7 without allowing any mismatches.

Phage ImmunoPrecipitation Sequencing. The design and cloning of the 90 amino acid human peptidome library was previously described.(24) Phage immunoprecipitation and sequencing was performed according to our published protocol.(45) Briefly, 0.2 μl of each plasma was individually mixed with the human phage library and then immunoprecipitated using protein A and protein G coated magnetic beads. A set of 8 mock IPs were run on each 96 well plate. Amplicons were sequenced on an Illumina NextSeq 500 instrument.

For quantification of MIPSA experiments by qPCR, the PCR1 product was analyzed as follows. A 4.6 μL of 1/1000 dilution of the PCR1 reaction was resuspended in a 10 μL qPCR master mix containing 5 μL of Brilliant III Ultra Fast 2×SYBR Green Mix (Agilent), 0.2 μL of 2 μM reference dye and 0.2 μL of 10 μM forward and reverse primer mix (specific to the target UCI). PCR cycling was as follows: an initial denaturing step at 95° C. for 2 min, followed by 30 cycles of: 95° C. for 20 s, 60° C. for 30 s for 45 cycles. Following completion of thermocycling, amplified products were subjected to melt-curve analysis. The qPCR primers for MIPSA immunoprecipitation experiments are as follows: BT2_F and BT2_R for TRIM21, BG4_F and BG4_R for GAPDH, and NT5C1A_F and NT5C1A_R for NT5C1A (Table 1).

Plasma Samples. All samples were collected by the studies where the subjects met protocol eligibility criteria, as described below. All of the studies protected the rights and privacy of the study participants and were approved by their respective Intuitional Review Boards for original sample collection and subsequent analyses.

Pre-pandemic plasma samples. All human samples were collected prior to 2017 at the National Institutes of Health (NIH) Clinical Center under the Vaccine Research Center's (VRC)/National Institutes of Allergy and Infectious Diseases (NIAID)/NIH protocol “VRC 000: Screening Subjects for HIV Vaccine Research Studies” (NCT00031304) in compliance with NIAID IRB approved procedures.

COVID-19 Convalescent Plasma (CCP) from non-hospitalized patients. Eligible CCP donors were contacted by study personnel, as previously described. (46,47) All donors were at least 18 years old and had a confirmed diagnosis of SARS-CoV-2 by detection of RNA in a nasopharyngeal swab sample. Basic demographic information (age, sex, hospitalization with COVID-19) was obtained from each donor; initial diagnosis of SARS-CoV-2 and the date of diagnosis were confirmed by medical chart review. Samples were separated into plasma and peripheral blood mononuclear cells within 12 hours of collection, and the plasma samples were immediately frozen at −80° C.

Severe COVID-19 plasma samples. The study cohort was defined as inpatients who had: 1) a confirmed diagnosis of COVID-19; 2) survival to death or discharge; and 3) remnant specimens in the Johns Hopkins COVID-19 Remnant Specimen Biorepository, an opportunity sample that includes 59% of Johns Hopkins Hospital COVID-19 patients and 66% of patients with length of stay >=3 days. (48) Selection and frequency of other laboratory testing were determined by treating physicians. Patient outcomes were defined by the World Health Organization (WHO) COVID-19 disease severity scale. Samples from severe COVID-19 patients that were included in this study were obtained from 17 patients who died, 13 who recovered after being ventilated, 22 who required oxygen to recover, and 3 who recovered without supplementary oxygen. This study was approved by the JHU Institutional Review Board (IRB00248332, IRB00273516), with a waiver of consent because all specimens and clinical data were de-identified by the Core for Clinical Research Data Acquisition of the Johns Hopkins Institute for Clinical and Translational Research; the study team had no access to identifiable patient data.

Sjögren's syndrome samples were collected under protocol NA_00013201. All patients were >18 years old and gave informed consent. IBM patient samples were collected under protocol IRB00235256. All patients met ENMC 2011 diagnostic criteria (49) and provided informed consent.

Immunoblot analysis. Laemmli buffer containing 5% β-ME was added to post-translation samples, boiled for 5 min, and analyzed on NuPage 4-12% Bis-Tris polyacrylamide gels (Life Technologies). Following transfer to PVDF membranes, blots were blocked in 20 mM Tris-buffered saline, pH 7.6, containing 0.1% Tween 20 (TBST) and 5% (wt/vol) non-fat dry milk for >1 hour at room temperature. Blots were subsequently incubated overnight at 4° C. with primary antibodies followed by 4-hour incubations at room temperature in secondary antibodies.

Construction of the UCI-ORF dictionary. The Nextera XT DNA Library Preparation kit (Illumina) was used for tagmentation of 150 ng of each library to yield the optimal size distribution centered around 1.5 kb. Tagmented MIPSA human ORFeome libraries were amplified using Herculase-II (Agilent) with T7-Pep2 PCR1 F forward and a Nextera Index 1 Read primer. PCR cycling was as follows: an initial denaturing step at 95° C. for 2 min, followed by 30 cycles of: 95° C. for 20 s, 53.5° C. for 30 s, 72° C. for 30 s, with a final extension of 72° C. for 3 min. PCR reactions were run on a 1% agarose gel followed by excision of ˜1.5 kb products and purification using the NucleoSpin Gel & PCR Clean-up columns (Mackery Nagel). The purified product was then amplified for another 10 cycles with the PhIP PCR2 F forward primer and P7.2 reverse primers (see Table 1 for list of primer sequences). The product was gel-purified and sequenced on a MiSeq (Illumina) using the T7-Pep2.2 SP subA primer for read 1 and the MISEQ PLATO R2 primer for read 2. Read 1 was 60 bp long to capture the UCIs. The first index read, I1, was substituted with a 50 bp read into the ORF. I2 was used to identify the i5 index for sample demultiplexing.

The human ORFeome V8.1 DNA sequences were truncated to the first 50 nt, and the ORF names corresponding to non-unique sequences were concatenated. The demultiplexed output of the 50 nt R2 (ORF) read from an Illumina MiSeq was aligned to the truncated human ORFeome V8.1 library using the Rbowtie2 package (50) with the following parameters: options=“-a --very-sensitive-local”. The unique FASTQ identifiers were then used to extract corresponding sequences from the 60 bp R1 (UCI) read. Those sequences were then truncated using the 3′ anchor ACGATA, and sequences that did not have the anchor were removed. Additionally, any truncated R1 sequences that had fewer than 18 nucleotides were removed. The ORF sequences that still had a corresponding UCI post-filtering were retained using the FASTQ identifier. The names of ORFs that had the same UCI were then concatenated, and this final dictionary was used to generate a FASTA alignment file with ORF names and UCI sequences.

Informatic analysis of MIPSA data. Illumina output FASTQ files were truncated using the constant ACGAT anchor sequence following all UCI sequences. Next, perfect match alignment was used to map the truncated sequences to their linked ORFs via the UCI-ORF lookup dictionary. A counts matrix is constructed, in which rows correspond to individual UCIs and columns correspond to samples. The present inventors next used the edgeR software package (51) which, using a negative binomial model, compares the signal detected in each sample against a set of negative control (“mock”) IPs that were performed without serum, returning a fold change value and a test statistic for each UCI in every sample, thus creating fold-change and significance matrices. Significantly enriched UCIs (“hits”), required a read count of at least 15, a p-value less than 0.001, and a fold changes of at least 3. Hits fold-change matrices report the fold change value for “hits” and report a “1” for UCIs that are not hits.

Protein sequence similarity. To evaluate sequence homology among proteins in the hORFeome v8.1 library, a blastp alignment was used to compare each protein sequence against all other library members (parameters: “-outfmt 6 -evalue 100 -max_hsps 1 -soft_masking false-word_size 7-max_target_seqs 100000”).

Phage ImmunoPrecipitation Sequencing (PhIP-Seq) analyses. PhIP-Seq was performed according to a previously published protocol. (45) Briefly, 0.2 μl of each plasma was individually mixed with the 90-aa human phage library and immunoprecipitated using protein A and protein G coated magnetic beads. A set of 6-8 mock immunoprecipitations (no plasma input) were run on each 96 well plate. Magnetic beads were resuspended in PCR master mix and subjected to thermocycling. A second PCR reaction was employed for sample barcoding. Amplicons were pooled and sequenced on an Illumina NextSeq 500 instrument. PhIP-Seq with the human library was used to characterize autoantibodies in a collection of plasma from healthy donors. For fair comparison to the severe COVID-19 cohort, we first determined the minimum sequencing depth that would have been required to detect the IFN-λ3 reactivity in both of the positive individuals. The present inventors then only considered the 423 data sets from the healthy cohort with sequencing depth greater than this minimum threshold. None of these 423 individuals were found to be reactive to any peptide from IFN-λ3.

Type I/III interferon neutralization assay. IFN-α2 (catalog no. 11100-1), IFN-λ1 (catalog no. 1598-IL-025), and IFN-λ3 (catalog no. 5259-IL-025) were purchased from R&D Systems. 20 μL of patients' crude sera were incubated for 1 hour at room temperature with either 100 U/mL IFN-α2 or 1 ng/mL IFN-λ3, and complete DMEM solvent in a total volume of 200 μL before addition into 7.5×10⁴ A549 cells. After 4-hour incubation, the cells were washed with 1×PBS and cellular mRNA was extracted and purified using RNeasy Plus Mini Kit (Qiagen). 600 ng of extracted mRNA was reverse transcribed using the SuperScript III First-Strand Synthesis System (Life Technologies) and were diluted 10-fold for qPCR runs. The two-step cycling protocol was run on QuantStudio 6 Flex System (Applied Biosystems) and consists of a cycle of 95° C. for 3 minutes, followed by 45 cycles of the following: 95° C. for 15 seconds and 60° C. for 30 seconds. MX1 expression was chosen as a measure of cell stimulation by the interferons, and the relative mRNA expression was normalized by GAPDH expression. The qPCR primer GAPDH and MX1 were obtained from Integrated DNA Technologies (Table 1).

TABLE 1
Primer Sequences

| Names | Sequences |
|---|---|
| T7-Pep2_PCR1_F | 5′-ATAAAGGTGAGGGTAATGTC-3′ (SEQ ID NO: 1) |
| Nextera Index 1 Read | 5′-CAAGCAGAAGACGGCATACGAGAT[17]GTCTC |
| | GTGGGCTCGG-3′ (SEQ ID NO: 2) |
| PhIP_PCR2_F | 5′-AATGATACGGCGACCACCGAGATCTACAC[15]- |
| | GGAGCTGTCGTATTCCAGTC-3′ (SEQ ID NO: 3) |
| P7.2 | 5′-CAAGCAGAAGACGGCATACGA-3′ (SEQ ID NO: 4) |
| T7-Pep2_PCR1_R + ad_min | 5′-CTGGAGTTCAGACGTGTGCTCTTCCGATCAGTTAC |
| | TCGAGCTTATCGT-3′ (SEQ ID NO: 5) |
| Ad min BCX P7 | 5′-CAAGCAGAAGACGGCATACGAGAT[17]CTGGAGT |
| | TCAGACGT-3′ (SEQ ID NO: 6) |
| T7-Pep2.2_SP subA | 5′-CTCGGGGATCCAGGAATTCCGCTGCGT-3′ |
| | (SEQ ID NO: 7) |
| MISEQ_PLATO_R2 | 5′-ATGACGACAAGCCATGGTCGAATCAAACAAGTT |
| | TGTACAAAAAAGTTGGC-3′ (SEQ ID NO: 8) |
| Plato2_15_NextSeq_SP | 5′-GGATCCCCGAGACTGGAATACGACAGCTCC-3′ |
| | (SEQ ID NO: 9) |
| Standard_i7_SP | 5′-GATCGGAAGAGCACACGTCTGAACTCCAGTCAC-3′ |
| | (SEQ ID NO: 10) |
| HL-32_ad | HL-GACGTGTGCTCTTCCGATCAAATTATTTCTAGGT |
| | ACTCGAGCTTATCG (SEQ ID NO: 11) |
| MX1_Forward | 5′-ACCACAGAGGCTCTCAGCAT-3′ (SEQ ID NO: 12) |
| MX1_Reverse | 5′-CTCAGCTGGTCCTGGATCTC-3′ (SEQ ID NO: 13) |
| GAPDH_Forward | 5′-GAGTCAACGGATTTGGTCGT-3′ (SEQ ID NO: 14) |
| GAPDH_Reverse | 5′-TTGATTTTGGAGGGATCTCG-3′ (SEQ ID NO: 15) |
| BT2_F | 5′-GTCAGAGTGACACACTGT-3′ (SEQ ID NO: 16) |
| BT2_R | 5′-AGAGTGACAGTCACAGTG-3′ (SEQ ID NO: 17) |
| BG4_F | 5′-CACTGACTGTGTGAGTGT-3′ (SEQ ID NO: 18) |
| BG4_R | 5′-TGAGACACAGTGAGTCAC-3′ (SEQ ID NO: 19) |
| NT5C1A_F | 5′-CTCACAGACAGACGTCA-3′ (SEQ ID NO: 20) |
| NT5C1A_R | 5′-TGTCAGTCAGTGAGTGTG-3′ (SEQ ID NO: 21) |

### Results

Development of the MIPSA system. The MIPSA Gateway destination vector contains the following key elements: a T7 RNA polymerase transcriptional start site, an isothermal unique clonal identifier (“UCI” barcode) flanked by constant primer binding sequences, a ribosome binding site (RBS), an N-terminal HaloTag fusion protein (891 nt), recombination sequences for ORF insertion, a stop codon, and a homing endonuclease site for plasmid linearization. A recombined ORF-containing pDEST-MIPSA plasmid is shown in FIG. 1A.

The present inventors first sought to establish a library of pDEST-MIPSA plasmids containing stochastic, isothermal UCIs located between the transcriptional start site and the ribosome binding site. A degenerate oligonucleotide pool was synthesized, comprising melting temperature (Tm) balanced sequences: (SW)₁₈-AGGGA-(SW)₁₈, where S represents an equal mix of C and G, while W represents an equal mix of A and T (FIG. 1B). The present inventors reasoned that this inexpensive pool of sequences would (i) provide sufficient complexity (2³⁶˜7×10¹⁰) for unique ORF labeling, (ii) amplify without distortion, and (iii) serve as ORF-specific forward and reverse qPCR primer binding sites for measurement of individual UCIs of interest. The degenerate oligonucleotide pool was amplified by PCR, restriction cloned into the MIPSA destination vector and transformed into E. coli (Methods). About 800,000 transformants were scraped off selection plates to obtain the pDEST-MIPSA UCI plasmid library. ORFs encoding the housekeeping gene glyceraldehyde-3-phosphate dehydrogenase (GAPDH) and a known autoantigen, tripartite motif containing-21 (TRIM21, commonly known as Ro52), were separately recombined into the pDEST-MIPSA UCI plasmid library and used in the following experiments. Single barcoded GAPDH and TRIM21 clones were isolated and sequenced.

The MIPSA procedure involves reverse transcription of the stochastic barcode using a succinimidyl ester (O2)-haloalkane (HaloLigand)-conjugated reverse transcription (RT) primer. The bound RT primer should not interfere with the assembly of the E. coli ribosome and initiation of translation, but should be sufficiently proximal such that coupling of the HaloLigand-HaloTag-protein complex might hinder additional rounds of translation. The present inventors tested a series of RT primers that anneal at distances ranging from −30 nucleotides to +7 nucleotides (5′ to 3′) from the 3′ end of the RBS (FIG. 1D). Based on the yield of protein product from mRNA saturated with primers at these varying locations, the present inventors selected the −20 position as it did not interfere with translation efficiency (FIG. 1E). In contrast, RT from primers located within 20 nucleotides of the RBS diminished or abolished protein translation. This result agrees with the estimated footprint of assembled 70S E. coli ribosomes, which have been shown to protect a minimum of 15 nucleotides of mRNA.(13)

The present inventors next assessed the ability of SuperScript IV to perform reverse transcription from a primer labeled with the HaloLigand at its 5′ end, and the ability of the HaloTag-TRIM21 protein to form a covalent bond with the HaloLigand-conjugated primer during the translation reaction. HaloLigand conjugation and purification followed Gu et al. (Materials and Methods, FIG. 6). (14) Either unconjugated RT primer or aHaloLigand-conjugated RT primer was used for RT of the barcoded HaloTag-TRIM21 mRNA. The translation product was then immunoprecipitated (IPed) with serum from a healthy donor or serum from a TRIM21 (Ro52) autoantibody-positive patient with Sjögren's Syndrome (SS). The SS serum efficiently IPed the TRIM21 protein, regardless of RT primer conjugation, but only pulled down the TRIM21 cDNA UCI when the HaloLigand-conjugated primer was used in the RT reaction (FIG. 1F-G).

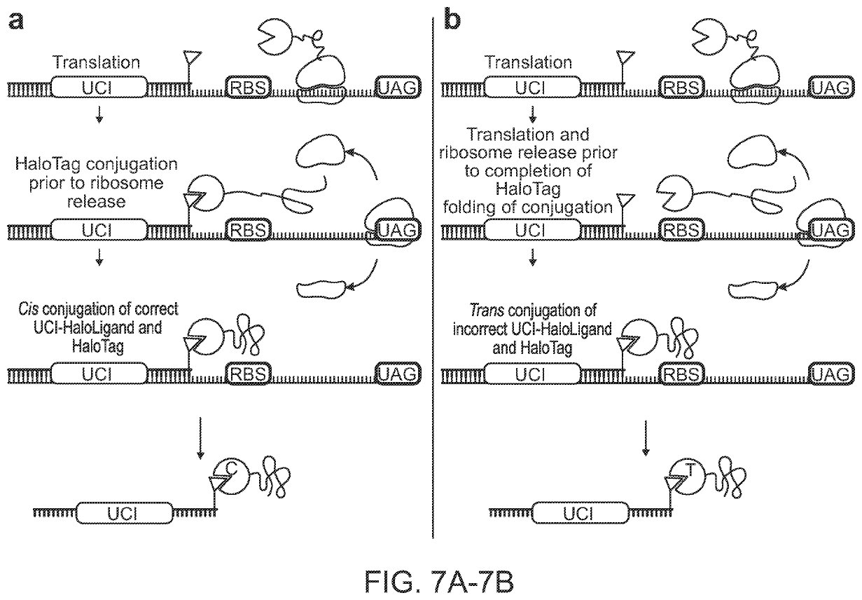

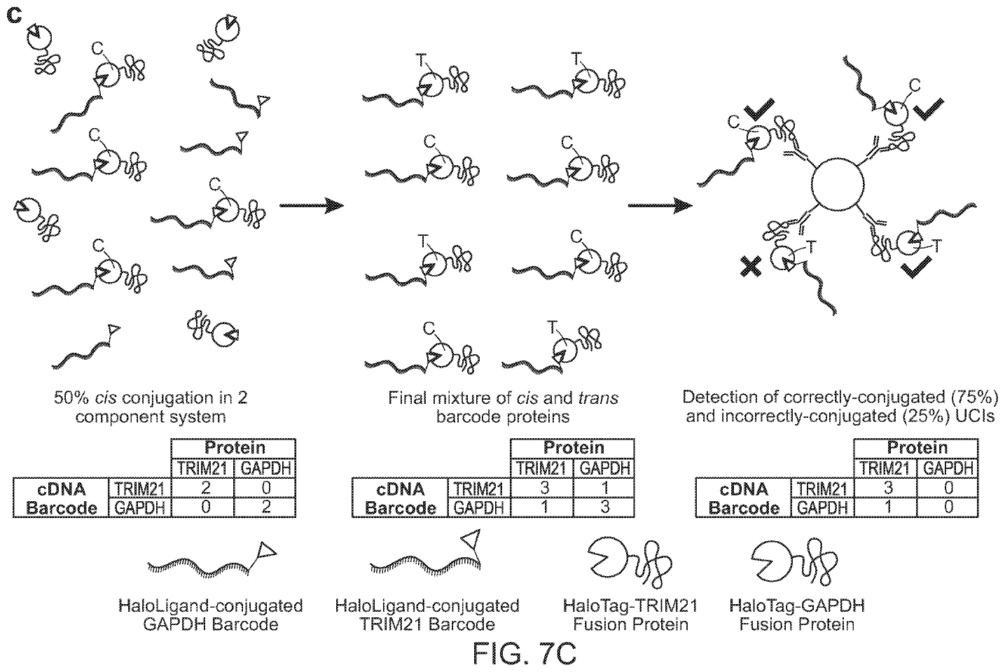

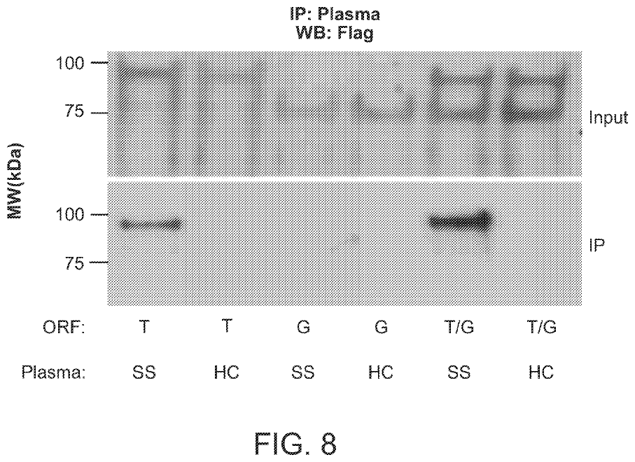

Assessing cis versus trans UCI barcoding. While the previous experiment indicated that indeed the HaloLigand does not impede RT priming, and that the HaloTag can form a covalent bond with the HaloLigand during the translation reaction, it did not elucidated the amount of cis (intra-complex) and trans (inter-complex) HaloTag-UCI conjugation (FIG. 7). In order to measure the amount of cis and trans HaloTag-UCI-conjugation. GAPDH and TRIM21 mRNAs were separately reverse transcribed (using HaloLigand primer) and then either mixed 1:1 or kept separate for in vitro translation. As expected, translation of the mixture produced roughly equivalent amounts of each protein compared to the individual translations (FIG. 8). SS serum specifically IPed TRIM21 protein regardless of translation condition (FIG. 8, IPed fraction). However, the present inventors noted that while the SS IPs contained high levels of the TRIM21 UCI, as intended, more of the GAPDH UCI was pulled down by the SS serum compared to that by the HC serum when the mRNA was mixed mixed prior to translation. This indicates that indeed some trans barcoding occurs (FIG. 2A). The present inventors estimate that ˜50% of the protein is cis-barcoded, with the remaining 50% trans barcoded protein equally represented by both proteins. Thus, in this two-compartment system, 25% of the TRIM21 protein is conjugated to the GAPDH-UCI.

In the setting of a complex library, even if ˜50% of the protein is trans barcoded, this unwanted side product would be uniformly distributed across all members of the library. The present inventors tested this using a model MIPSA library composed of 100-fold excess of a second GAPDH clone, which was combined with a 1:1 mixture of the first GAPDH and TRIM21 clones (FIG. 2B). The present inventors additionally developed a sequencing workflow utilizing a PCR spike-in sequence for absolute quantification of each UCI. IP with SS serum using the optimized protocol resulted in specific IP of the TRIM21-UCI, with negligible trans-coupled GAPDH-UCI IP detected (FIG. 2B). Using the spiked-in sequence for absolute quantification, and assuming pull down of 100% of the TRIM21 protein, the present inventors calculated a cis coupling efficiency of about 0.2% (i.e., 0.2% of input TRIM21 RNA molecules were converted into the intended UCI-coupled TRIM21 proteins.

Establishing and deconvoluting a stochastically barcoded human ORFeome MIPSA library. The sequence-verified human ORFeome v8.1 is composed of 12,680 clonal ORFs mapping to 11,437 genes in pDONR223. (15) Five subpools of the library were created, each composed of roughly ˜2,500 similarly sized ORFs. Each of the five subpools was separately recombined into the pDEST-MIPSA UCI plasmid library and transformed to obtain ˜10-fold ORF coverage (˜30,000 clones per subpool). Each subpool was assessed via Bioanalyzer electrophoresis, sequencing of ˜20 colonies, and Illumina sequencing of the superpool. The TRIM21 plasmid was spiked into the superpooled hORFeome library at 1:10,000-comparable to a typical library member. The SS IP experiment was then performed on the hORFeome MIPSA library, using sequencing as a readout. The reads from all barcodes in the library, including the spiked-in TRIM21, are shown in FIG. 2C. The SS autoantibody-dependent enrichment of TRIM21 (17-fold) was similar to the simple system (FIG. 2D). Assuming the coupling efficiencies derived earlier, the present inventors estimate that about 6×10⁵ molecules of correctly cis-coupled TRIM21 molecules (and thus each library member on average) was input to the IP reaction.

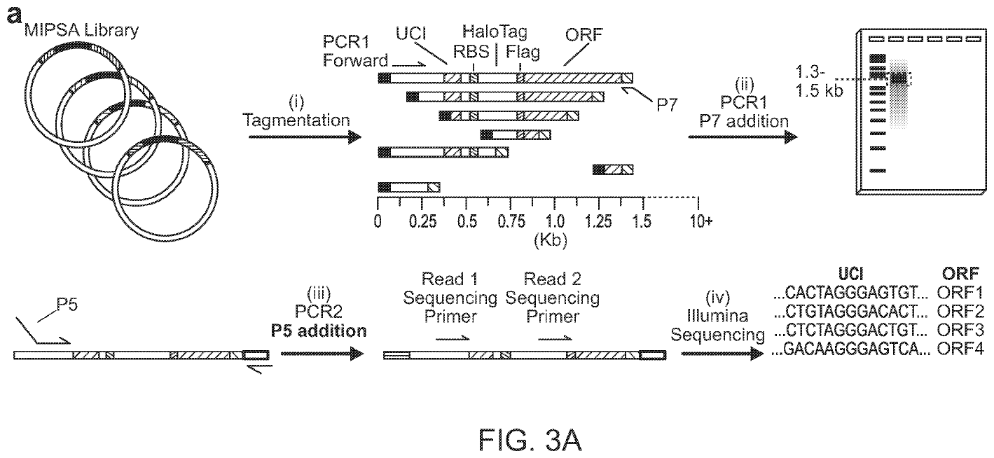

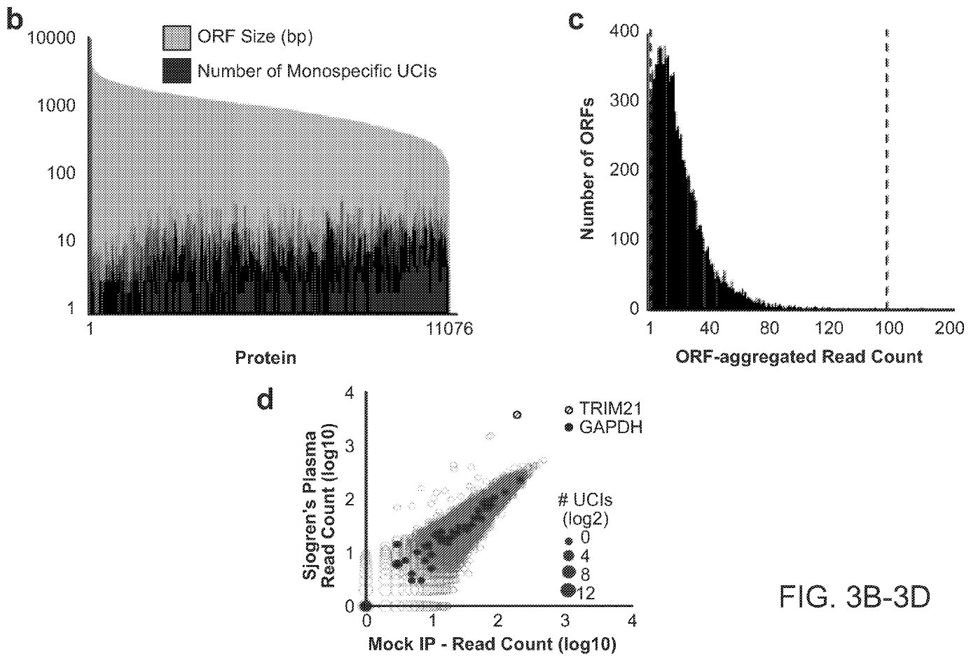

Next, the present inventors established a system for creating a UCI-ORF lookup dictionary, using tagmentation and sequencing (FIG. 3A). Sequencing the 5′ 50 nt of the ORF inserts detected 11,300 of the 11,887 unique 5′ 50 nt sequences. Of the 153,161 unique barcodes detected, 82.9% (126,975) were found to be associated with a single ORF. Each ORF was uniquely associated with a median of 9 UCIs, ranging from 0 to 123 UCIs (FIG. 3B). Aggregating the reads corresponding to each ORF, over 99% of the represented ORFs were present within a 10-fold difference of the median ORF abundance (FIG. 3C). Taken together, these data indicated that the present inventors established a uniform library of 11,300 stochastically indexed human ORFs, and sufficiently defined a dictionary for downstream analyses. FIG. 3D shows the scatterplot of FIG. 2C but with the 47 dictionary-decoded GAPDH UCIs (corresponding to two GAPDH isoforms present in the hORFeome library) appearing along the y=x diagonal as expected.

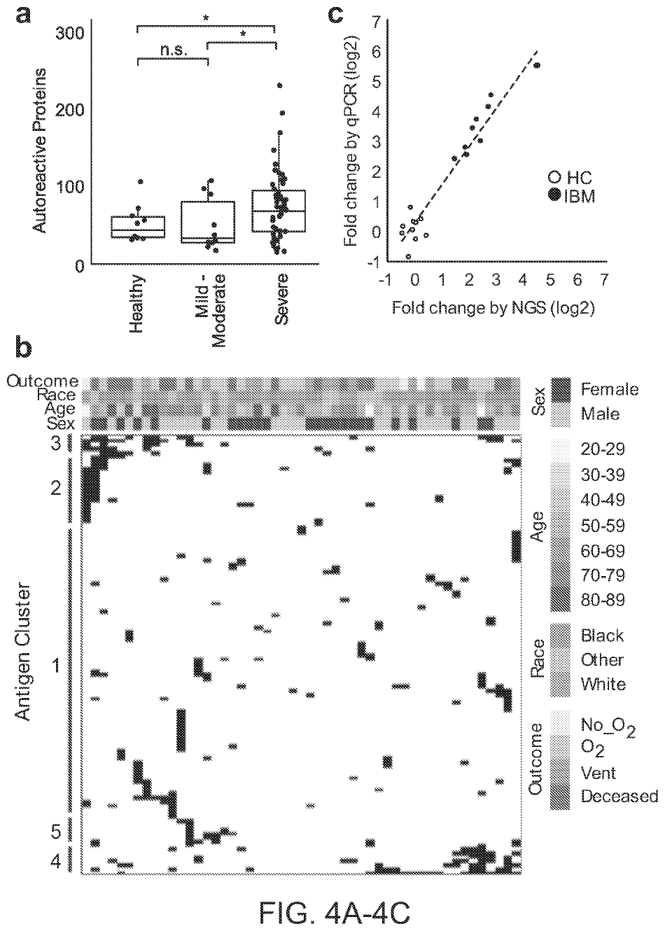

Unbiased MIPSA analysis of autoantibodies associated with severe COVID-19. Several recent reports have described elevated autoantibody reactivities in patients with severe COVID-19. (16-20) The present inventors therefore used MIPSA with the human ORFeome library for unbiased identification of autoreactivities in the plasma of 55 severe COVID-19 patients. For comparison, the present inventors used MIPSA to detect autoreactivities in plasma from 10 healthy donors and 10 COVID-19 convalescent plasma donors who had not been hospitalized (Table 2). Each sample was compared to a set of 8 “mock IPs”, which contained all reaction components except for serum. This comparison to mock IPs accounts for bias in the library and background binding. Importantly, the informatic pipeline used to detect antibody-dependent reactivity yielded a median of 5 false positive UCI hits per mock IP (ranging from 2 to 9). IPs using serum from severe COVID-19 patients, however, yielded a mean of 132 reactive UCIs, significantly more than the mean of 93 reactive UCIs among the controls (p=0.018, t-test). Collapsing UCIs to their corresponding proteins yielded a mean of 83 reactive proteins among severe COVID-19 patients, which was significantly more than the mean of 63 reactive proteins among controls (FIG. 4A, p=0.019, t-test).

TABLE 2
Study Population
Study Population

| Group | | # | Age | Sex | Black | White | Other |
|---|---|---|---|---|---|---|---|
| Severe | Died | 17 | 67 (27, 87) | F: 8, M: 9 | 9 | 4 | 4 |
| COVID-19 | Ventilated | 13 | 67 (27, 82) | F: 9, M: 4 | 4 | 4 | 5 |
| | Got O2 | 22 | 52 (27, 82) | F: 9, M: 13 | 8 | 8 | 6 |
| | No O2 | 3 | 46 (22, 49) | F: 0, M: 3 | 0 | 3 | 0 |
| COVID-19 | Mild/Mod | 10 | 35 (19, 55) | F: 6, M: 4 | 0 | 8 | 2 |
| Controls | Healthy Control | 10 | 41.5 (22, 66) | F: 3, M: 7 | 3 | 5 | 2 |
| Myositis | Inclusion Body | 10 | 53.9 (43.6, 60.6) | F: 7, M: 3 | 1 | 7 | 2 |
| | Healthy Control | 10 | 36.5 (20, 60) | F: 5, M: 5 | 2 | 8 | 0 |

The present inventors next examined proteins in the severe COVID-19 IPs that had at least two reactive UCIs, which were reactive in at least one severe patient, and which were not reactive in more than one control (healthy or mild/moderate convalescent plasma). Proteins were excluded if they were reactive in a single severe patient and single control. The 115 proteins that met these criteria are shown in the clustered heatmap of FIG. 4B. Fifty two of the 55 severe COVID-19 patients exhibited reactivity to at least one of these proteins. The present inventors noted co-occurring protein reactivities in multiple individuals, the vast majority of which lack homology by protein sequence alignment.

One notable autoreactivity cluster (FIG. 4B) includes the 5′-nucleotidase, cytosolic 1A (NT5C1A), which is highly expressed in skeletal muscle and is the most well-characterized autoantibody target in inclusion body myositis (IBM). Multiple UCIs linked to NT5C1A were significantly increased in 3 of the 55 severe COVID-19 patients (5.5%). NT5C1A autoantibodies have been reported in up to 70% of IBM patients, (1) in ˜20% of SS patients and in up to ˜5% of healthy donors. (21) The frequency of NT5C1A reactivity in the severe COVID-19 cohort is there not necessarily elevated. However, the present inventors wondered whether MIPSA would be able to reliably distinguish between healthy donor and IBM plasma based on NT5C1A reactivity. The present inventors tested plasma from 10 healthy donors and 10 IBM patients, the latter of whom were selected based on NT5C1A seropositivity as determined by PhIP-Seq. (1) The clear separation of patients from controls in this independent cohort suggests that MIPSA may indeed have utility in clinical diagnostic testing using either qPCR or sequencing, which were tightly correlated readouts (FIG. 4C).

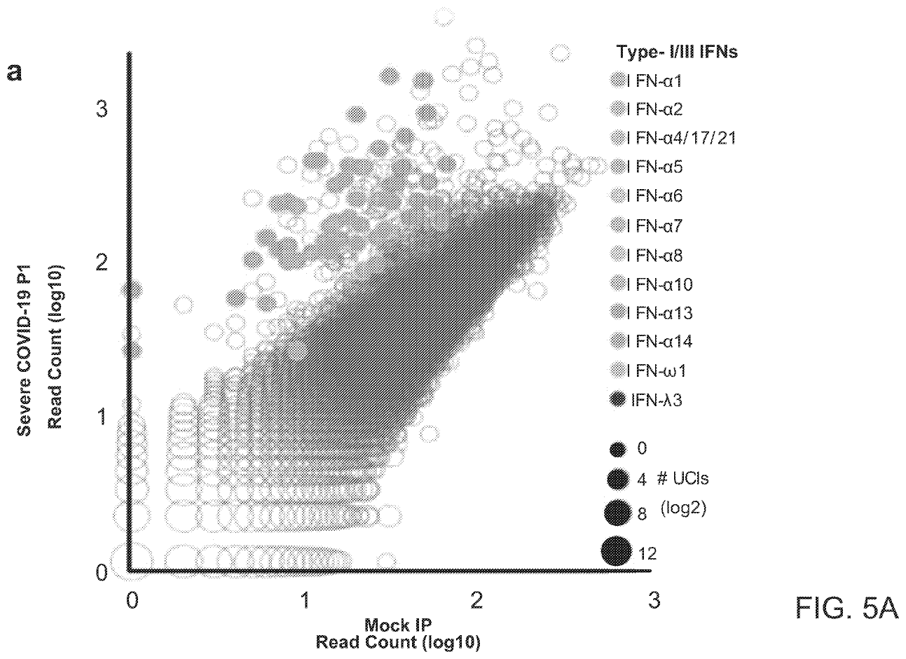

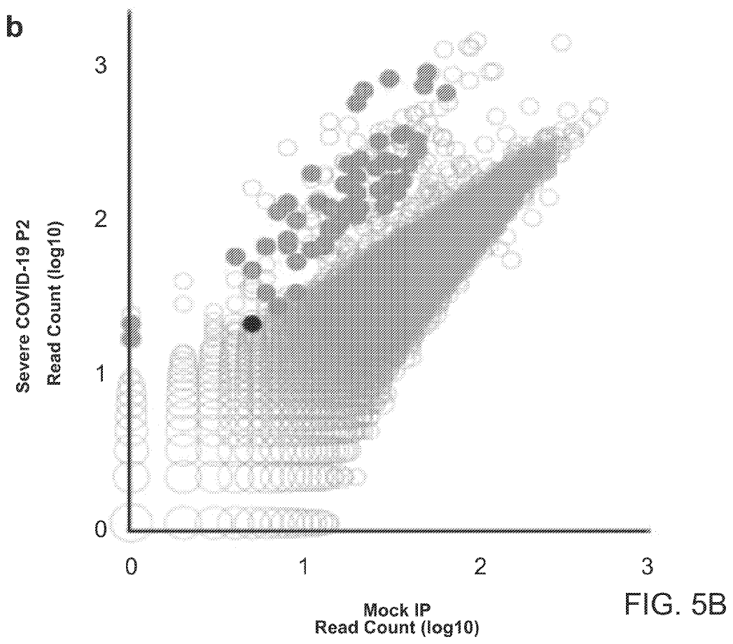

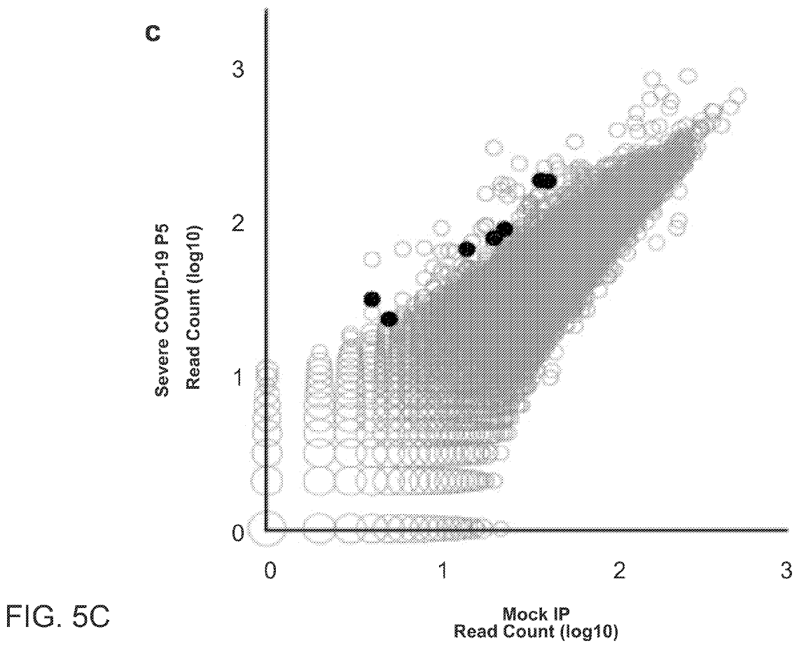

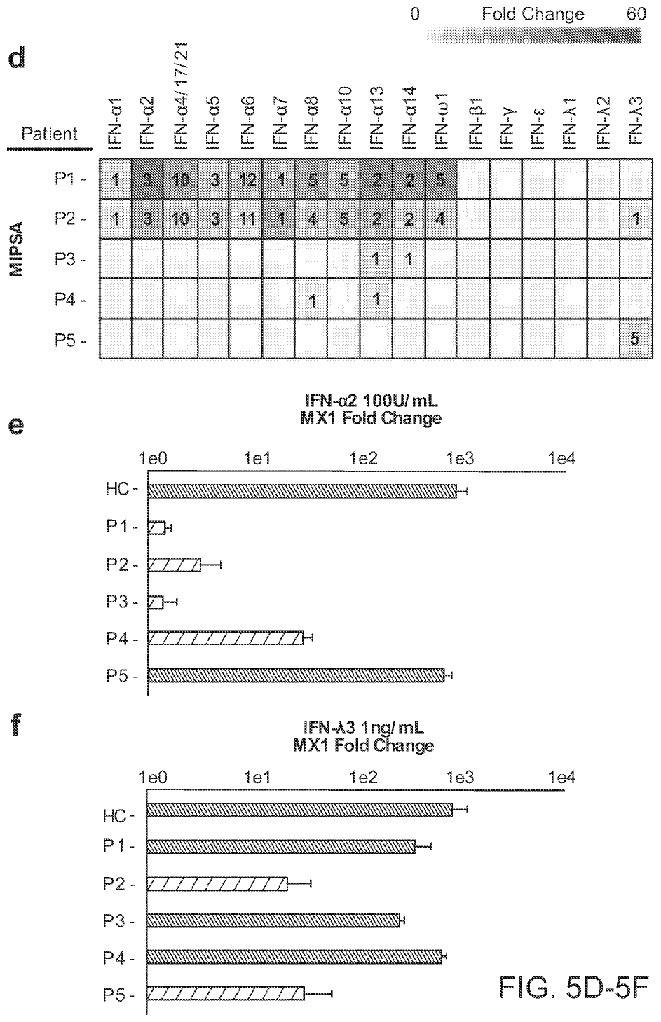

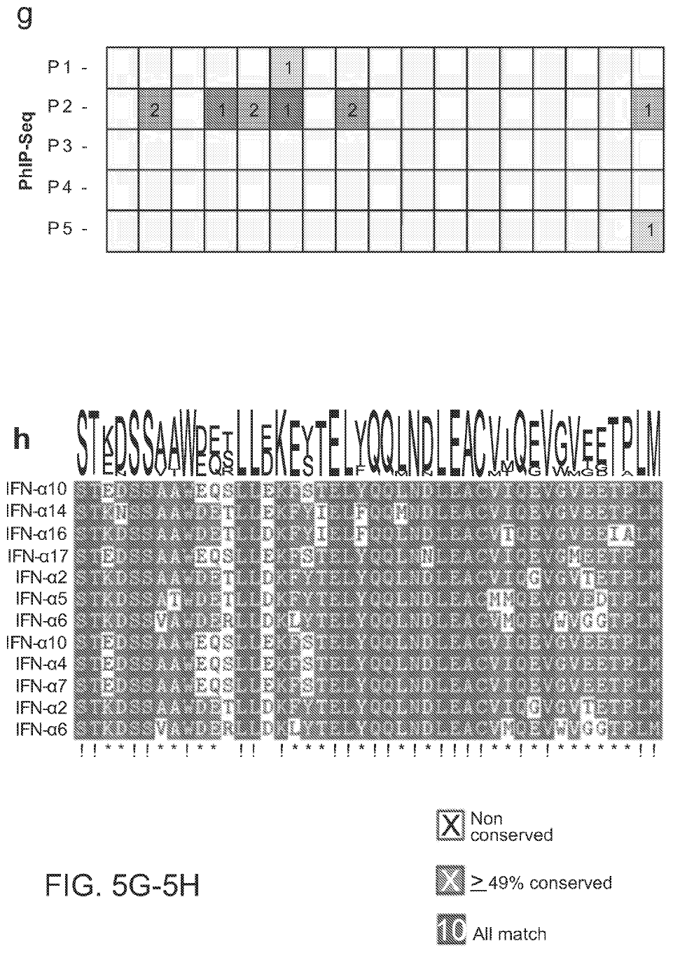

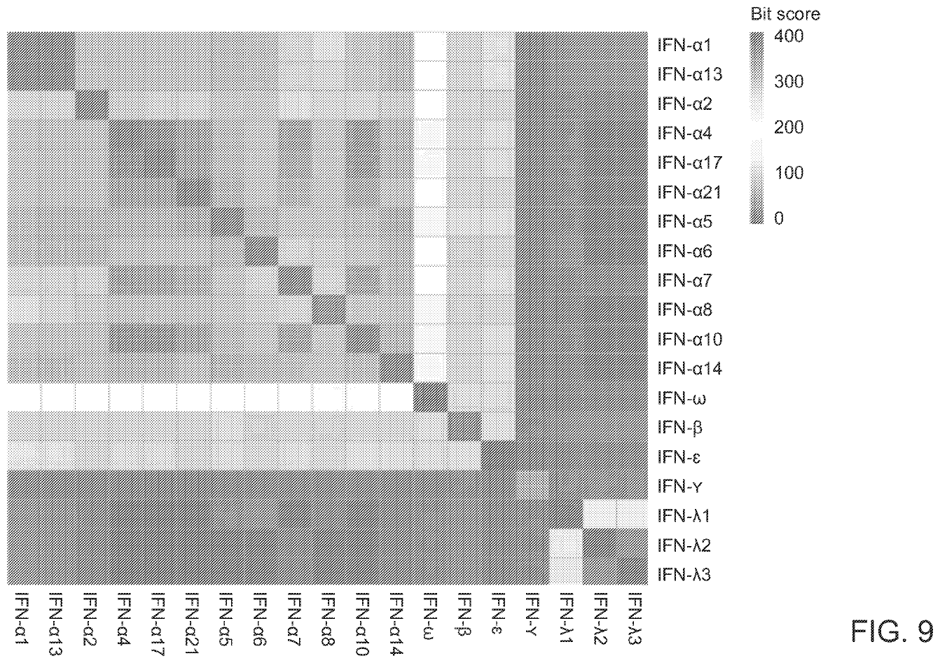

Type I and III interferon-neutralizing autoantibodies in severe COVID-19. Neutralizing autoantibodies targeting type I interferons alpha (IFN-α) and omega (IFN-ω) have been associated with severe COVID-19.(17, 22, 23) All type I interferons except IFN-α16 are represented in the human MIPSA library and dictionary. However, IFN-α4, IFN-α17, and IFN-α21 are indistinguishable by sequencing the first 50 nucleotides of their encoding ORF sequences. Two of the severe COVID-19 patients in this cohort (3.6%) exhibited dramatic IFN-α autoreactivity (43 and 41 UCIs, across 10 distinct IFN-α ORFs, along with 5 and 2 IFN-ω UCIs, FIG. 5A-5B). The extensive co-reactivity of these proteins is likely attributable to their sequence homology (FIG. 9). By requiring at least 2 IFN UCIs to be considered positive, we identified three additional severe COVID-19 patients with lower levels of reactivity, each with only 2 reactive IFN-α UCIs. Interestingly, one plasma (P5) precipitated 5 UCIs from the type III interferon IFN-λ3, but no UCI from any type I or II interferon (FIG. 5C-5D). None of the healthy or non-hospitalized COVID-19 controls were positive for 2 or more interferon UCIs.

Incubation of A549 human adenocarcinomatous lung epithelial cells with 100 U/ml IFN-α2 or 1 ng/ml of IFN-λ3 for 4 hours in serum-free medium resulted in a robust upregulation of the IFN-response gene MX1, by ˜1,000-fold and ˜100-fold, respectively. Pre-incubation of the IFN-α2 with P1, P2 or P3's plasma completely abolished the A549 interferon response (FIG. 5E). The plasma with the weakest IFN-α reactivity by MIPSA (P4) partially neutralized the cytokine. Neither HC nor P5 plasma had any effect on the response of A549 cells to IFN-α. However, pre-incubation of the IFN-λ3 with the MIPSA-reactive plasmas, P2 and P5, neutralized the cytokine (FIG. 5F). None of the other plasma (HC, P1, P3 or P4) had any effect on the response of A549 cells to IFN-λ. In summary, antibody profiling of this severe COVID-19 cohort identified strongly neutralizing IFN-α autoantibodies in 5.5% of patients and strongly neutralizing IFN-λ3 autoantibodies in 3.6% of patients, with a single patient (1.8%) harboring both autoreactivites.

The present inventors wondered if PhIP-Seq with a 90-aa human peptidome library (24) might also detect interferon antibodies in this cohort. PhIP-Seq detected IFN-α reactivity in plasma from P1 and P2, although to a much lesser extent (FIG. 5G). The two weaker IFN-α reactivities detected by MIPSA in the plasma of P3 and P4 were both missed by PhIP-Seq. PhIP-Seq identified a single additional weakly IFN-α reactive sample, which was negative by MIPSA (not shown). Detection of type III interferon autoreactivity (directed exclusively at IFN-λ3) agreed perfectly between the two technologies. PhIP-Seq data was used to narrow the location of a dominant epitope in the type I and type III autoantigens (FIG. 5H-5I).

The present inventors next wondered about the prevalence of the IFN-λ3 autoreactivity in the general population, and whether it might be increased among patients with severe COVID-19. PhIP-Seq was used to profile the plasma of 423 healthy controls, none of whom were found to have detectable IFN-λ3 autoreactivity. These data suggest that IFN-λ3 autoreactivity may be more frequent among individuals with severe COVID-19. This is the first report describing neutralizing anti-IFNλ autoantibodies, and therefore proposes a potentially novel pathogenic mechanism contributing to life-threatening COVID-19 in a subset patients.

### Example 2: Neutralizing IFNL3 Autoantibodies in Severe COVID-19 Identified Via Protein Display Technology

Autoantibodies detected in severe COVID-19 patients using MIPSA. The association between autoimmunity and severe COVID-19 disease is increasingly appreciated. In a cohort of 55 hospitalized individuals, the present inventors detected multiple established autoantibodies, including one that the present inventors have previously linked to inclusion body myositis.(1) The present inventors then tested the performance of MIPSA for detecting the NT5C1A autoantibody in a separate cohort of seropositive IBM patients and healthy controls. The results support future efforts in evaluating the clinical utility of MIPSA for standardized, comprehensive autoantibody testing. Such tests could utilize either single-plex qPCR or unbiased sequencing as a readout.

While clusters of autoreactivities were observed in multiple individuals, it is not clear what role, if any, they may play in severe COVID-19. In larger scale studies, the present inventors expect that patterns of co-occurring reactivity, or reactivities towards proteins with related biological functions, may ultimately define new autoimmune syndromes associated with severe COVID-19. Neutralizing IFN-α and IFN-ω autoantibodies have been described in patients with severe COVID-19 and are presumed to be pathogenic.(17) These likely pre-existing autoantibodies, which occur very rarely in the general population, block restriction of viral replication in cell culture, and are thus likely to interfere with disease resolution. This discovery paved the way to identifying a subset of individuals at risk for life-threatening COVID-19 pneumonia, and proposed a potential therapeutic avenue utilizing interferon beta, which is not neutralized by these autoantibodies. In the present study, MIPSA identified two individuals with extensive reactivity to the entire family of IFN-α cytokines. Indeed, plasma from both individuals, plus one individual with weaker IFN-α reactivity detected by MIPSA, robustly neutralized recombinant IFN-α2 in a lung adenocarcinomatous cell culture model. Unexpectedly, one individual in the cohort without IFN-α reactivity pulled down 5 IFN-λ3 UCIs. A second IFN-α autoreactive individual also pulled down a single IFN-λ3 UCI. The same autoreactivities were also detected using PhIP-Seq. Interestingly, neither MIPSA nor PhIP-Seq detected reactivity to IFN-λ2, despite their high degree of sequence homology (FIG. 9). The present inventors tested the IFN-λ3 neutralizing capacity of these patients' plasma, observing near complete ablation of the cellular response to the recombinant cytokine (FIG. 5F). These data propose IFN-λ3 autoreactivity is a new, potentially pathogenic mechanism contributing to severe COVID-19 disease.

Type III IFNs (IFN-λ, also known as IL-28/29) are cytokines with potent anti-viral activities that act primarily at barrier sites. The IFN-λR1/IL-10RB heterodimeric receptor for IFN-λ is expressed on lung epithelial cells and is important for the innate response to viral infection. Mordstein et al., determined that in mice, IFN-λ diminished pathogenicity and suppressed replication of influenza viruses, respiratory syncytial virus, human metapneumovirus, and severe acute respiratory syndrome coronavirus (SARS-CoV-1).(32) It has been proposed that IFN-λ exerts much of its antiviral activity in vivo via stimulatory interactions with immune cells, rather than through induction of the antiviral cell state.(33) Importantly, IFN-λ has been found to robustly restrict SARS-CoV-2 replication in primary human bronchial epithelial cells(34), primary human airway epithelial cultures(35) and primary human intestinal epithelial cells(36). Collectively, these studies suggest multifaceted mechanisms by which neutralizing IFN-λ autoantibodies may exacerbate SARS-CoV-2 infections.

Casanova et al. did not detect any type III IFN neutralizing antibodies among 101 individuals with type I IFN autoantibodies tested.(17) In the present inventors' study, one of the three IFN-α autoreactive individuals (P2, a 22-year-old male) also harbored autoantibodies that neutralized IFN-λ3. It is possible that this co-reactivity is extremely rare and thus not represented in the Casanova cohort. Alternatively, it is possible that the differing assay conditions exhibit different detection sensitivity. Whereas Casanova et al. cultured A549 cells with IFN-λ3 at 50 ng/ml and without plasma preincubation, the present inventors cultured A549 cells with IFN-λ3 at 1 ng/ml after pre-incubation with plasma for one hour. Their readout of STAT3 phosphorylation may also provide different detection sensitivity compared with the upregulation of MX1. A larger study is needed to determine the true frequency of these reactivities in severe COVID-19 patients and matched controls. Here, the present inventors report neutralizing IFN-α and IFN-λ3 autoantibodies in 3 (5.5%) and 2 (3.6%), respectively, of 55 individuals with severe COVID-19. IFN-λ3 autoantibodies were not detected via PhIP-Seq in a larger cohort of 541 healthy controls collected prior to the pandemic.

Type III interferons have been proposed as a therapeutic modality for SARS-CoV-2 infection,(35, 37-41) and there are currently three ongoing clinical trials to test pegylated IFN-λ1 for efficacy in reducing morbidity and mortality associated with COVID-19 (ClinicalTrials.gov Identifiers: NCT04343976, NCT04534673, NCT04344600). One recently completed double-blind, placebo-controlled trial, NCT04354259, reported a significant reduction by 2.42 log copies per mL of SARS-CoV-2 at day 7 among mild to moderate COVID-19 patients in the outpatient setting (p=0.0041). (42) Future studies will determine whether anti-IFN-λ3 autoantibodies are pre-existing or arise in response to SARS-CoV-2 infection, and how often they also cross-neutralize IFN-λ1. Based on sequence alignment of IFN-λ1 and IFN-λ3 (˜29% homology, FIG. 9), however, cross-neutralization is expected to be rare, raising the possibility that patients with neutralizing IFN-λ3 autoantibodies may especially derive benefit from pegylated IFN-λ1 treatment.

### Conclusion

MIPSA is a new self-assembling protein display technology with key advantages over alternative approaches. It has properties that complement techniques like PhIP-Seq, and MIPSA libraries can be conveniently screened in the same reactions with programmable phage display libraries. The MIPSA protocol presented here requires cap-independent cell free translation, but future adaptations may overcome this limitation. Applications for MIPSA-based studies include protein-protein, protein-antibody, and protein-small molecule interaction studies, and include unbiased analyses of post-translational modifications. Here, the present inventors used MIPSA to discover neutralizing IFN-λ3 autoantibodies, among many other potentially pathogenic autoreactivities, which may contribute to life-threatening COVID-19 pneumonia in a subset of at-risk individuals.

### REFERENCES

1. H. B. Larman et al., Cytosolic 5′-nucleotidase 1A autoimmunity in sporadic inclusion body myositis. Annals of neurology 73, 408-418 (2013).
2. G. J. Xu et al., Viral immunology. Comprehensive serological profiling of human populations using a synthetic human virome. Science 348, aaa0698 (2015).
3. E. Shrock et al., Viral epitope profiling of COVID-19 patients reveals cross-reactivity and correlates of severity. Science 370, (2020).
4. D. R. Monaco et al., Profiling serum antibodies with a pan allergen phage library identifies key wheat allergy epitopes. Nat Commun 12, 379 (2021).
5. S. F. Kingsmore, Multiplexed protein measurement: technologies and applications of protein and antibody arrays. Nat Rev Drug Discov 5, 310-320 (2006).
6. T. Kodadek, Protein microarrays: prospects and problems. Chem Biol 8, 105-115 (2001).
7 N. Ramachandran, E. Hainsworth, G. Demirkan, J. LaBaer, On-chip protein synthesis for making microarrays. Methods Mol Biol 328, 1-14 (2006).
8. S. Rungpragayphan, T. Yamane, H. Nakano, SIMPLEX: single-molecule PCR-linked in vitro expression: a novel method for high-throughput construction and screening of protein libraries. Methods Mol Biol 375, 79-94 (2007).
9. J. Zhu et al., Protein interaction discovery using parallel analysis of translated ORF
10. G. Liszczak, T. W. Muir, Nucleic Acid-Barcoding Technologies: Converting DNA Sequencing into a Broad-Spectrum Molecular Counter. Angew Chem Int Ed Engl 58, 4144-4162 (2019).
11. G. V. Los et al., HaloTag: a novel protein labeling technology for cell imaging and protein analysis. ACS Chem Biol 3, 373-382 (2008).
12. J. Yazaki et al., HaloTag-based conjugation of proteins to barcoding-oligonucleotides. Nucleic Acids Res 48, e8 (2020).
13. F. Mohammad, R. Green, A. R. Buskirk, A systematically-revised ribosome profiling method for bacteria reveals pauses at single-codon resolution. Elife 8, (2019).
14. L. Gu et al., Multiplex single-molecule interaction profiling of DNA-barcoded proteins. Nature 515, 554-557 (2014).
15. X. Yang et al., A public genome-scale lentiviral expression library of human ORFs. Nat Methods 8, 659-661 (2011).
16. C. R. Consiglio et al., The Immunology of Multisystem Inflammatory Syndrome in Children with COVID-19. Cell 183, 968-981 e967 (2020).
17. P. Bastard et al., Autoantibodies against type I IFNs in patients with life-threatening COVID-19. Science 370, (2020).
18. Y. Zuo et al., Prothrombotic autoantibodies in serum from patients hospitalized with COVID-19. Sci Transl Med 12, (2020).
19. L. Casciola-Rosen et al., IgM autoantibodies recognizing ACE2 are associated with severe COVID-19. medRxiv, (2020).
20. M. C. Woodruff, R. P. Ramonell, F. E. Lee, I. Sanz, Broadly-targeted autoreactivity is common in severe SARS-CoV-2 Infection. medRxiv, (2020).
21. T. E. Lloyd et al., Cytosolic 5′-Nucleotidase 1A As a Target of Circulating Autoantibodies in Autoimmune Diseases. Arthritis Care Res (Hoboken) 68, 66-71 (2016).
22. E. Y. Wang et al., Diverse Functional Autoantibodies in Patients with COVID-19. medRxiv, (2020).
23. S. Gupta, S. Nakabo, J. Chu, S. Hasni, M. J. Kaplan, Association between anti-interferon-alpha autoantibodies and COVID-19 in systemic lupus erythematosus. medRxiv, (2020).
24. G. J. Xu et al., Systematic autoantigen analysis identifies a distinct subtype of scleroderma with coincident cancer. Proc Natl Acad Sci USA, (2016).
25. M. Stoeckius et al., Simultaneous epitope and transcriptome measurement in single cells. Nat Methods 14, 865-868 (2017).
26. I. Setliff et al., High-Throughput Mapping of B Cell Receptor Sequences to Antigen Specificity. Cell 179, 1636-1646 e1615 (2019).
27. S. K. Saka et al., Immuno-SABER enables highly multiplexed and amplified protein.
28. M. A. Jongsma, R. H. Litjens, Self-assembling protein arrays on DNA chips by auto-labeling fusion proteins with a single DNA address. Proteomics 6, 2650-2655 (2006).
29. A. Gautier et al., An engineered protein tag for multiprotein labeling in living cells. Chem Biol 15, 128-136 (2008).
30. A. J. Samelson et al., Kinetic and structural comparison of a protein's cotranslational folding and refolding pathways. Sci Adv 4, eaas9098 (2018).
31. L. Tosi et al., Long-adapter single-strand oligonucleotide probes for the massively multiplexed cloning of kilobase genome regions. Nat Biomed Eng 1, (2017).
32. M. Mordstein et al., Lambda interferon renders epithelial cells of the respiratory and gastrointestinal tracts resistant to viral infections. J Virol 84, 5670-5677 (2010).
33. N. Ank et al., Lambda interferon (IFN-lambda), a type III IFN, is induced by viruses and IFNs and displays potent antiviral activity against select virus infections in vivo. J Virol 80, 4501-4509 (2006).
34. I. Busnadiego et al., Antiviral Activity of Type I, II, and III Interferons Counterbalances ACE2 Inducibility and Restricts SARS-CoV-2. mBio 11, (2020).
35. A. Vanderheiden et al., Type I and Type III Interferons Restrict SARS-CoV-2 Infection of Human Airway Epithelial Cultures. J Virol 94, (2020).
36. M. L. Stanifer et al., Critical Role of Type III Interferon in Controlling SARS-CoV-2 Infection in Human Intestinal Epithelial Cells. Cell Rep 32, 107863 (2020).
37. I. E. Galani et al., Untuned antiviral immunity in COVID-19 revealed by temporal type I/III interferon patterns and flu comparison. Nat Immunol 22, 32-40 (2021).
38. U. Felgenhauer et al., Inhibition of SARS-CoV-2 by type I and type III interferons. J Biol Chem 295, 13958-13964 (2020).
39. T. R. O'Brien et al., Weak Induction of Interferon Expression by Severe Acute Respiratory Syndrome Coronavirus 2 Supports Clinical Trials of Interferon-lambda to Treat Early Coronavirus Disease 2019. Clin Infect Dis 71, 1410-1412 (2020).
40. E. Andreakos, S. Tsiodras, COVID-19: lambda interferon against viral load and hyperinflammation. EMBO Mol Med 12, e12465 (2020).
41. L. Prokunina-Olsson et al., COVID-19 and emerging viral infections: The case for interferon lambda. J Exp Med 217, (2020).
42. J. J. Feld et al., Peginterferon lambda for the treatment of outpatients with COVID-19: a phase 2, placebo-controlled randomised trial. Lancet Respir Med, (2021).
43. D. Mohan et al., Publisher Correction: PhIP-Seq characterization of serum antibodies using oligonucleotide-encoded peptidomes. Nature protocols 14, 2596 (2019).
44. C. Tuckey, H. Asahara, Y. Zhou, S. Chong, Protein synthesis using a reconstituted cell-free system. Curr Protoc Mol Biol 108, 16 31 11-22 (2014).
45. D. Mohan et al., PhIP-Seq characterization of serum antibodies using oligonucleotide-encoded peptidomes. Nature protocols 13, 1958-1978 (2018).
46. S. L. Klein et al., Sex, age, and hospitalization drive antibody responses in a COVID-19 convalescent plasma donor population. J Clin Invest 130, 6141-6150 (2020).
47. R. A. Zyskind I, Zimmerman J, Naiditch H, Glatt A E, Pinter A, Theel E S, Joyner M J, Hill D A, Lieberman M R, Bigajer E, Stok D, Frank E, Silverberg J I, SARS-CoV-2 Seroprevalence and Symptom Onset in Culturally-Linked Orthodox Jewish Communities Across Multiple Regions in the United States. JAMA Open Network In Press, (2021).
48. Correction: Patient Trajectories Among Persons Hospitalized for COVID-19. Ann Intern Med 174, 144 (2021).
49. M. R. Rose, E. I. W. Group, 188th ENMC International Workshop: Inclusion Body Myositis, 2-4 Dec. 2011, Naarden, The Netherlands. Neuromuscul Disord 23, 1044-1055 (2013).
50. Z. Wei, W. Zhang, H. Fang, Y. Li, X. Wang, esATAC: an easy-to-use systematic pipeline for ATAC-seq data analysis. Bioinformatics 34, 2664-2665 (2018).
51. M. D. Robinson, D. J. McCarthy, G. K. Smyth, edgeR: a Bioconductor package for differential expression analysis of digital gene expression data. Bioinformatics 26, 139-140 (2010).

### Example 3: Molecular Indexing of Proteins by Self-Assembly (MIPSA) Identifies Neutralizing Type I and Type III Interferon Autoantibodies in Severe COVID-19

Unbiased analysis of antibody binding specificities can provide important insights into health and disease states. We and others have utilized programmable phage display libraries to identify novel autoantibodies, characterize anti-viral immunity and profile allergen-specific IgE antibodies.¹⁻⁴ While phage display has been useful for these and many other applications, most protein-protein, protein-antibody and protein-small molecule interactions require a degree of conformational structure that is not captured by bacteriophage displayed peptide libraries. Profiling conformational protein interactions at proteome scale has traditionally relied on protein microarray technologies. Protein microarrays, however, tend to suffer from high per-assay cost, and a myriad of technical artifacts, including those associated with the high throughput expression and purification of proteins, the spotting of proteins onto a solid support, the drying and rehydration of arrayed proteins, and the slide-scanning fluorescence imaging-based readout.^{5,6} Alternative approaches to protein microarray production and storage have been developed (e.g. Nucleic Acid-Programmable Protein Array, NAPPA⁷ or single-molecule PCR-linked in vitro expression, SIMPLEX⁸), but a robust, scalable, and cost-effective alternative has been lacking.

To overcome the limitations associated with array-based profiling of full-length proteins, we previously established a methodology called ParalleL Analysis of Translated Open reading frames (PLATO), which utilizes ribosome display of open reading frame (ORF) libraries.⁹ Ribosome display relies on in vitro translation of mRNAs that lack stop codons, stalling ribosomes at the ends of mRNA molecules in a complex with the nascent proteins they encode. PLATO suffers from several key limitations that have hindered its adoption. An ideal alternative is the covalent conjugation of proteins to short, amplifiable DNA barcodes. Indeed, individually prepared DNA-barcoded antibodies and proteins have been employed successfully in a variety of applications.¹⁰ One particularly attractive protein-DNA conjugation method involves the HaloTag system, which adapts a bacterial enzyme that forms an irreversible covalent bond with halogen-terminated alkane moieties.¹¹ Individual DNA-barcoded HaloTag fusion proteins have been shown to greatly enhance sensitivity and dynamic range of autoantibody detection, compared with traditional ELISA.¹² Scaling individual protein barcoding to entire ORFeome libraries would be immensely valuable, but formidable due to high cost and low throughput. Therefore, a self-assembly approach could provide a much more efficient path to library production.

Here a novel molecular display technology is described, Molecular Indexing of Proteins by Self Assembly (MIPSA), which overcomes key disadvantages of PLATO and other full-length protein array technologies. MIPSA produces libraries of soluble full-length proteins, each uniquely identifiable via covalent conjugation to an amplifiable DNA barcode. Barcodes are introduced upstream of the ribosome binding site (RBS). Partial reverse transcription (RT) of the in vitro transcribed RNA (IVT-RNA) creates a cDNA barcode, which is linked to a haloalkane-labeled RT primer. An N-terminal HaloTag fusion protein is encoded downstream of the RBS, such that in vitro translation results in the intra-complex (“cis”), covalent coupling of the cDNA barcode to the HaloTag and its downstream open reading frame (ORF) encoded protein product. The resulting library of uniquely indexed full-length proteins can be used for inexpensive proteome-wide interaction studies, such as unbiased autoantibody profiling.

Coronavirus disease 2019 (COVID-19), caused by severe acute respiratory syndrome coronavirus 2 (SARS-CoV-2) infection ranges from an asymptomatic course to life-threatening pneumonia and death. A causal link between autoimmunity and severe COVID-19 has been supported by multiple studies.^{13,14} While a diverse array of autoantibodies have been documented,¹⁵ neutralizing type I interferon autoantibodies seem to play a particularly prominent role.^{16,17} Here the utility of the MIPSA platform is investigated by searching for novel autoantibodies in the plasma of patients with severe COVID-19.

### Methods

### Mipsa Destination Vector Construction

The MIPSA vector was constructed using the Gateway pDEST15 vector as a backbone. A gBlock fragment (Integrated DNA Technologies) encoding the RBS, Kozak sequence, N-terminal HaloTag fusion protein, and FLAG tag, followed by an attR1 sequence was cloned into the parent plasmid. A 150 bp poly(A) sequence was also added after attR2 site. The TRIM21 and GAPDH ORF sequences used for characterizing and optimizing the two-component system included native stop codons that were retained in the final MIPSA construct.

### UCI Barcode Library Construction

A 41 nt barcode oligo was generated within a gBlock Gene Fragment (Integrated DNA Technologies) with alternating mixed bases (S: G/C; W: A/T) to produce the following sequence: (SW)₁₈-AGGGA-(SW)₁₈. The sequences flanking the degenerate barcode incorporated the standard PhIP-Seq PCR1 and PCR2 primer binding sites.⁵¹ Eighteen nanograms of the starting UCI library was used to run 40 cycles of PCR to amplify the library and incorporate BglII and PspxI restriction sites. The MIPSA vector and amplified UCI library were then digested with the restriction enzymes overnight, column purified, and ligated at 1:5 vector-to-insert ratio. The ligated MIPSA vector was used to transform electrocompetent One Shot ccdB 2 T1^{R} cells (Thermo Fisher Scientific). Six transformation reactions yielded ˜800,000 colonies to produce the pDEST-MIPSA UCI library.

Human ORFeome Recombination into the pDEST-MIPSA UCI Plasmid Library

150 ng of each pENTR-hORFeome subpool (L1-L5) from the hORFeome v8.1 was individually combined with 150 ng of the pDEST-MIPSA UCI library plasmid and 2 μl of Gateway LR Clonase II mix (Life Technologies) for a total reaction volume of 10 μl. The reaction was incubated overnight at 25° C. The entire reaction was transformed into 50 μl of One Shot OmniMAX 2 T1^{R} chemical competent E. coli (Life Technologies). In aggregate, the transformations yielded ˜120,000 colonies, which is ˜10-fold the complexity of the hORFeome v8.1. Colonies were collected and pooled by scraping, followed by purification of the barcoded pDEST-MIPSA-hORFeome plasmid DNA (human ORFeome MIPSA library) using the Qiagen Plasmid Midi Kit (Qiagen). The human hORFeome v8.1 collection was cloned without stop codons; the displayed proteins may therefore contain poly-lysine C-termini resulting from translation of the poly A tail. A more recent version of the MIPSA destination vector includes a stop codon in frame with recombined ORFs.

### Haloligand Conjugation to RT Oligo and HPLC Purification

100 μg of a 5′ amine modified oligo HL-32_ad (Table 1) was incubated with 75 μl (17.85 μg/μl) of the HaloTag Succinimidyl Ester (02) (Promega Corporation), the HaloLigand, in 0.1 M sodium borate buffer for 6 hours at room temperature following Gu, et al.¹⁹ 3 M NaCl and ice-cold ethanol was added at 10% (v/v) and 250% (v/v), respectively, to the labeling reaction and incubated overnight at −80° C. The reaction was centrifuged for 30 minutes at 12,000×g. The pellet was rinsed once in ice-cold 70% ethanol and air-dried for 10 minutes.

HaloLigand-conjugated RT primer was HPLC purified using a Brownlee Aquapore RP-300 7u, 100×4.6 mm column (Perkin Elmer) using a two-buffer gradient of 0-70% CH₃CN/MeCN (100 mM triethylamine acetate to acetonitrile) over 70 minutes. Fractions corresponding to labeled oligo were collected and lyophilized (FIGS. 15A-15C). Oligos were resuspended at 1 μM (15.4 ng/μl) and stored at −80° C.

### MIPSA Library IVT-RNA Preparation

The human ORFeome MIPSA library plasmid (4 μg) was linearized with the I-SceI restriction endonuclease (New England Biolabs) overnight. The product was column-purified with the NucleoSpin Gel and PCR Clean Up kit (Macherey-Nagel). A 40 μl in vitro transcription reaction using the HiScribe T7 High Yield RNA Synthesis Kit (New England Biolabs) was utilized to transcribe 1 μg of the purified, linearized pDEST-MIPSA plasmid library. The product was diluted with 60 μl molecular biology grade water, and 1 μl of DNAse I was added. The reaction was incubated for another 15 minutes at 37° C. Then 50 μl of 1 M LiCl was added to the solution and incubated at −80° C. overnight. A centrifuge was cooled to 4° C., and the RNA was spun at maximum speed for 30 minutes. The supernatant was removed, and the RNA pellet washed with 70% ethanol. The sample was spun down at 4° C. for another 10 minutes, and the 70% ethanol removed. The pellet was dried at room temperature for 15 minutes, and subsequently resuspended in 100 μl water. To preserve the sample, 1 μl of 40 U/μl RNAseOUT Recombinant Ribonuclease Inhibitor (Life Technologies) was added.

### Mipsa Library IVT-RNA Reverse Transcription and Translation

A reverse transcription reaction was prepared using SuperScript IV First-Strand Synthesis System (Life Technologies). First, 1 μl of 10 mM dNTPs, 1 μl of RNAseOUT (40 U/μl), 4.17 μl of the RNA library (1.5 μM), and 7.83 μl of the HaloLigand-conjugated RT primer (1 μM, Table 1) were combined in a single 14 μl reaction and incubated at 65° C. for 5 minutes followed by a 2-minute incubation on ice. Four microliters of 5×RT buffer, 1 μl of 0.1 M DTT, and 1 μl of SuperScript IV RT Enzyme (200 U/μl) was added to the 14 μl reaction on ice and incubated for 20 minutes at 42° C. A single 20 μl RT reaction received 36 μl of RNAClean XP beads (Beckman Coulter) and was incubated at room temperature for 10 minutes. The beads were collected by magnet and washed five times with 70% ethanol. The beads were air-dried for 10 minutes at room temperature and resuspended in 7 μl of 5 mM Tris-HCl, pH 8.5. The product was analyzed with spectrophotometry to measure the RNA yield. A translation reaction was set up on ice using the PURExpress ΔRibosome Kit (New England Biolabs).⁵² The reaction was modified such that the final concentration of ribosomes was 0.3 μM. For each 10 μl translation reaction, 4.57 μl of the RT reaction was added to 4 μl Solution A, 1.2 μl Factor Mix, and 0.23 μl ribosomes (13.3 μM). This reaction was incubated at 37° C. for two hours, diluted to a total volume of 45 μl with 35 μl 1×PBS, and used immediately or stored at −80° C. after addition of glycerol to a final concentration of 25% (v/v).

Immunoprecipitation of the Translated MIPSA hORFeome Library

5 μl of plasma, diluted 1:100 in PBS, is mixed with the 45 μl of diluted MIPSA library translation reaction (see above) and incubated overnight at 4° C. with gentle agitation. For each IP, a mixture of 5 μl of Protein A Dynabeads and 5 μl of Protein G Dynabeads (Life Technologies) was washed 3 times in 2× their original volume with 1×PBS. The beads were then resuspended in 1×PBS at their original volume, and added to each IP. The antibody capture proceeded for 4 hours at 4° C. Beads were collected on a magnet and washed 3 times in 1×PBS, changing tubes or plates between washes. The beads were then collected and resuspended in a 20 μl PCR master mix containing the T7-Pep2_PCR1_F forward and the T7-Pep2_PCR1_R+ad_min reverse primers (Table 1) and Herculase-II (Agilent). PCR cycling was as follows: an initial denaturing and enzyme activation step at 95° C. for 2 min, followed by 20 cycles of: 95° C. for 20 s, 58° C. for 30 s, and 72° C. for 30 s. The final extension step was performed at 72° C. for 3 minutes. Two microliters of the PCR1 amplification product were used as input to a 20 μl dual-indexing PCR reaction with the PhIP_PCR2 F forward and the PhIP_PCR2_R reverse primers, each containing 10 nt barcodes (i5 and i7, respectively). PCR cycling was as follows: an initial denaturing step at 95° C. for 2 min, followed by 20 cycles of: 95° C. for 20 s, 58° C. for 30 s, and 72° C. for 30 s. The final extension step was performed at 72° C. for 3 min. i5/i7 indexed libraries were pooled and column purified (NucleoSpin columns, Takara). Libraries were sequenced on an Illumina NextSeq 500 using a 1×50 nt SE or 1×75 nt SE protocol. MIPSA_i5_NextSeq_SP and Standard_i7_SP primers were used for i5/i7 sequencing (Table 1) The output was demultiplexed using i5 and i7 without allowing any mismatches.

For quantification of MIPSA experiments by qPCR, the PCR1 product (above) was analyzed as follows. A 4.6 μl of 1:1,000 dilution of the PCR1 reaction was added to 5 μl of Brilliant III Ultra Fast 2×SYBR Green Mix (Agilent), 0.2 μl of 2 μM reference dye and 0.2 μl of 10 μM forward and reverse primer mix (specific to the target UCI). PCR cycling was as follows: an initial denaturing step at 95° C. for 2 min, followed by 45 cycles of: 95° C. for 20 s, 60° C. for 30. Following completion of thermocycling, amplified products were subjected to melt-curve analysis. The qPCR primers for MIPSA immunoprecipitation experiments were: BT2 F and BT2_R for TRIM21, BG4_F and BG4_R for GAPDH, and NT5C1A_F and NT5C1A_R for NT5C1A (Table 1).

### Oligonucleotides

Table 5 provides a list of probes, primers and gRNAs.

### Plasma Samples

All samples were collected from subjects that met protocol eligibility criteria, as described below. All studies protected the rights and privacy of the study participants and were approved by their respective Institutional Review Boards for original sample collection and subsequent analyses.

Pre-pandemic and healthy control plasma samples. All human samples were collected prior to 2017 at the National Institutes of Health (NIH) Clinical Center under the Vaccine Research Center's (VRC)/National Institutes of Allergy and Infectious Diseases (NIAID)/NIH protocol “VRC 000: Screening Subjects for HIV Vaccine Research Studies” (NCT00031304) in compliance with NIAID IRB approved procedures.

COVID-19 Convalescent Plasma (CCP) from non-hospitalized patients. Eligible non-hospitalized CCP donors were contacted by study personnel, as previously described.⁵³ All donors were at least 18 years old and had a confirmed diagnosis of SARS-CoV-2 by detection of RNA in a nasopharyngeal swab sample. Basic demographic information (age, sex, hospitalization with COVID-19) was obtained from each donor; initial diagnosis of SARS-CoV-2 and the date of diagnosis were confirmed by medical chart review.

Severe COVID-19 plasma samples. The study cohort was defined as inpatients who had: 1) a confirmed RNA diagnosis of COVID-19 from a nasopharyngeal swab sample; 2) survival to death or discharge; and 3) remnant specimens in the Johns Hopkins COVID-19 Remnant Specimen Biorepository, an opportunity sample that includes 59% of Johns Hopkins Hospital COVID-19 patients and 66% of patients with length of stay ≥3 days.^{54,55} Patient outcomes were defined by the World Health Organization (WHO) COVID-19 disease severity scale. Samples from severe COVID-19 patients that were included in this study were obtained from 17 patients who died, 13 who recovered after being ventilated, 22 who required oxygen to recover, and 3 who recovered without supplementary oxygen. This study was approved by the JHU Institutional Review Board (IRB00248332, IRB00273516), with a waiver of consent because all specimens and clinical data were de-identified by the Core for Clinical Research Data Acquisition of the Johns Hopkins Institute for Clinical and Translational Research; the study team had no access to identifiable patient data.

Sjögren's Syndrome and Inclusion body myositis (IBM) plasma samples. Sjögren's syndrome samples were collected under protocol NA_00013201. All patients were >18 years old and gave informed consent. IBM patient samples were collected under protocol IRB00235256. All patients met ENMC 2011 diagnostic criteria⁵⁶ and provided informed consent.

### Immunoblot Analysis

Laemmli buffer containing 5% β-ME was added to samples, boiled for 5 min, and analyzed on NuPage 4-12% Bis-Tris polyacrylamide gels (Life Technologies). Following transfer to PVDF membranes, blots were blocked in 20 mM Tris-buffered saline, pH 7.6, containing 0.1% Tween 20 (TBST) and 5% (wt/vol) non-fat dry milk for 30 minutes at room temperature. Blots were subsequently incubated overnight at 4° C. with primary anti-FLAG antibody (#F3165, MilliporeSigma) at 1:2,000 (v/v), followed by a 4-hour incubation at room temperature in anti-mouse IgG, HRP-linked secondary antibody (#7076, Cell Signaling) at 1:4,000 (v/v).

### Construction of the UCI-ORF Dictionary

The Nextera XT DNA Library Preparation kit (Illumina) was used for tagmentation of 150 ng of the pDEST-MIPSA hORFeome plasmid library to yield the optimal size distribution centered around 1.5 kb. Tagmented libraries were amplified using Herculase-II (Agilent) with T7-Pep2_PCR1_F forward and Nextera Index 1 Read primer. PCR cycling was as follows: an initial denaturing step at 95° C. for 2 minutes, followed by 30 cycles of: 95° C. for 20 s, 53.5° C. for 30 s, 72° C. for 30 s. A final extension step was performed at 72° C. for 3 minutes. PCR reactions were run on a 1% agarose gel followed by excision of ˜1.5 kb products and purification using the NucleoSpin Gel and PCR Clean-up columns (Macherey-Nagel). The purified product was then amplified for another 10 cycles with PhIP_PCR2_F forward and P7.2 reverse primers (see Table 1 for list of primer sequences). The product was gel-purified and sequenced on a MiSeq (Illumina) using the T7-Pep2.2_SP_subA primer for read 1 and the MISEQ_MIPSA_R2 primer for read 2. Read 1 was 60 bp long to capture the UCIs. The first index read, I1, was substituted with a 50 bp read into the ORF. 12 was used to identify the i5 index for sample demultiplexing.

The hORFeome v8.1 DNA sequences were truncated to the first 50 nt, and the ORF names corresponding to non-unique sequences were concatenated with a “|” delimiter. The demultiplexed output of the 50 nt R2 (ORF) read from an Illumina MiSeq was aligned to the truncated human ORFeome v8.1 library using the Rbowtie2 package with the following parameters: options=“-a --very-sensitive-local”.⁵⁷ The unique FASTQ identifiers were then used to extract corresponding sequences from the 60 bp R1 (UCI) read. Those sequences were then truncated using the 3′ anchor ACGATA, and sequences that did not have the anchor were removed. Additionally, any truncated R1 sequences that had fewer than 18 nucleotides were removed. The ORF sequences that still had a corresponding UCI post-filtering were retained using the FASTQ identifier. The names of ORFs that had the same UCI were concatenated with a “&” delimiter, and this final dictionary was used to generate a FASTA alignment file composed of ORF names and UCI sequences.

### Informatic Analysis of MIPSA Sequencing Data

Illumina output FASTQ files were truncated using the constant ACGAT anchor sequence following all UCI sequences. Next, perfect match alignment was used to map the truncated sequences to their linked ORFs via the UCI-ORF lookup dictionary. A read count matrix was constructed, in which rows correspond to individual UCIs and columns correspond to samples. The edgeR software package⁵⁸ was used which, using a negative binomial model, compares the signal detected in each sample against a set of negative control (“mock”) IPs that were performed without plasma, to return a maximum likelihood fold-change estimate and a test statistic for each UCI in every sample, thus creating fold-change and −log 10(p-value) matrices. By comparison of EdgeR output data from replicate IPs, it was established that significantly enriched UCIs (“hits”) should require a read count of at least 15, a p-value less than 0.001, and a fold change of at least 3. Hits fold-change matrices report the fold-change value for “hits” and report a “1” for UCIs that are not hits.

### Protein Sequence Similarity

To evaluate sequence homology among proteins in the hORFeome v8.1 library, a blastp alignment was used to compare each protein sequence against all other library members (parameters: “-outfmt 6 -evalue 100 -max_hsps 1 -soft_masking false-word_size 7 -max_target_seqs 100000”). To evaluate sequence homology among reactive peptides in the human 90-aa phage display library, the epitopefindr⁵⁹ software was employed.

### Phage ImmunoPrecipitation Sequencing (PhIP-Seq) Analyses P

hIP-Seq was performed according to a previously published protocol.⁵¹ Briefly, 0.2 μl of each plasma was individually mixed with the 90-aa human phage library and immunoprecipitated using protein A and protein G coated magnetic beads. A set of 6-8 mock immunoprecipitations (no plasma input) were run on each 96 well plate. Magnetic beads were resuspended in PCR master mix and subjected to thermocycling. A second PCR reaction was employed for sample barcoding. Amplicons were pooled and sequenced on an Illumina NextSeq 500 instrument using a 1×50 nt SE or 1×75 nt SE protocol. PhIP-Seq with the human library was used to characterize autoantibodies in a collection of plasma from healthy controls. For fair comparison to the severe COVID-19 cohort, the minimum sequencing depth that would have been required to detect the IFN-λ3 reactivity in both of the positive individuals was first determined. Only then were the 423 data sets from the healthy cohort were considered with sequencing depth greater than this minimum threshold. None of these 423 individuals were found to be reactive to any peptide from IFN-λ3.

### Type I/III Interferon Neutralization Assay

IFN-α2 (catalog no. 11100-1), IFN-λ1 (catalog no. 1598-IL-025) and IFN-λ3 (catalog no. 5259-IL-025) were purchased from R&D Systems. Twenty microliters of plasma were incubated for 1 hour at room temperature with either 100 U/ml IFN-α2 or 1 ng/ml IFN-23, and 180 μl DMEM in a total volume of 200 μl before addition into 7.5×10⁴ A549 cells in 48-well tissue culture plates. After 4-hour incubation, the cells were washed with 1×PBS and cellular mRNA was extracted and purified using RNeasy Plus Mini Kit (Qiagen). Six hundred nanograms of extracted mRNA was reverse transcribed using the SuperScript III First-Strand Synthesis System (Life Technologies) and diluted 10-fold for qPCR analysis on a QuantStudio 6 Flex System (Applied Biosystems). PCR consisted of 95° C. for 3 minutes, followed by 45 cycles of the following: 95° C. for 15 seconds and 60° C. for 30 seconds. MX1 expression was chosen as a measure of cell stimulation by the interferons, and the relative mRNA expression was normalized to GAPDH expression. The qPCR primers for GAPDH and MX1 were obtained from Integrated DNA Technologies (Table 1). Anti-hIFN-α2-IgG (cat #mabg-hifna-3) and anti-hIL-28b-IgG (cat #mabg-hil28b-3) were purchased from InvivoGen. Manufacturer's note about mabg-hifna-3: “This antibody reacts with hIFN-α1, hIFN-α2, hIFN-α5, hIFN-α8, hIFN-α14, hIFN-α16, hIFN-α17 and hIFN-α21; it reacts very weakly with hIFN-α4 and IFN-α10; it does not react with hIFN-α6 or hIFN-α7.” The Manufacturer's note about mabg-hil28b-3: “Reacts with human IL-28A and human IL-28B.”

### Results

### Development of the MIPSA System

The MIPSA Gateway Destination vector for E. coli cell free translation contains the following key elements: a T7 RNA polymerase transcriptional start site, an isothermal unique clonal identifier (“UCI”) barcode sequence, an E. coli ribosome binding site (RBS), an N-terminal HaloTag fusion protein (891 nt), recombination sequences for ORF insertion, and a homing endonuclease (I-SceI) site for plasmid linearization. A recombined ORF-containing pDEST-MIPSA plasmid is shown in FIG. 1A.

It was first sought to establish a library of pDEST-MIPSA plasmids containing stochastic, isothermal UCIs located between the transcriptional start site and the ribosome binding site. A degenerate oligonucleotide pool was synthesized, comprising melting temperature (Tm) balanced sequences: (SW)₁₈-AGGGA-(SW)₁₈, where S represents an equal mix of C and G, while W represents an equal mix of A and T (FIG. 1B). It was reasoned that this inexpensive pool of sequences would (i) provide sufficient complexity (2³⁶˜7×10¹⁰) for unique ORF labeling, (ii) amplify without distortion, and (iii) serve as ORF-specific forward and reverse qPCR primer binding sites for measurement of individual UCIs of interest. The degenerate oligonucleotide pool was amplified by PCR, restriction cloned into the MIPSA destination vector, and transformed into E. coli (Methods). About 800,000 transformants were scraped off selection plates to obtain the pDEST-MIPSA UCI plasmid library. ORFs encoding the housekeeping gene glyceraldehyde-3-phosphate dehydrogenase (GAPDH) and a known autoantigen, tripartite motif containing-21 (TRIM21, commonly known as Ro52), were separately recombined into the pDEST-MIPSA UCI plasmid library. Individually barcoded GAPDH and TRIM21 clones were isolated, sequenced, and used in the following experiments.

The MIPSA procedure involves RT of the UCI using a succinimidyl ester (02)-haloalkane (HaloLigand)-conjugated RT primer (FIGS. 6A-6C). The bound RT primer should not interfere with the assembly of the E. coli ribosome and initiation of translation, but should be sufficiently proximal such that coupling of the HaloLigand-HaloTag-protein complex might hinder additional rounds of translation. A series of RT primers were assessed that anneal at distances ranging from −42 nucleotides to −7 nucleotides relative to the 3′ end of the ribosome binding site (FIG. 1D). Based on the yield of protein product from mRNA saturated with primers at these differing locations, the −32 position was selected as it did not interfere with translation efficiency (FIG. 1E). In contrast, RT from primers located within 20 nucleotides of the RBS diminished or abolished protein translation, in agreement with the estimated footprint of assembled 70S E. coli ribosomes, which have been shown to protect a minimum of 15 nucleotides of mRNA.¹⁸

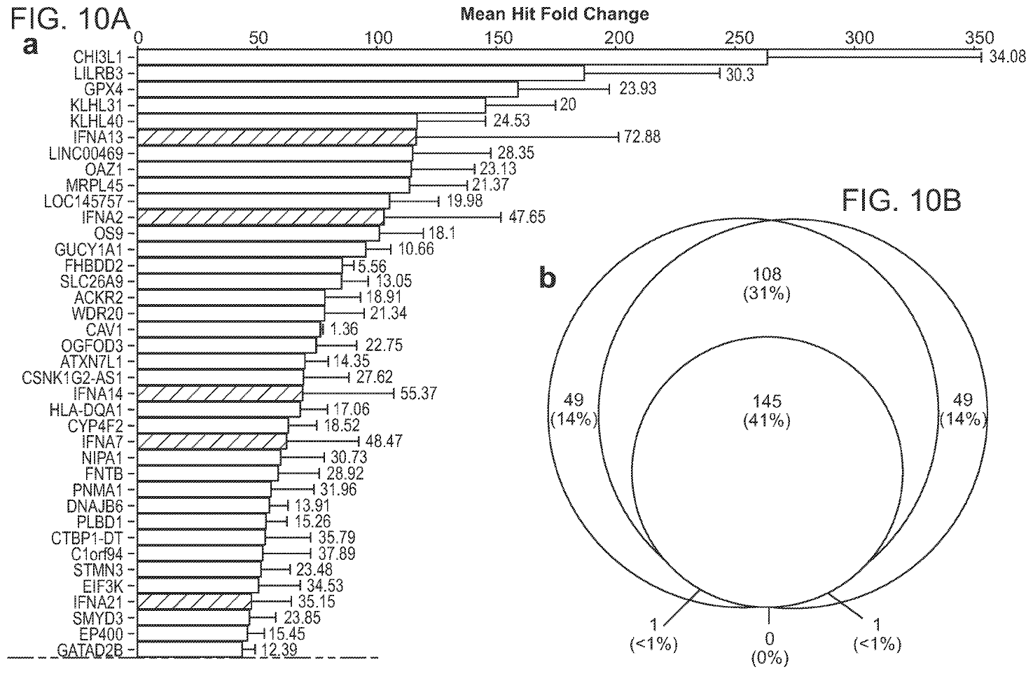

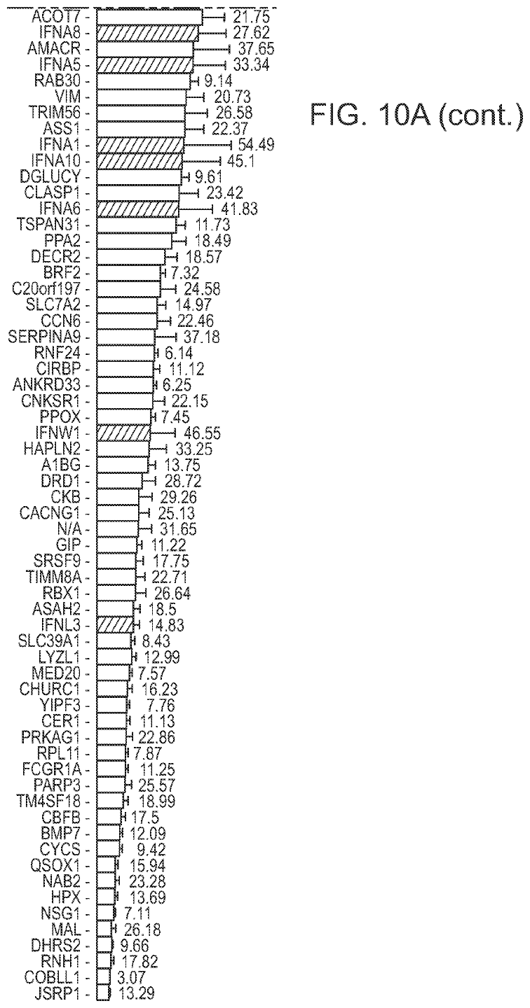

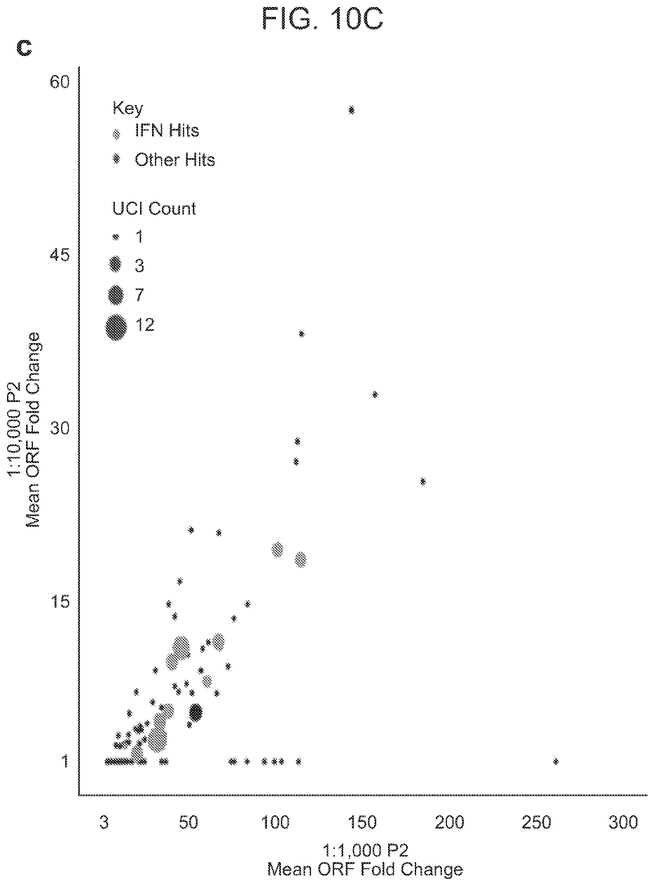

The ability of SuperScript IV to perform RT from a primer labeled with the HaloLigand at its 5′ end, and the ability of the HaloTag-TRIM21 protein to form a covalent bond with the HaloLigand-conjugated primer during the translation reaction was assessed. HaloLigand conjugation and purification followed Gu et al. (Methods, FIGS. A-15C).¹⁹ Either an unconjugated RT primer or a HaloLigand-conjugated RT primer was used for RT of the barcoded HaloTag-TRIM21 mRNA. The translation product was then immuno-captured (i.e., immunoprecipitated, “IPed”) with plasma from a healthy donor or plasma from a TRIM21 (Ro52) autoantibody-positive patient with Sjögren's Syndrome (SS), using protein A and protein G coated magnetic beads. The SS plasma efficiently IPed the TRIM21 protein, regardless of RT primer conjugation, but only pulled down the TRIM21 UCI when the HaloLigand-conjugated primer was used in the RT reaction (FIGS. 10F-10G).

### Assessing Levels of Cis Versus Trans UCI Barcoding

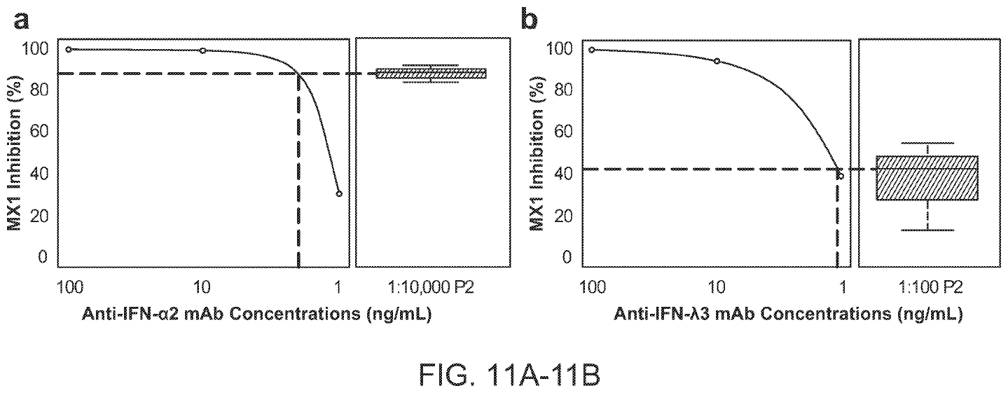

While the previous experiment indicated that indeed the HaloLigand does not impede RT priming, and that the HaloTag can form a covalent bond with the HaloLigand during the translation reaction, it did not elucidate the amount of cis (intra-complex, desirable) versus trans (inter-complex, undesirable) HaloTag-UCI conjugation (FIGS. 16A-16C). Here, “intra-complex” is defined as conjugation to the UCI that is associated with the same RNA molecule encoding the protein. To measure the amount of cis and trans HaloTag-UCI conjugation, GAPDH and TRIM21 mRNAs were separately reverse transcribed (using HaloLigand-conjugated primer) and then either mixed 1:1 or kept separate for in vitro translation. As expected, translation of the mixture produced roughly equivalent amounts of each protein compared to the individual translations (FIG. 8). SS plasma specifically IPed TRIM21 protein regardless of translation condition (FIG. 8, IPed fraction). However, it was noted that while the SS IPs contained high levels of the TRIM21 UCI, as intended, more of the GAPDH UCI was pulled down by the SS plasma compared to that by the HC plasma when the mRNA was mixed prior to translation. This indicates that indeed some amount of trans barcoding occurs (FIG. 11A). We estimate that ˜50% of the protein is cis-barcoded, with the remaining 50% trans-barcoded protein equally conjugated to both UCIs. Thus, in this two-component system, 25% of the TRIM21 protein is conjugated to the GAPDH UCI (FIGS. 16A-16C).

In the setting of a complex library, even if ˜50% of each protein is trans barcoded, this side product should be associated with a low level of randomly sampled UCIs. We tested this using a mock MIPSA library, composed of 100-fold excess of a second GAPDH clone, which was combined with a 1:1 mixture of the first GAPDH and TRIM21 clones (FIG. 2B).

### Establishing and Deconvoluting a Stochastically Barcoded Human ORFeome MIPSA Library

The sequence-verified human ORFeome (hORFeome) v8.1 is composed of 12,680 clonal ORFs mapping to 11,437 genes in the Gateway Entry plasmid (pDONR223).²⁰ Five subpools of the library were created, each composed of ˜2,500 similarly sized ORFs. Each of the five subpools was separately recombined into the pDEST-MIPSA UCI plasmid library and transformed to obtain ˜10-fold ORF coverage (˜25,000 clones per subpool). Each subpool was assessed via Bioanalyzer electrophoresis, sequencing of ˜20 colonies, and Illumina sequencing of the combined superpool. The TRIM21 plasmid was spiked into the superpooled hORFeome library at 1:10,000-comparable to a typical library member. The SS IP experiment was then performed on the hORFeome MIPSA library, using sequencing as a readout. The read counts from all UCIs in the library, including the spiked-in TRIM21, are shown for the SS IP versus the average of 8 mock IPs in FIG. 11C. Reassuringly, the SS autoantibody-dependent enrichment of TRIM21 (17-fold) was similar to the model system (FIG. 11D). See Informatic analysis of MIPSA sequencing data in the Methods section for a description of the analytical pipeline for sequencing data.

Next, a system was established for creating a UCI-ORF lookup dictionary, using tagmentation and sequencing (FIG. 3A). Sequencing the 5′ 50 nt of the ORF inserts detected 11,076 of the 11,887 unique 5′ 50 nt library sequences. Of the 153,161 UCIs detected, 82.9% (126,975) were found to be associated with a single ORF (termed a “monospecific UCI”). Each ORF was uniquely associated with a median of 9 (ranging from 0 to 123) monospecific UCIs (FIG. 3B). Importantly, an ensemble of monospecific UCIs with consistent behavior can provide additional, strong support for the reactivity of their associated ORF. A weak, inverse correlation between UCI number and ORF size was noted, which most likely reflects the less efficient recombination of larger ORF-containing plasmids in the pooled recombination reaction. After aggregation of the read counts corresponding to each ORF, over 99% of the represented ORFs were present within a 10-fold difference of the median ORF abundance (FIG. 3C). Taken together, these data indicate that a uniform library of 11,076 stochastically indexed human ORFs was established, and defined a lookup dictionary for downstream analyses. FIG. 3D shows UCI read counts of an SS IP versus the average of 8 mock IPs, and the 47 dictionary-decoded GAPDH monospecific UCIs (corresponding to two GAPDH isoforms present in the hORFeome library) appearing along the y=x diagonal as expected. To avoid ambiguity, any UCI associated with more than a single ORF was excluded from further analyses.

Unbiased MIPSA analysis of autoantibodies associated with severe COVID-19 Several recent reports have described elevated autoantibody reactivities in patients with severe COVID-19.²¹⁻²⁵ MIPSA was used with the human ORFeome library for unbiased identification of autoreactivities in the plasma of 55 severe COVID-19 patients, defined here based only on hospital admission, since the availability of clinical meta-data was incomplete. For comparison, MIPSA was used to detect autoreactivities in plasma from 10 healthy donors and 10 COVID-19 convalescent plasma donors who had not been hospitalized (Table 2). As was done previously for Phage ImmunoPrecipitaiton Sequencing (PhIP-Seq) analyses, each sample was compared to a set of 8 “mock IPs”, which contained all reaction components except for plasma, and were run on the same plate. Comparison to mock IPs accounts for bias in the library and background binding. The informatic pipeline used to detect antibody-dependent reactivity (Methods) yielded a median of 5 (ranging from 2 to 9) false positive UCI hits per mock IP. IPs using plasma from severe COVID-19 patients, however, yielded a mean of 83 reactive proteins among severe COVID-19 patients, which was significantly more than the mean of 64 reactive proteins among healthy pre-pandemic controls and significantly more than the mean of 62 reactive proteins among recovered individuals after mild to moderate COVID-19 (p=0.02 and p=0.05, respectively, one tailed t-test; FIG. 4A).

Proteins were examined in the severe COVID-19 IPs that had at least two reactive UCIs (in the same IP), which were reactive in at least one severe patient, and that were not reactive in more than one control (healthy or mild/moderate convalescent plasma). Proteins were excluded if they were reactive in a single severe patient and a single control. The 103 proteins that met these criteria are shown in the cluster map of FIG. 4B. Fifty one of the 55 severe COVID-19 patients exhibited reactivity to at least one of these proteins. Co-occurring protein reactivities in multiple individuals was noted, the vast majority of which lack homology by protein sequence alignment. Table 4 provides summary statistics about these reactive proteins, including whether they are previously defined autoantigens according to the human autoantigen database AAgAtlas 1.0.²⁶ Proteins were included if they had at least two reactive UCIs in at least one severe patient and were not reactive in more than one control (healthy or mild/moderate convalescent plasma). Proteins were not included if they were reactive in a single severe patient and a single control. Each row corresponds to a single UCI, organized by protein in alphabetical order (gene symbol provided to left of underscore). Each column is an individual COVID-19 patient. If the UCI read counts were not significantly enriched versus the mock IPs, it is reported as “1”. If the UCI read counts were significantly enriched versus mock IPs, the fold-change estimate (from EdgeR) is provided.

One notable autoreactivity cluster (Table 4, cluster #5) includes 5′-nucleotidase, cytosolic 1A (NT5C1A), which is highly expressed in skeletal muscle and is the most well-characterized autoantibody target in inclusion body myositis (IBM). Multiple UCIs linked to NT5C1A were significantly increased in 3 of the 55 severe COVID-19 patients (5.5%). NT5C1A autoantibodies have been reported in up to 70% of IBM patients ¹, in ˜20% of Sjogren's Syndrome (SS) patients, and in up to ˜5% of healthy donors.²⁷ The prevalence of NT5C1A reactivity in the severe COVID-19 cohort is therefore not necessarily elevated. However, we wondered whether MIPSA would be able to reliably distinguish between healthy donor and IBM plasma based on NT5C1A reactivity. Plasma from 10 healthy donors and 10 IBM patients was used, the latter of whom were selected based on NT5C1A seropositivity determined by PhIP-Seq.¹ The clear separation of patients from controls in this independent cohort suggests that MIPSA may indeed have utility in clinical diagnostic testing using either UCI-specific qPCR or library sequencing, which were tightly correlated readouts (FIG. 4C).

### Type I and Type III Interferon-Neutralizing Autoantibodies in Severe COVID-19 Patients

Neutralizing autoantibodies targeting type I interferons alpha (IFN-α) and omega (IFN-ω) have been associated with severe COVID-19.^{15,22,28} All type I interferons except IFN-α16 are represented in the human MIPSA ORFeome library and annotated in the lookup dictionary. IFN-α4, IFN-α17, and IFN-α21 are indistinguishable by the first 50 nucleotides of their encoding ORF sequences, and thus analyzed as a single ORF. Two of the severe COVID-19 patients (P1 and P2) in this cohort (3.6%) exhibited dramatic type I IFN autoreactivity (49 and 46 type I interferon UCIs, across 11 distinct ORFs corresponding to many IFN-α's and IFN-ω FIGS. 5A, 5B). The extensive co-reactivity of these proteins is likely attributable to their sequence homology (FIG. 9). By requiring at least 2 reactive IFN UCIs to be considered positive, two additional severe COVID-19 plasma (P3 and P4) were identified with detectable levels of IFN-α reactivity, each with only 2 reactive IFN-α UCIs. Fifty percent of these four IFN-α autoreactive patients died, versus about 30% of the remaining cohort. Interestingly, one additional plasma (P5) precipitated no UCIs from any type I or II interferons, but five UCIs from the type III interferon IFN-λ3 (FIGS. 5C, 5D). This patient also died of COVID-19. No additional interferon autoreactivities were detected among the severe COVID-19 patients. None of the healthy or non-hospitalized COVID-19 controls were positive for 2 or more interferon UCIs.

The performance of MIPSA using P2 plasma was assessed, which neutralizes both type I and III interferons. MIPSA was run on P2 plasma in triplicate, yielding a high level of assay reproducibility (FIGS. 19A, 19B), both in consistent detection of hits and low coefficients of variation (mean CV=22%). The linearity of the assay was assessed by diluting P2 plasma 10-fold into a healthy plasma and then performing MISPA again. The results demonstrate a consistent decrease in signal among the reactivities (mean of 5.4-fold for reactive interferons), and loss of detection of some hits, particularly of ORFs with single reactive UCIs.

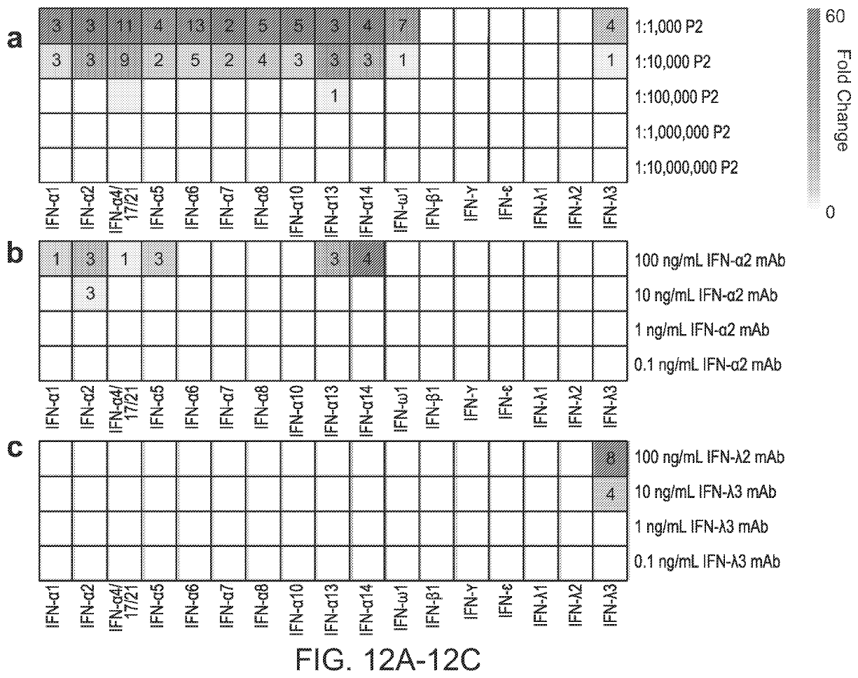

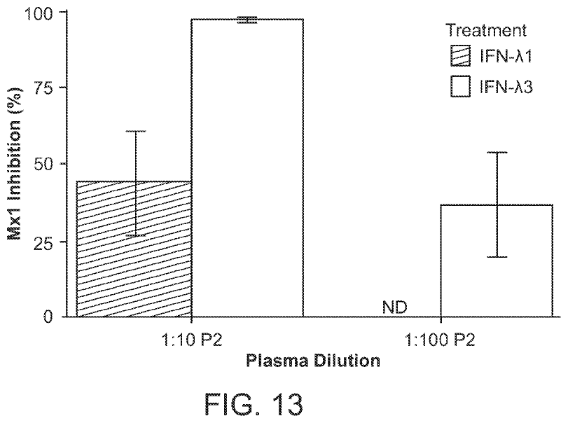

Incubation of A549 human adenocarcinomatous lung epithelial cells with 100 U/ml IFN-α or 1 ng/ml of IFN-λ3 for 4 hours in serum-free medium results in a robust upregulation of the IFN-response gene MX1 by ˜1,000-fold and ˜100-fold, respectively. Pre-incubation of IFN-α2 with plasma P1, P2, or P3 completely abolished MX1 upregulation (FIG. 5E). The plasma with the weakest IFN-α reactivity by MIPSA, P4, only partially neutralized the cytokine. Neither HC nor P5 plasma had any effect on the response of A549 cells to IFN-α2 treatment. However, pre-incubation of the IFN-λ3 cytokine with the MIPSA-positive plasma, P2 and P5, ablated the interferon response (FIG. 5F). None of the other plasma (HC, P1, P3, or P4) had any effect on the response of A549 cells to IFN-λ3. By comparison against titration curves using IFN-α2 and IFN-λ3 monoclonal antibodies, a serial titration using patient P2 plasma in triplicate indicated circulating levels of these autoantibodies to be ˜20 μg/ml and ˜100 ng/ml, respectively (FIGS. 11A, 11B). MIPSA analysis of the serially diluted IFN-α2 mAb revealed broad IFN-α cross-recognition, but mutually exclusive binding of the mAbs to the appropriate type I or type III interferon (FIG. 12A, 12B). Importantly, we noted that loss of MIPSA detection sensitivity corresponded to the same or greater plasma dilutions at which IFN-α2 and IFN-λ3 neutralization activities were also lost. Finally, the titer of P2's autoantibodies exhibited at least a 10-fold preference for IFN-λ3 neutralization over IFN-λ1 neutralization (FIG. 13). In summary, MIPSA-based autoantibody profiling of this severe COVID-19 cohort identified strongly neutralizing IFN-α autoantibodies in 7.3% of patients and strongly neutralizing IFN-λ3 autoantibodies in 3.6% of patients, with a single patient (1.8%) harboring both autoreactivities.

It was then determined whether Phage ImmunoPrecipitation Sequencing (PhIP-Seq) with a 90-aa human peptidome library²⁹ might also detect interferon antibodies in this cohort. PhIP-Seq detected IFN-α reactivity in plasma from P1 and P2, although to a much lesser extent (FIG. 5G). The two weaker IFN-α reactivities detected by MIPSA in the plasma of P3 and P4 were both missed by PhIP-Seq. PhIP-Seq identified a single additional weakly IFN-α reactive sample, which was negative by MIPSA (not shown). Both technologies detected type III interferon autoreactivity (directed exclusively at IFN-λ3). PhIP-Seq data was used to narrow the location of a dominant epitope in these type I and type III interferon autoantigens (FIG. 5H for IFN-α; amino acid position 45-135 for IFN-λ3).

The prevalence of the previously unreported IFN-λ3 autoreactivity in the general population, and whether it might be increased among patients with severe COVID-19 was assessed. PhIP-Seq was previously used to profile the plasma of 423 healthy controls, none of whom were found to have detectable IFN-λ3 autoreactivity.³⁰ These data suggest that IFN-λ3 autoreactivity is likely to be more frequent among individuals with severe COVID-19. This is the first report describing neutralizing IFN-λ3 autoantibodies, and therefore provides a potentially novel pathogenic mechanism contributing to life-threatening COVID-19 in a subset of patients.

### Discussion

Here a novel molecular display technology was presented for full length proteins, which provides key advantages over protein microarrays, PLATO, and alternative techniques. MIPSA utilizes self-assembly to produce a library of proteins, linked to relatively short (158 nt) single stranded DNA barcodes via the 25 kDa HaloTag domain. This compact barcoding approach will likely have numerous applications not accessible to alternative display formats with bulky linkage cargos (e.g. yeast, bacteria, viruses, phage, ribosomes, mRNAs, cDNAs). Indeed, individually conjugating minimal DNA barcodes to proteins, especially antibodies and antigens, has already proven useful in several settings, including CITE-Seq,³¹ LIBRA-seq,³² and related methodologies.³³ At proteome scale, MIPSA will enable unbiased analyses of protein-antibody, protein-protein, and protein-small molecule interactions, as well as studies of post-translational modification, such as hapten modification studies³⁴ or protease activity profiling³⁵, for example. Key advantages of MIPSA include its high throughput, low cost, simple sequencing library preparation, inherent compatibility with PhIP-Seq, and stability of the protein-DNA complexes (important for manipulation and storage of display libraries). Importantly, MIPSA can be immediately adopted by standard molecular biology laboratories, since it does not require specialized training or instrumentation, simply access to a high throughput DNA sequencing instrument or facility.

### Autoantibodies Detected in Severe COVID-19 Patients Using MIPSA

Neutralizing IFN-α/ω autoantibodies have been described in patients with severe COVID-19 disease and are presumed to be pathogenic.²² These likely pre-existing autoantibodies, which occur very rarely in the general population, block restriction of viral replication in cell culture, and are thus likely to interfere with disease resolution. This discovery paved the way to identifying a subset of individuals at risk for life-threatening COVID-19 and proposed therapeutic use of interferon beta in this population of patients. In this study, MIPSA identified two individuals with extensive reactivity to the entire family of IFN-α cytokines. Indeed, plasma from both individuals, plus two individuals with weaker IFN-α reactivity detected by MIPSA, robustly neutralized recombinant IFN-α2 in a lung adenocarcinomatous cell culture model.

Type III IFNs (IFN-λ, also known as IL-28/29) are cytokines with potent anti-viral activities that act primarily at barrier sites. The IFN-λR1/IL-10RB heterodimeric receptor for IFN-λ is expressed on lung epithelial cells and is important for the innate response to viral infection. Mordstein et al., determined that in mice, IFN-λ diminished pathogenicity and suppressed replication of influenza viruses, respiratory syncytial virus, human metapneumovirus, and severe acute respiratory syndrome coronavirus (SARS-CoV-1).³⁶ It has been proposed that IFN-λ exerts much of its antiviral activity in vivo via stimulatory interactions with immune cells, rather than through induction of the antiviral cell state.³⁷ However, IFN-λ has been found to robustly restrict SARS-CoV-2 replication in primary human bronchial epithelial cells³⁸, primary human airway epithelial cultures³⁹, and primary human intestinal epithelial cells⁴⁰. Collectively, these studies suggest multifaceted mechanisms by which neutralizing IFN-λ autoantibodies may exacerbate SARS-CoV-2 infections.

Among 55 severe COVID-19 patients, MIPSA detected two individuals with IFN-λ3 reactive autoantibodies. The same autoreactivities were also detected using PhIP-Seq. We tested the IFN-λ3 neutralizing capacity of these patients' plasma, observing near complete ablation of the cellular response to the recombinant cytokine (FIG. 5F). These data propose that IFN-λ3 autoreactivity is a new, potentially pathogenic mechanism contributing to severe COVID-19 disease.

Casanova, et al. did not detect any type III IFN neutralizing antibodies among 101 individuals with type I IFN autoantibodies tested.²² In this study, one of the four IFN-α autoreactive individuals (P2, a 22-year-old male) also harbored autoantibodies that neutralized IFN-λ3. It is possible that this co-reactivity is extremely rare and thus not represented in the Casanova cohort. Alternatively, it is possible that the differing assay conditions exhibit different detection sensitivity. Whereas Casanova, et al. cultured A549 cells with IFN-λ3 at 50 ng/ml without plasma preincubation, here A549 cells were cultured with IFN-λ3 at 1 ng/ml after pre-incubation with plasma for one hour. Their readout of STAT3 phosphorylation may also provide different detection sensitivity compared to the upregulation of MX1 expression. A larger study should determine the true frequency of these reactivities in severe COVID-19 patients and matched controls. Here, detection of strongly neutralizing IFN-α and IFN-λ3 autoantibodies in 4 (7.3%) and 2 (3.6%) individuals is reported, respectively, in a cohort of 55 patients with severe COVID-19. IFN-λ3 autoantibodies were not detected via PhIP-Seq in a larger cohort of 423 healthy controls collected prior to the pandemic.

Exogenously administered Type III interferons have been proposed as a therapeutic for SARS-CoV-2 infection,^{39,41-45} and there are currently three ongoing clinical trials to test pegylated IFN-λ1 for efficacy in reducing morbidity and mortality associated with COVID-19 (ClinicalTrials.gov Identifiers: NCT04343976, NCT04534673, NCT04344600). One recently completed double-blind, placebo-controlled trial, NCT04354259, reported a significant reduction by 2.42 log copies per ml of SARS-CoV-2 at day 7 among mild to moderate COVID-19 patients in the outpatient setting (p=0.0041).⁴⁶ Future studies will determine whether anti-IFN-λ3 autoantibodies are pre-existing or arise in response to SARS-CoV-2 infection, and how often they also cross-neutralize IFN-λ1. Based on neutralization data from P2 (FIG. 13) and sequence alignment of IFN-λ1 and IFN-λ3 (˜29% homology, FIG. 9), cross-neutralization is expected to be rare, raising the possibility that patients with neutralizing IFN-λ3 autoantibodies may derive benefit from pegylated IFN-λ1 treatment.

While clusters of uncharacterized autoreactivities were observed in multiple individuals, it is not clear what role, if any, they may play in severe COVID-19. In larger scale studies, we expect that patterns of co-occurring reactivity, or reactivities towards proteins with related biological functions, may ultimately define new autoimmune syndromes associated with severe COVID-19.

### Complementarity of MIPSA and PhIP-Seq

Display technologies frequently complement one another but may not be amenable to routine simultaneous use. MIPSA is more likely than PhIP-Seq to detect antibodies directed at conformational epitopes on proteins expressed well in vitro. This was exemplified by the robust detection of interferon alpha autoantibodies via MIPSA, which were less sensitively detected via PhIP-Seq. PhIP-Seq, on the other hand, is more likely to detect antibodies directed at less conformational epitopes contained within proteins that are either absent from an ORFeome library or cannot be expressed well in cell-free lysate. Because MIPSA and PhIP-Seq naturally complement one another in these ways, we designed the MIPSA UCI amplification primers to be the same as those we have used for PhIP-Seq. Since the UCI-protein complex is stable-even in phage preparations-MIPSA and PhIP-Seq can readily be performed together in a single reaction, using a single set of amplification and sequencing primers. The compatibility of these two display modalities lowers the barrier to leveraging their synergy.

### Variations of the MIPSA System

A key aspect of MIPSA involves the conjugation of a protein to its associated UCI in cis, compared to another library member's UCI in trans. Here covalent conjugation was utilized via the HaloTag/HaloLigand system, but others could work as well. For instance, the SNAP-tag (a 20 kDa mutant of the DNA repair protein 06-alkylguanine-DNA alkyltransferase) forms a covalent bond with benzylguanine (BG) derivatives.⁴⁷ BG could thus be used to label the RT primer in place of the HaloLigand. A mutant derivative of the SNAP-tag, the CLIP-tag, binds O2-benzylcytosine derivatives, which could also be adapted to MIPSA.⁴⁸

The rate of HaloTag maturation and ligand binding is critical to the relative yield of cis versus trans UCI conjugation. A study by Samelson et al. determined that the rate of HaloTag protein production is about fourfold higher than the rate of HaloTag functional maturation.⁴⁹ Considering a typical protein size is <1,000 amino acids in the ORFeome library, these data predict that most proteins should be released from the ribosome before HaloTag maturation, and thus before cis HaloLigand conjugation could occur, thereby favoring unwanted trans barcoding. However, here it was observed that ˜50% of protein-UCI conjugates are formed in cis, thereby enabling excellent assay performance in the setting of a complex library. During optimization experiments, the rate of cis barcoding was found to be slightly improved by excluding release factors from the translation mix, which stalls ribosomes on their stop codons and allows HaloTag maturation to continue in proximity to its UCI. Alternative approaches to promote controlled ribosomal stalling could include stop codon removal/suppression or use of a dominant negative release factor. Ribosome release could then be induced via addition of the chain terminator puromycin.

Since UCI cDNAs are formed on the 5′ UTR of the IVT-RNA, eukaryotic ribosomes would be unable to scan from the 5′ cap to the initiating Kozak sequence. The MIPSA system described here is therefore incompatible with cap-dependent eukaryotic cell-free translation systems. If cap-dependent translation is desired, however, two alternative methods could be developed. First, the current 5′ UCI system could be used if an internal ribosome entry site (IRES) were to be placed between the RT primer and the Kozak sequence. Second, the UCI could instead be introduced at the 3′ end of the RNA, provided that the RT was prevented from extending into the ORF. In an extension of eukaryotic MIPSA, RNA-cDNA hybrids could potentially be transfected into living cells or tissues, where UCI-protein formation could take place in situ, enabling many additional applications.

The ORF-associated UCIs can be embodied in a variety of ways. Here, stochastically assigned indexes were assigned to the human ORFeome at ˜10× representation. This approach has two main benefits: first, a single degenerate oligonucleotide pool is low cost; second, multiple independent measurements are reported by the ensemble of UCIs associated with each ORF. The library here was designed to have UCIs with uniform GC-content, and thus uniform PCR amplification efficiency. For simplicity, it was opted not to incorporate unique molecular identifiers (UMIs) into the RT primer, but this approach is compatible with MIPSA UCIs, and may potentially enhance quantitation. One disadvantage of stochastic indexing is the potential for ORF dropout, and thus the need for relatively high UCI representation; this increases the depth of sequencing required to quantify each UCI, and thus the overall per-sample cost. A second disadvantage is the requirement to construct a UCI-ORFeome matching dictionary. With short-read sequencing, the inventors were unable to disambiguate a fraction of the library, comprised mostly of alternative isoforms. Using a long-read sequencing technology, such as PacBio or Oxford Nanopore Technologies, instead of or in addition to short-read sequencing technology could surmount incomplete disambiguation. As opposed to stochastic barcoding, individual UCI-ORF cloning is possible but costly and cumbersome. However, a smaller UCI set would provide the advantage of lower per-assay sequencing cost. A methodology to clone ORFeomes using Long Adapter Single Stranded Oligonucleotide (LASSO) probes was previously developed.⁵⁰ LASSO cloning of ORFeome libraries thus naturally synergizes with MIPSA-based applications.

MIPSA Readout Via qPCR

A useful feature of appropriately designed UCIs is that they can also serve as qPCR readout probes. The degenerate UCIs that were designed and used here (FIG. 1B) comprise 18 nt base-balanced forward and reverse primer binding sites. The low cost and rapid turnaround time of a qPCR assay can thus be leveraged in combination with MIPSA. For example, incorporating assay quality control measures, such as the TRIM21 IP, can be used to qualify a set of samples prior to a more costly sequencing run. Troubleshooting and optimization can similarly be expedited by employing qPCR as a readout, rather than relying exclusively on NGS. qPCR testing of specific UCIs may theoretically also provide enhanced sensitivity compared to sequencing, and may be more amenable to analysis in a clinical setting.

### Conclusions

MIPSA is a self-assembling protein display technology with key advantages over alternative approaches. It has properties that complement techniques like PhIP-Seq, and MIPSA ORFeome libraries can be conveniently screened in the same reactions with phage display libraries. The MIPSA protocol presented here requires cap-independent, cell-free translation, but future adaptations may overcome this limitation. Applications for MIPSA-based studies include protein-protein, protein-antibody, and protein-small molecule interaction studies, as well as analyses of post-translational modifications. Here MIPSA was used to detect known autoantibodies and to discover neutralizing IFN-λ3 autoantibodies, among many other potentially pathogenic autoreactivities (Table 4) that may contribute to life-threatening COVID-19 in a subset of at-risk individuals.

TABLE 4
Proteins reactive in severe COVID-19 patients (continued on next page). Symbol, gene symbol; AAgAtlas, is
protein listed in AAgAtlas 1.0; #Severe, number of severe COVID-19 patients with reactivity to at least one UCI;
#Controls, number of control donors (healthy or mild-moderate COVID-19) with reactivity to at least one UCI;
#Reactive_UCIs, number of reactive UCIs associated with given ORF; Hits_FCs, mean and range (minimum to
maximum) of per-ORF maximum hits fold-change observed among the patients with the reactivity; Cluster_ID,
antigen cluster defined by FIG. 4B.

| Symbol | Gene_name | AAgAtlas | #Severe | #Controls | #Reactive_UCIs | hits_FCs | Cluster_ID |
|---|---|---|---|---|---|---|---|
| ASTL | astacin like | no | 2 | 1 | 2 | 5.7, | 1 |
| | metalloendopeptidase | | | | | (3.8, 7.5) | |
| BEND7 | BEN domain | no | 6 | 1 | 7 | 5.5, | 2 |
| | containing 7 | | | | | (3.2, 16.1) | |
| BLVRA | biliverdin | no | 1 | 0 | 3 | 17.9, | 1 |
| | reductase A | | | | | (17.9, 17.9) | |
| BMPR2 | bone morphogenetic | yes | 3 | 0 | 2 | 3.5, | 1 |
| | protein receptor type 2 | | | | | (3.2, 4.0) | |
| C1orf94 | chromosome 1 open | no | 12 | 0 | 8 | 5.2, | 3 |
| | reading frame 94 | | | | | (3.0, 15.4) | |
| C3orf18 | chromosome 3 open | no | 1 | 0 | 2 | 3.3, | 1 |
| | reading frame 18 | | | | | (3.3, 3.3) | |
| CALHM1 | calcium homeostasis | no | 3 | 1 | 2 | 3.9, | 1 |
| | modulator 1 | | | | | (3.3, 4.4) | |
| CAV2 | caveolin 2 | no | 9 | 0 | 2 | 3.7, | 4 |
| | | | | | | (3.1, 5.0) | |
| CCDC106 | coiled-coil domain | no | 4 | 0 | 10 | 4.4, | 2 |
| | containing 106 | | | | | (3.1, 7.2) | |
| CCDC146 | coiled-coil domain | no | 5 | 0 | 3 | 3.6, | 1 |
| | containing 146 | | | | | (3.1, 4.7) | |
| CD2BP2 | CD2 cytoplasmic tail | no | 2 | 0 | 3 | 14.9, | 5 |
| | binding protein 2 | | | | | (5.1, 24.8) | |
| CDC73 | cell division cycle 73 | no | 1 | 0 | 2 | 4.1, | 1 |
| | | | | | | (4.1, 4.1) | |
| CHMP7 | charged multivesicular | no | 10 | 1 | 3 | 3.6, | 4 |
| | body protein 7 | | | | | (3.1, 4.7) | |
| CTAG2 | cancer/testis | no | 3 | 0 | 6 | 5.2, | 1 |
| | antigen 2 | | | | | (3.0, 9.4) | |
| CYP2S1 | cytochrome P450 family 2 | no | 2 | 0 | 3 | 4.1, | 1 |
| | subfamily S member 1 | | | | | (3.2, 5.0) | |
| DNAJC17 | DnaJ heat shock protein | no | 4 | 0 | 2 | 3.2, | 2 |
| | family (Hsp40) member C17 | | | | | (3.0, 3.6) | |
| DOLPP1 | dolichyldiphosphatase 1 | no | 3 | 0 | 3 | 4.7, | 1 |
| | | | | | | (3.9, 5.1) | |
| EHD1 | EH domain containing 1 | no | 2 | 0 | 14 | 33.4, | 1 |
| | | | | | | (3.6, 63.2) | |
| EHD2 | EH domain containing 2 | no | 2 | 0 | 4 | 4.3, | 1 |
| | | | | | | (3.0, 5.6) | |
| ELOA2 | elongin A2 | no | 9 | 0 | 2 | 3.5, | 4 |
| | | | | | | (3.0, 4.9) | |
| EXD1 | exonuclease 3′-5′ | no | 1 | 0 | 2 | 7.2, | 5 |
| | domain containing 1 | | | | | (7.2, 7.2) | |
| EXOC4 | exocyst complex | no | 17 | 0 | 7 | 3.9, | 4 |
| | component 4 | | | | | (3.0, 4.9) | |
| FAM185A | family with sequence | no | 4 | 1 | 2 | 3.4, | 1 |
| | similarity 185 member A | | | | | (3.2, 3.6) | |
| FAM32A | family with sequence | no | 4 | 0 | 2 | 3.6, | 2 |
| | similarity 32 member A | | | | | (3.2, 4.0) | |
| FBXL19 | F-box and leucine rich | no | 2 | 0 | 3 | 7.0, | 1 |
| | repeat protein 19 | | | | | (3.0, 11.0) | |
| FDFT1 | farnesyl-diphosphate | no | 1 | 0 | 2 | 46.8, | 1 |
| | farnesyltransferase 1 | | | | | (46.8, 46.8) | |
| FRG1 | FSHD region gene 1 | no | 5 | 0 | 3 | 3.6, | 1 |
| | | | | | | (3.2, 4.3) | |
| FUT9 | fucosyltransferase 9 | no | 2 | 1 | 3 | 3.9, | 1 |
| | | | | | | (3.5, 4.3) | |
| GATA2 | GATA binding protein 2 | no | 5 | 0 | 2 | 3.6, | 4 |
| | | | | | | (3.0, 4.3) | |
| GIMAP8 | GTPase, IMAP family | no | 1 | 0 | 2 | 4.7, | 1 |
| | member 8 | | | | | (4.7, 4.7) | |
| HNF4A | hepatocyte nuclear | no | 1 | 0 | 2 | 11.7, | 1 |
| | factor 4 alpha | | | | | (11.7, 11.7) | |
| HNRNPUL1 | heterogeneous nuclear | no | 3 | 0 | 4 | 5.7, | 1 |
| | ribonucleoprotein U like 1 | | | | | (3.6, 8.6) | |
| HPGD | 15-hydroxyprostaglandin | no | 1 | 0 | 4 | 6.0, | 1 |
| | dehydrogenase | | | | | (6.0, 6.0) | |
| IFNA10 | interferon alpha 10 | no | 2 | 0 | 5 | 18.8, | 2 |
| | | | | | | (16.8, 20.7) | |
| IFNA13 | interferon alpha 13 | no | 4 | 0 | 2 | 22.5, | 2 |
| | | | | | | (4.6, 51.4) | |
| IFNA14 | interferon alpha 14 | no | 3 | 0 | 2 | 19.3, | 2 |
| | | | | | | (3.2, 44.2) | |
| IFNA2 | interferon alpha 2 | yes | 2 | 0 | 3 | 42.5, | 2 |
| | | | | | | (25.2, 59.8) | |
| IFNA21 | interferon alpha 21 | no | 2 | 0 | 10 | 25.1, | 2 |
| | | | | | | (14.9, 35.3) | |
| IFNA5 | interferon alpha 5 | no | 2 | 0 | 3 | 14.6, | 2 |
| | | | | | | (14.6, 14.7) | |
| IFNA6 | interferon alpha 6 | no | 4 | 1 | 12 | 9.4, | 2 |
| | | | | | | (3.3, 21.8) | |
| IFNA8 | interferon alpha 8 | no | 7 | 0 | 5 | 9.7, | 2 |
| | | | | | | (3.1, 36.4) | |
| IFNL3 | interferon lambda 3 | no | 3 | 1 | 5 | 5.5, | 1 |
| | | | | | | (4.2, 7.6) | |
| IFNW1 | interferon omega 1 | no | 2 | 0 | 5 | 29.6, | 2 |
| | | | | | | (10.6, 48.5) | |
| IKZF3 | IKAROS family zinc | no | 2 | 0 | 4 | 13.8, | 1 |
| | finger 3 | | | | | (3.3, 24.2) | |
| KCNJ12 | potassium inwardly rectifying | no | 1 | 0 | 2 | 3.1, | 1 |
| | channel subfamily J member 12 | | | | | (3.1, 3.1) | |
| KCNJ14 | potassium inwardly rectifying | no | 2 | 0 | 3 | 3.9, | 1 |
| | channel subfamily J member 14 | | | | | (3.2, 4.6) | |
| KLHL31 | kelch like family member 31 | no | 1 | 0 | 2 | 11.3, | 2 |
| | | | | | | (11.3, 11.3) | |
| KLHL40 | kelch like family member 40 | no | 1 | 0 | 4 | 8.4, | 2 |
| | | | | | | (8.4, 8.4) | |
| LALBA | lactalbumin alpha | no | 1 | 0 | 2 | 3.9, | 1 |
| | | | | | | (3.9, 3.9) | |
| LINC01547 | long intergenic non-protein | no | 2 | 0 | 6 | 19.3, | 1 |
| | coding RNA 1547 | | | | | (3.4, 35.1) | |
| MAGEE1 | MAGE family member E1 | no | 1 | 0 | 3 | 17.9, | 1 |
| | | | | | | (17.9, 17.9) | |
| MAX | MYC associated factor X | no | 7 | 0 | 10 | 13.0, | 3 |
| | | | | | | (3.1, 30.9) | |
| MBD3L1 | methyl-CpG binding domain | no | 2 | 0 | 5 | 8.1, | 1 |
| | protein 3 like 1 | | | | | (4.1, 12.2) | |
| MKX | mohawk homeobox | no | 6 | 1 | 3 | 3.8, | 4 |
| | | | | | | (3.1, 4.8) | |
| MPPED2 | metallophosphoesterase | no | 5 | 0 | 3 | 5.2, | 1 |
| | domain containing 2 | | | | | (3.1, 11.7) | |
| NACC1 | nucleus accumbens | no | 2 | 0 | 12 | 74.9, | 5 |
| | associated 1 | | | | | (74.7, 75.2) | |
| NAPSA | napsin A aspartic | no | 3 | 1 | 3 | 4.1, | 1 |
| | peptidase | | | | | (3.1, 4.7) | |
| NBPF1 | NBPF member 1 | no | 1 | 0 | 2 | 6.9, | 1 |
| | | | | | | (6.9, 6.9) | |
| NBPF15 | NBPF member 15 | no | 1 | 0 | 2 | 3.5, | 1 |
| | | | | | | (3.5, 3.5) | |
| NOXO1 | NADPH oxidase | no | 3 | 0 | 6 | 3.9, | 1 |
| | organizer 1 | | | | | (3.0, 4.8) | |
| NT5C1A | 5′-nucleotidase, | no | 3 | 1 | 2 | 26.9, | 5 |
| | cytosolic IA | | | | | (7.2, 59.9) | |
| NUP62 | nucleoporin 62 | no | 1 | 0 | 7 | 8.4, | 1 |
| | | | | | | (8.4, 8.4) | |
| NVL | nuclear VCP like | no | 1 | 0 | 2 | 21.6, | 1 |
| | | | | | | (21.6, 21.6) | |
| OLFM4 | olfactomedin 4 | yes | 5 | 1 | 3 | 12.9, | 5 |
| | | | | | | (4.4, 29.8) | |
| PIMREG | PICALM interacting | no | 4 | 1 | 4 | 3.8, | 1 |
| | mitotic regulator | | | | | (3.5, 4.1) | |
| PLEKHF1 | pleckstrin homology and | no | 2 | 0 | 3 | 3.3, | 1 |
| | FYVE domain containing 1 | | | | | (3.1, 3.5) | |
| PML | PML nuclear body | no | 1 | 0 | 4 | 29.7, | 1 |
| | scaffold | | | | | (29.7, 29.7) | |
| PNMA1 | PNMA family member 1 | yes | 1 | 0 | 3 | 6.4, | 2 |
| | | | | | | (6.4, 6.4) | |
| PNMA5 | PNMA family member 5 | no | 2 | 0 | 5 | 5.7, | 2 |
| | | | | | | (4.0, 7.4) | |
| POLDIP3 | DNA polymerase delta | no | 5 | 0 | 3 | 3.3, | 4 |
| | interacting protein 3 | | | | | (3.1, 3.7) | |
| POMP | proteasome maturation | no | 1 | 0 | 2 | 3.2, | 1 |
| | protein | | | | | (3.2, 3.2) | |
| POU6F1 | POU class 6 homeobox 1 | no | 1 | 0 | 3 | 12.0, | 1 |
| | | | | | | (12.0, 12.0) | |
| PQBP1 | polyglutamine binding | no | 5 | 0 | 2 | 3.2, | 5 |
| | protein 1 | | | | | (3.0, 3.5) | |
| PRKAR2B | protein kinase cAMP-dependent | no | 1 | 0 | 3 | 7.3, | 1 |
| | type II regulatory subunit beta | | | | | (7.3, 7.3) | |
| PXDNL | peroxidasin like | no | 4 | 0 | 4 | 3.5, | 2 |
| | | | | | | (3.1, 3.9) | |
| RBM17 | RNA binding motif protein 17 | no | 1 | 0 | 3 | 23.6, | 1 |
| | | | | | | (23.6, 23.6) | |
| RCAN3 | RCAN family member 3 | no | 1 | 0 | 5 | 5.3, | 1 |
| | | | | | | (5.3, 5.3) | |
| RPL13AP3 | ribosomal protein L13a | no | 4 | 1 | 5 | 3.4, | 1 |
| | pseudogene 3 | | | | | (3.1, 3.7) | |
| RPL15 | ribosomal protein L15 | no | 11 | 1 | 6 | 3.4, | 3 |
| | | | | | | (3.1, 3.9) | |
| RPP14 | ribonuclease P/MRP | no | 1 | 0 | 6 | 35.9, | 1 |
| | subunit p14 | | | | | (35.9, 35.9) | |
| RPP30 | ribonuclease P/MRP | no | 1 | 0 | 4 | 46.1, | 1 |
| | subunit p30 | | | | | (46.1, 46.1) | |
| RUFY4 | RUN and FYVE domain | no | 1 | 0 | 4 | 16.3, | 1 |
| | containing 4 | | | | | (16.3, 16.3) | |
| SNRPA1 | small nuclear ribonucleoprotein | no | 1 | 0 | 2 | 5.3, | 1 |
| | polypeptide A′ | | | | | (5.3, 5.3) | |
| SPEF1 | sperm flagellar 1 | no | 2 | 0 | 5 | 5.9, | 1 |
| | | | | | | (3.2, 8.5) | |
| SPRR1B | small proline rich | no | 1 | 0 | 4 | 7.5, | 1 |
| | protein 1B | | | | | (7.5, 7.5) | |
| SSNA1 | SS nuclear autoantigen 1 | yes | 1 | 0 | 5 | 12.2, | 2 |
| | | | | | | (12.2, 12.2) | |
| STPG3 | sperm-tail PG-rich | no | 1 | 0 | 2 | 3.6, | 1 |
| | repeat containing 3 | | | | | (3.6, 3.6) | |
| SYT2 | synaptotagmin 2 | no | 6 | 1 | 4 | 3.6, | 5 |
| | | | | | | (3.2, 4.5) | |
| TBC1D10B | TBC1 domain family | no | 3 | 1 | 2 | 3.4, | 1 |
| | member 10B | | | | | (3.1, 4.1) | |
| TFAP4 | transcription factor | no | 1 | 0 | 5 | 3.7, | 1 |
| | AP-4 | | | | | (3.7, 3.7) | |
| TMPO | thymopoietin | no | 2 | 0 | 5 | 15.7, | 1 |
| | | | | | | (3.7, 27.7) | |
| TNFSF14 | TNF superfamily | no | 1 | 0 | 2 | 3.7, | 1 |
| | member 14 | | | | | (3.7, 3.7) | |
| TOX4 | TOX high mobility group | no | 1 | 0 | 3 | 9.9, | 1 |
| | box family member 4 | | | | | (9.9, 9.9) | |
| TRAIP | TRAF interacting protein | no | 2 | 0 | 3 | 4.7, | 1 |
| | | | | | | (3.4, 6.0) | |
| VAV1 | vav guanine nucleotide | no | 1 | 0 | 4 | 10.2, | 1 |
| | exchange factor 1 | | | | | (10.2, 10.2) | |
| ZBTB18 | zinc finger and BTB | no | 1 | 0 | 2 | 3.3, | 1 |
| | domain containing 18 | | | | | (3.3, 3.3) | |
| ZFP2 | ZFP2 zinc finger protein | no | 2 | 1 | 3 | 4.9, | 1 |
| | | | | | | (3.1, 6.7) | |
| ZMAT2 | zinc finger matrin-type 2 | no | 6 | 0 | 5 | 3.6, | 2 |
| | | | | | | (3.1, 4.1) | |
| ZNF146 | zinc finger protein 146 | no | 2 | 0 | 8 | 16.4, | 1 |
| | | | | | | (3.1, 29.7) | |
| ZNF232 | zinc finger protein 232 | no | 2 | 1 | 4 | 4.6, | 1 |
| | | | | | | (3.5, 5.8) | |
| ZNF678 | zinc finger protein 678 | no | 3 | 1 | 2 | 6.6, | 1 |
| | | | | | | (3.4, 12.8) | |
| ZSCAN32 | zinc finger and SCAN | no | 1 | 0 | 2 | 11.3, | 1 |
| | domain containing 32 | | | | | (11.3, 11.3) | |
| ZSCAN5A | zinc finger and SCAN | no | 3 | 0 | 4 | 4.0, | 1 |
| | domain containing 5A | | | | | (3.2, 5.3) | |

### REFERENCES

1 Larman, H. B. et al. Cytosolic 5′-nucleotidase 1A autoimmunity in sporadic inclusion body myositis. Annals of neurology 73, 408-418, doi:10.1002/ana.23840 (2013).
2 Xu, G. J. et al. Viral immunology. Comprehensive serological profiling of human populations human virome. Science 348, aaa0698, doi:10.1126/science.aaa0698 (2015).
3 Shrock, E. et al. Viral epitope profiling of COVID-19 patients reveals cross-reactivity and correlates of severity. Science 370, doi:10.1126/science.abd4250 (2020).
4 Monaco, D. R. et al. Profiling serum antibodies with a pan allergen phage library identifies key wheat allergy epitopes. Nat Commun 12, 379, doi:10.1038/s41467-020-20622-1 (2021).
5 Kingsmore, S. F. Multiplexed protein measurement: technologies and applications of protein and antibody arrays. Nat Rev Drug Discov 5, 310-320, doi:10.1038/nrd2006 (2006).
6 Kodadek, T. Protein microarrays: prospects and problems. Chem Biol 8, 105-115, doi:10.1016/s1074-5521 (00) 90067-x (2001).
7 Ramachandran, N., Hainsworth, E., Demirkan, G. & LaBaer, J. On-chip protein synthesis for making microarrays. Methods Mol Biol 328, 1-14, doi:10.1385/1-59745-026-X: 1 (2006).
8 Rungpragayphan, S., Yamane, T. & Nakano, H. SIMPLEX: single-molecule PCR-linked in vitro expression: a novel method for high-throughput construction and screening of protein libraries. Methods Mol Biol 375, 79-94, doi:10.1007/978-1-59745-388-2_4 (2007).
9 Zhu, J. et al. Protein interaction discovery using parallel analysis of translated ORFs (PLATO). Nat Biotechnol 31, 331-334, doi:10.1038/nbt.2539 (2013).
10 Liszczak, G. & Muir, T. W. Nucleic Acid-Barcoding Technologies: Converting DNA Sequencing into a Broad-Spectrum Molecular Counter. Angew Chem Int Ed Engl 58, 4144-4162, doi:10.1002/anie.201808956 (2019).
11 Los, G. V. et al. HaloTag: a novel protein labeling technology for cell imaging and protein analysis. ACS Chem Biol 3, 373-382, doi:10.1021/cb800025k (2008).
12 Yazaki, J. et al. HaloTag-based conjugation of proteins to barcoding-oligonucleotides. Nucleic Acids Res 48, e8, doi:10.1093/nar/gkz1086 (2020).
13 Liu, Y., Sawalha, A. H. & Lu, Q. COVID-19 and autoimmune diseases. Curr Opin Rheumatol 33, 155-162, doi:10.1097 (2021).
14 Knight, J. S. et al. The intersection of COVID-19 and autoimmunity. J Clin Invest 131, doi:10.1172/JCI154886 (2021).
15 Wang, E. Y. et al. Diverse functional autoantibodies in patients with COVID-19. Nature 595, 283-288, doi:10.1038/s41586-021-03631-y (2021).
16 Bastard, P. et al. Autoantibodies neutralizing type I IFNs are present in ˜4% of uninfected individuals over 70 years old and account for ˜20% of COVID-19 deaths. Sci Immunol 6, doi:10.1126/sciimmunol.ab14340 (2021).
17 Abers, M. S. et al. Neutralizing type-I interferon autoantibodies are associated with delayed viral clearance and intensive care unit admission in patients with COVID-19. Immunol Cell Biol 99, 917-921, doi:10.1111/imcb. 12495 (2021).
18 Mohammad, F., Green, R. & Buskirk, A. R. A systematically-revised ribosome profiling method for bacteria reveals pauses at single-codon resolution. Elife 8, doi:10.7554/eLife.42591 (2019).
19 Gu, L. et al. Multiplex single-molecule interaction profiling of DNA-barcoded proteins. Nature 515, 554-557, doi:10.1038/nature13761 (2014).
20 Yang, X. et al. A public genome-scale lentiviral expression library of human ORFs. Nat Methods 8, 659-661, doi:10.1038/nmeth. 1638 (2011).
21 Consiglio, C. R. et al. The Immunology of Multisystem Inflammatory Syndrome in Children with COVID-19. Cell 183, 968-981 e967, doi:10.1016/j.cell.2020.09.016 (2020).
22 Bastard, P. et al. Autoantibodies against type I IFNs in patients with life-threatening COVID-19. Science 370, doi:10.1126/science.abd4585 (2020).
23 Zuo, Y. et al. Prothrombotic autoantibodies in serum from patients hospitalized with COVID-19. Sci Transl Med 12, doi:10.1126/scitranslmed.abd3876 (2020).
24 Casciola-Rosen, L. et al. IgM autoantibodies recognizing ACE2 are associated with severe COVID-19. medRxiv, doi:10.1101/2020.10.13.20211664 (2020).
25 Woodruff, M. C., Ramonell, R. P., Lee, F. E. & Sanz, I. Broadly-targeted autoreactivity is common in severe SARS-CoV-2 Infection. medRxiv, doi:10.1101/2020.10.21.20216192 (2020).
26 Wang, D. et al. AAgAtlas 1.0: a human autoantigen database. Nucleic Acids Res 45, D769-D776, doi:10.1093/nar/gkw946 (2017).
27 Lloyd, T. E. et al. Cytosolic 5′-Nucleotidase 1A As a Target of Circulating Autoantibodies in Autoimmune Diseases. Arthritis Care Res (Hoboken) 68, 66-71, doi:10.1002/acr.22600 (2016).
28 Gupta, S., Nakabo, S., Chu, J., Hasni, S. & Kaplan, M. J. Association between anti-interferon-alpha autoantibodies and COVID-19 in systemic lupus erythematosus. medRxiv, doi:10.1101/2020.10.29.20222000 (2020).
29 Xu, G. J. et al. Systematic autoantigen analysis identifies a distinct subtype of scleroderma with coincident cancer. Proc Natl Acad Sci USA, doi:10.1073/pnas.1615990113 (2016).
30 Venkataraman, T. et al. Analysis of antibody binding specificities in twin and SNP-genotyped cohorts reveals that antiviral antibody epitope selection is a heritable trait. Immunity 55, 174-184 e175, doi:10.1016/j.immuni.2021.12.004 (2022).
31 Stoeckius, M. et al. Simultaneous epitope and transcriptome measurement in single cells. Nat Methods 14, 865-868, doi:10.1038/nmeth.4380 (2017).
32 Setliff, I. et al. High-Throughput Mapping of B Cell Receptor Sequences to Antigen Specificity. Cell 179, 1636-1646 e1615, doi:10.1016/j.cell.2019.11.003 (2019).
33 Saka, S. K. et al. Immuno-SABER enables highly multiplexed and amplified protein imaging in tissues. Nat Biotechnol 37, 1080-1090, doi:10.1038/s41587-019-0207-y (2019).
34 Roman-Melendez, G. D. et al. Citrullination of a phage-displayed human peptidome library reveals the fine specificities of rheumatoid arthritis-associated autoantibodies. EBioMedicine 71, 103506, doi:10.1016/j.ebiom.2021.103506 (2021).
35 Roman-Melendez, G. D., Venkataraman, T., Monaco, D. R. & Larman, H. B. Protease Activity Profiling via Programmable Phage Display of Comprehensive Proteome-Scale Peptide Libraries. Cell Syst 11, 375-381 e374, doi:10.1016/j.cels.2020.08.013 (2020).
36 Mordstein, M. et al. Lambda interferon renders epithelial cells of the respiratory and 36 gastrointestinal tracts resistant to viral infections. J Virol 84, 5670-5677, doi:10.1128/JVI.00272-10 (2010).
37 Ank, N. et al. Lambda interferon (IFN-lambda), a type III IFN, is induced by viruses and IFNs and displays potent antiviral activity against select virus infections in vivo. J Virol 80, 4501-4509, doi:10.1128/JVI.80.9.4501-4509.2006 (2006).
38 Busnadiego, I. et al. Antiviral Activity of Type I, II, and III Interferons Counterbalances ACE2 Inducibility and Restricts SARS-CoV-2. mBio 11, doi:10.1128/mBio.01928-20 (2020).
39 Vanderheiden, A. et al. Type I and Type III Interferons Restrict SARS-CoV-2 Infection of Human Airway Epithelial Cultures. J Virol 94, doi:10.1128/JVI.00985-20 (2020).
40 Stanifer, M. L. et al. Critical Role of Type III Interferon in Controlling SARS-CoV-2 Infection in Human Intestinal Epithelial Cells. Cell Rep 32, 107863, doi:10.1016/j.celrep.2020.107863 (2020).
41 Galani, I. E. et al. Untuned antiviral immunity in COVID-19 revealed by temporal type I/III interferon patterns and flu comparison. Nat Immunol 22, 32-40, doi:10.1038/s41590-020-00840-x (2021).
42 Felgenhauer, U. et al. Inhibition of SARS-CoV-2 by type I and type III interferons. J Biol Chem 295, 13958-13964, doi:10.1074/jbc.AC120.013788 (2020).
43 O'Brien, T. R. et al. Weak Induction of Interferon Expression by Severe Acute Respiratory Syndrome Coronavirus 2 Supports Clinical Trials of Interferon-lambda to Treat Early Coronavirus Disease 2019. Clin Infect Dis 71, 1410-1412, doi:10.1093/cid/ciaa453 (2020).
44 Andreakos, E. & Tsiodras, S. COVID-19: lambda interferon against viral load and hyperinflammation. EMBO Mol Med 12, e12465, doi:10.15252/emmm.202012465 (2020).
45 Prokunina-Olsson, L. et al. COVID-19 and emerging viral infections: The case for interferon lambda. J Exp Med 217, doi:10.1084/jem.20200653 (2020).
46 Feld, J. J. et al. Peginterferon lambda for the treatment of outpatients with COVID-19: a phase 2, placebo-controlled randomised trial. Lancet Respir Med, doi:10.1016/S2213-2600 (20) 30566-X (2021).
47 Jongsma, M. A. & Litjens, R. H. Self-assembling protein arrays on DNA chips by auto-labeling fusion proteins with a single DNA address. Proteomics 6, 2650-2655, doi:10.1002/pmic.200500654 (2006).
48 Gautier, A. et al. An engineered protein tag for multiprotein labeling in living cells. Chem Biol 15, 128-136, doi:10.1016/j.chembiol.2008.01.007 (2008).
49 Samelson, A. J. et al. Kinetic and structural comparison of a protein's cotranslational folding and refolding pathways. Sci Adv 4, eaas9098, doi:10.1126/sciadv.aas9098 (2018).
50 Tosi, L. et al. Long-adapter single-strand oligonucleotide probes for the massively multiplexed cloning of kilobase genome regions. Nat Biomed Eng 1, doi:10.1038/s41551-017-0092 (2017).
51 Mohan, D. et al. Publisher Correction: PhIP-Seq characterization of serum antibodies using oligonucleotide-encoded peptidomes. Nature protocols 14, 2596, doi:10.1038/s41596-018-0088-4 (2019).
52 Tuckey, C., Asahara, H., Zhou, Y. & Chong, S. Protein synthesis using a reconstituted cell-free system. Curr Protoc Mol Biol 108, 16 31 11-22, doi:10.1002/0471142727.mb1631s108 (2014).
53 Klein, S. L. et al. Sex, age, and hospitalization drive antibody responses in a COVID-19 convalescent plasma donor population. J Clin Invest 130, 6141-6150, doi:10.1172/JCI142004 (2020).
54 Correction: Patient Trajectories Among Persons Hospitalized for COVID-19. Ann Intern Med 174, 144, doi:10.7326/L20-1322 (2021).
55 Zyskind I, R. A., Zimmerman J, Naiditch H, Glatt A E, Pinter A, Theel E S, Joyner M J, Hill D A, Lieberman M R, Bigajer E, Stok D, Frank E, Silverberg J I. SARS-CoV-2 Seroprevalence and Symptom Onset in Culturally-Linked Orthodox Jewish Communities Across Multiple Regions in the United States. JAMA Open Network In Press (2021).
56 Rose, M. R. & Group, E. I. W. 188th ENMC International Workshop: Inclusion Body Myositis, 2-4 Dec. 2011, Naarden, The Netherlands. Neuromuscul Disord 23, 1044-1055, doi:10.1016/j.nmd.2013.08.007 (2013).
57 Wei, Z., Zhang, W., Fang, H., Li, Y. & Wang, X. esATAC: an easy-to-use systematic pipeline for ATAC-seq data analysis. Bioinformatics 34, 2664-2665, doi:10.1093/bioinformatics/bty 141 (2018).
58 Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
59 brandonsie.github.io/epitopefindr/.

1. A method comprising the steps of:
(a) transcribing a vector library into messenger ribonucleic acid (mRNA), wherein the vector library encodes a plurality of proteins, and wherein each vector of the vector library comprises in the 5′ to 3′ direction:
(i) a polymerase transcriptional start site;
(ii) a barcode;
(iii) a reverse transcription primer binding site;
(iv) a ribosome binding site (RBS); and
(v) a nucleotide sequence encoding a fusion protein comprising (1) a polypeptide tag and (2) a protein, wherein the polypeptide tag specifically binds a ligand;
(b) reverse transcribing the 5′ end of the mRNA using a primer that binds upstream of the RBS, wherein the primer is conjugated with the ligand that specifically binds the polypeptide tag of the fusion protein, and wherein a complementary deoxyribonucleic acid (cDNA) is formed comprising the ligand, primer and barcode; and
(c) translating the mRNA, wherein the ligand of the cDNA binds the polypeptide tag of the fusion protein.

2. The method of claim 1, wherein the vector library is nicked prior to step (a).

3. The method of claim 1, wherein the vector further comprises (vi) an endonuclease site for vector linearization and the vector library is linearized prior to step (a).

4. The method of claim 1, wherein the barcode of the vector is flanked by binding sites for polymerase chain reaction (PCR) primers.

5. The method of claim 1, wherein the barcode comprises binding sites for PCR primers.

6. The method of claim 1, wherein the RBS comprises an internal ribosome entry site.

7. The method of claim 1, wherein the polypeptide tag is fused to the N-terminal end of the protein of interest.

8. The method of claim 1, wherein the polypeptide tag comprises haloalkane dehalogenase or 06-alkylguanine-DNA-alkyltransferase.

9. The method of claim 1, wherein the polypeptide tag comprises a HALO-tag and the ligand comprises a HALO-ligand.

10. The method of claim 9, wherein the HALO-tag comprises the amino acid sequence set forth in SEQ ID NO:22.

11. The method of claim 9, wherein the HALO-ligand comprises one of:

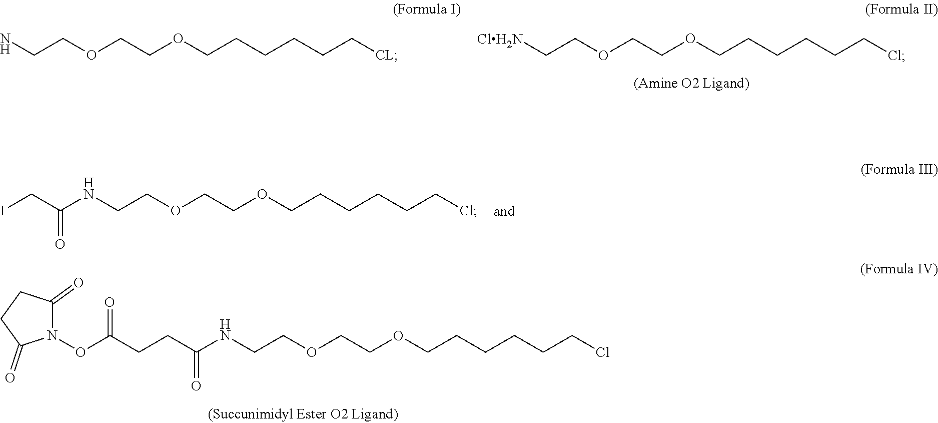

12. The method of claim 1, wherein the polypeptide tag comprises a SNAP-tag and the ligand comprises a SNAP-ligand.

13-17. (canceled)

18. A library of self-assembled protein-DNA conjugates wherein each protein-DNA conjugate comprises (a) a cDNA comprising a barcode, wherein the cDNA is conjugated with a ligand that specifically binds a polypeptide tag; and (b) a fusion protein comprising the polypeptide tag and a protein of interest, wherein the ligand is covalently bound to the polypeptide tag.

19-31. (canceled)

32. A method for studying protein-protein interactions comprising the step of performing a pull-down assay of the library of claim 1 with a protein of interest.

33. A method for studying protein-small molecule interactions comprising the step of performing a pull-down assay of the library of claim 1 with a small molecule.

34. A method comprising the step of performing an immunoprecipitation of the library of claim 1 with antibodies obtained from a biological sample.

35. A method for identifying the target of a first small molecule comprising the steps of (a) incubating the library of claim 1 with the first small molecule that binds its target(s) and (b) performing a pull-down assay of the library of step (a) with a second small molecule, wherein the first small molecule bound to its target(s) blocks the binding of the second small molecule.

36. A self-assembled protein-DNA composition comprising (a) a cDNA comprising a barcode, wherein the cDNA is conjugated with a ligand that specifically binds a polypeptide tag; and (b) a fusion protein comprising the polypeptide tag and a protein of interest, wherein the ligand is covalently bound to the polypeptide tag, or
A self-assembled protein display library comprising a plurality of vectors each comprising a nucleic acid sequence that encodes a protein of interest, wherein the plurality of vectors each comprise along the 5′ to 3′ direction:
(a) a polymerase transcriptional start site;
(b) a barcode;
(c) a reverse transcription primer binding site;
(d) a RBS; and
a nucleotide sequence encoding a fusion protein comprising (i) a polypeptide tag and (ii) a protein of interest, wherein the polypeptide tag specifically binds a ligand.

37-65. (canceled)

66. A vector comprising along the 5′ to 3′ direction:
(a) a polymerase transcriptional start site;
(b) a barcode;
(c) a reverse transcription primer binding site;
(d) a RBS; and
(e) a nucleotide sequence encoding a fusion protein comprising (i) a polypeptide tag and (ii) a protein of interest, wherein the polypeptide tag specifically binds a ligand,
or
A method comprising the steps of:
(a) transcribing a linearized or nicked plurality of vectors comprising the self-assembled protein display library of claim 50 to produce mRNA;
(b) reverse transcribing the 5′ end of the mRNA to produce cDNA comprising the barcodes using a primer conjugated to the ligand; and
(c) translating the mRNA,
wherein the polypeptide tag of the fusion protein covalently binds the ligand conjugated to the cDNA comprising the barcode.

67-82. (canceled)

83. A method for treating a patient having severe COVID-19 comprising the step of administering to the patient an effective amount of interferon therapy, wherein autoantibodies that neutralize IFN-λ3 are detected in a biological sample obtained from the patient,
or
A method for treating a patient having severe COVID-19 comprising the steps of:
(a) detecting autoantibodies that neutralize IFN-λ3 in a biological sample obtained from the patient; and
(b) treating the patient with an effective amount of interferon therapy,
or
A method for identifying a COVID-19 patient who would benefit from interferon therapy comprising the step of detecting autoantibodies that neutralize IFN-λ3 in a biological sample obtained from the patient.

84-87. (canceled)


SEQUENCE LISTING (24 sequences)

SEQ ID NO: 1 - DNA, 20 bp, Artificial Sequence
  T7-Pep2_PCR1_F
ataaaggtgagggtaatgtc

SEQ ID NO: 2 - DNA, 39 bp, Artificial Sequence
  Nextera Index 1 Read
caagcagaagacggcatacgagatgtctcgtgggctcgg

SEQ ID NO: 3 - DNA, 49 bp, Artificial Sequence
  PhIP_PCR2_F
aatgatacggcgaccaccgagatctacacggagctgtcgtattccagtc

SEQ ID NO: 4 - DNA, 21 bp, Artificial Sequence
  P7.2
caagcagaagacggcatacga

SEQ ID NO: 5 - DNA, 48 bp, Artificial Sequence
  T7-Pep2_PCR1_R+ad_min
ctggagttcagacgtgtgctcttccgatcagttactcgagcttatcgt

SEQ ID NO: 6 - DNA, 39 bp, Artificial Sequence
  Ad_min_BCX_P7
caagcagaagacggcatacgagatctggagttcagacgt

SEQ ID NO: 7 - DNA, 26 bp, Artificial Sequence
  T7-Pep2.2_SP_subA
ctcggggatccaggaattccgctgcg

SEQ ID NO: 8 - DNA, 50 bp, Artificial Sequence
  MISEQ_PLATO_R2
atgacgacaagccatggtcgaatcaaacaagtttgtacaaaaaagttggc

SEQ ID NO: 9 - DNA, 30 bp, Artificial Sequence
  Plato2_i5_NextSeq_SP
ggatccccgagactggaatacgacagctcc

SEQ ID NO: 10 - DNA, 33 bp, Artificial Sequence
  Standard_i7_SP
gatcggaagagcacacgtctgaactccagtcac

SEQ ID NO: 11 - DNA, 48 bp, Artificial Sequence
  HL-32_ad 5′ amine modified oligo--reverse transcription primer
gacgtgtgctcttccgatcaaattatttctaggtactcgagcttatcg

SEQ ID NO: 12 - DNA, 20 bp, Artificial Sequence
  MX1_Forward
accacagaggctctcagcat

SEQ ID NO: 13 - DNA, 20 bp, Artificial Sequence
  MX1_Reverse
ctcagctggtcctggatctc

SEQ ID NO: 14 - DNA, 20 bp, Artificial Sequence
  GAPDH_Forward
gagtcaacggatttggtcgt

SEQ ID NO: 15 - DNA, 20 bp, Artificial Sequence
  GAPDH_Reverse
ttgattttggagggatctcg

SEQ ID NO: 16 - DNA, 18 bp, Artificial Sequence
  BT2_F
gtcagagtgacacactgt

SEQ ID NO: 17 - DNA, 18 bp, Artificial Sequence
  BT2_R
agagtgacagtcacagtg

SEQ ID NO: 18 - DNA, 18 bp, Artificial Sequence
  BG4_F
cactgactgtgtgagtgt

SEQ ID NO: 19 - DNA, 18 bp, Artificial Sequence
  BG4_R
tgagacacagtgagtcac

SEQ ID NO: 20 - DNA, 17 bp, Artificial Sequence
  NT5C1A_F
ctcacagacagacgtca

SEQ ID NO: 21 - DNA, 18 bp, Artificial Sequence
  NT5C1A_R
tgtcagtcagtgagtgtg

SEQ ID NO: 22 - protein, 297 aa, Artificial Sequence
  HALO-Tag sequence
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 23 - protein, 182 aa, Artificial Sequence
  SNAP-Tag sequence
GPGSDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKR

SEQ ID NO: 24 - protein, 182 aa, Artificial Sequence
  CLIP-Tag sequence
MDKDCEMKRTTLDSPLGKLELSGCEQGLHRIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLG